(19) United States
(12) Patent Application Publication
(10) Pub. No.: US 2026/0239874 A1
(43) Pub. Date: Aug. 13, 2026

(54) COMPOUND FOR ORGANIC OPTOELECTRONIC DEVICE, ORGANIC OPTOELECTRONIC DEVICE AND DISPLAY DEVICE

(71) Applicant: SAMSUNG SDI CO., LTD., Yongin-si, Gyeonggi-do (KR)

(72) Inventors: Junghoon KIM, Suwon-si, Gyeonggi-do (KR); Youngmook LIM, Suwon-si, Gyeonggi-do (KR); Jongwoo WON, Suwon-si, Gyeonggi-do (KR); Sunwoong SHIN, Suwon-si, Gyeonggi-do (KR); Gibeom PARK, Suwon-si, Gyeonggi-do (KR); Kyoungrok KIM, Suwon-si, Gyeonggi-do (KR); Ho Kuk JUNG, Suwon-si, Gyeonggi-do (KR); Youngkyoung JO, Suwon-si, Gyeonggi-do (KR); Hyunji YOO, Suwon-si, Gyeonggi-do (KR); Min Seok SEO, Suwon-si, Gyeonggi-do (KR); Hyung Sun KIM, Suwon-si, Gyeonggi-do (KR)

(21) Appl. No.: 19/147,803

(22) PCT Filed: Jun. 12, 2024
(86) PCT No.: PCT/KR2024/008037

§ 371 Date: Jul. 14, 2025

(30) Foreign Application Priority Data
Jun. 14, 2023 (KR) 10-2023-0076193

(51) Int. Cl. H10K 85/60; C07B 59/00; C07D 307/91; C07D 333/76; C07D 407/12; C07D 409/04; C07D 409/12; C07F 7/08; H10K 50/11; H10K 50/15

(52) U.S. Cl. CPC H10K 85/633; C07B 59/002; C07D 307/91; C07D 333/76; C07D 407/12; C07D 409/04; C07D 409/12; C07F 7/0816; H10K 85/636; H10K 50/11; H10K 50/156; H10K 85/615; H10K 85/626; H10K 85/6574

(57) ABSTRACT

Disclosed are a compound for an organic optoelectronic device represented by Chemical Formula 1, an organic optoelectronic device including the same, and a display device.

The details of Chemical Formula 1 are as defined in the specification.

### TECHNICAL FIELD

A compound for an organic optoelectronic device, an organic optoelectronic device, and a display device are disclosed.

### BACKGROUND ART

An organic optoelectronic device (organic optoelectronic diode) is a device capable of converting electrical energy and optical energy to each other.

Organic optoelectronic devices may be largely divided into two types according to a principle of operation. One is a photoelectric device that generates electrical energy by separating excitons formed by light energy into electrons and holes, and transferring the electrons and holes to different electrodes, respectively and the other is light emitting device that generates light energy from electrical energy by supplying voltage or current to the electrodes.

Examples of the organic optoelectronic device include an organic photoelectric device, an organic light emitting diode, an organic solar cell, and an organic photoconductor drum.

Among them, organic light emitting diodes (OLEDs) are attracting much attention in recent years due to increasing demands for flat panel display devices. The organic light emitting diode is a device that converts electrical energy into light, and the performance of the organic light emitting diode is greatly influenced by an organic material between electrodes.

### DISCLOSURE

### Technical Problem

An embodiment provides a compound for an organic optoelectronic device capable of implementing a low-driving, high-efficiency, and long life-span organic optoelectronic device.

Another embodiment provides an organic optoelectronic device including the compound for the organic optoelectronic device.

Another embodiment provides a display device including the organic optoelectronic device.

### Technical Solution

According to one embodiment, a compound for an organic optoelectronic device represented by Chemical Formula 1 is provided.

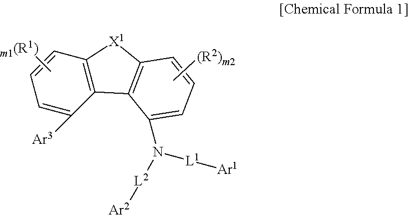

In Chemical Formula 1,
X¹ is O or S
R¹ and R² are each independently hydrogen, deuterium, a cyano group, a substituted or unsubstituted C1 to C20 alkyl group, a substituted or unsubstituted C6 to C20 aryl group, or a combination thereof,
Ar¹ and Ar² are each independently a substituted or unsubstituted phenyl group, a substituted or unsubstituted biphenyl group, a substituted or unsubstituted terphenyl group, a substituted or unsubstituted naphthyl group, a substituted or unsubstituted triphenylene group, a substituted or unsubstituted 1-fluorenyl group, a substituted or unsubstituted 2-fluorenyl group, a substituted or unsubstituted 3-fluorenyl group, a substituted or unsubstituted 4-fluorenyl group, a substituted or unsubstituted 9,9′-spirobifluorenyl group, a substituted or unsubstituted dibenzofuranyl group, a substituted or unsubstituted dibenzothiophenyl group, a substituted or unsubstituted dibenzosilolyl group, a substituted or unsubstituted spirocyclopentane-1,9′-fluorenyl group, a substituted or unsubstituted spirocyclohexane-1,9′-fluorenyl group, or a substituted or unsubstituted spirofluorene-9,9′-xanthenyl group,
Ar³ is a substituted or unsubstituted C6 to C20 aryl group or a substituted or unsubstituted C2 to C20 heterocyclic group,
L¹ and L² are each independently a single bond, a substituted or unsubstituted C6 to C20 arylene group, a substituted or unsubstituted C2 to C30 heterocyclic group, or a combination thereof,
m1 and m2 are each independently one of integers of 1 to 3, and
“substituted” refers to replacement of at least one hydrogen by deuterium, C1 to C5 alkyl group, a C6 to C12 aryl group, or a cyano group.

According to another embodiment, an organic optoelectronic device includes an anode and a cathode facing each other, and at least one organic layer between the anode and the cathode, wherein the organic layer includes the compound for the organic optoelectronic device.

According to another embodiment, a display device including the organic optoelectronic device is provided.

### Advantageous Effects

An organic optoelectronic device having high efficiency and a long life-span may be realized.

### DESCRIPTION OF THE DRAWINGS

FIG. 1 is a cross-sectional view illustrating an organic light emitting diode according to an embodiment.

### DESCRIPTION OF SYMBOLS

100: organic light emitting diode
105: organic layer
110: cathode
120: anode
130: light emitting layer
140: hole transport region
150: electron transport region

### BEST MODE

Hereinafter, embodiments of the present invention are described in detail. However, these embodiments are exemplary, and this disclosure is not limited thereto.

As used herein, when a definition is not otherwise provided, “substituted” refers to replacement of at least one hydrogen of a substituent or a compound by deuterium, a halogen, a hydroxyl group, an amino group, a substituted or unsubstituted C1 to C30 amine group, a nitro group, a substituted or unsubstituted C1 to C40 silyl group, a C1 to C30 alkyl group, a C1 to C10 alkylsilyl group, a C6 to C30 arylsilyl group, a C3 to C30 cycloalkyl group, a C3 to C30 heterocycloalkyl group, a C6 to C30 aryl group, a C2 to C30 heteroaryl group, a C1 to C20 alkoxy group, a C1 to C10 trifluoroalkyl group, a cyano group, or a combination thereof.

In one example of the present invention, the “substituted” refers to replacement of at least one hydrogen of a substituent or a compound by deuterium, a C1 to C30 alkyl group, a C1 to C10 alkylsilyl group, a C6 to C30 arylsilyl group, a C3 to C30 cycloalkyl group, a C3 to C30 heterocycloalkyl group, a C6 to C30 aryl group, a C2 to C30 heteroaryl group, or a cyano group. In specific example of the present invention, the “substituted” refers to replacement of at least one hydrogen of a substituent or a compound by deuterium, a C1 to C20 alkyl group, a C6 to C30 aryl group, or a cyano group. In specific example of the present invention, the “substituted” refers to replacement of at least one hydrogen of a substituent or a compound by deuterium, a C1 to C5 alkyl group, a C6 to C18 aryl group, or a cyano group. In specific example of the present invention, the “substituted” refers to replacement of at least one hydrogen of a substituent or a compound by deuterium, a cyano group, a methyl group, an ethyl group, a propyl group, a butyl group, a phenyl group, a biphenyl group, a terphenyl group, or a naphthyl group.

In the present specification, “unsubstituted” refers to non-replacement of a hydrogen atom by another substituent and remaining of the hydrogen atom.

In the present specification, “deuterium substituted (-D)” may include “tritium substitution (-T).”

In this specification, “t-Bu” or “tBu” means a tert-butyl group.

As used herein, when a definition is not otherwise provided, “hetero” refers to one including one to three heteroatoms selected from N, O, S, P, and Si, and remaining carbons in one functional group.

As used herein, “aryl group” refers to a group including at least one hydrocarbon aromatic moiety, and all elements of the hydrocarbon aromatic moiety have p-orbitals which form conjugation, for example a phenyl group, a naphthyl group, and the like, two or more hydrocarbon aromatic moieties may be linked by a sigma bond and may be, for example a biphenyl group, a terphenyl group, a quarterphenyl group, and the like, and two or more hydrocarbon aromatic moieties are fused directly or indirectly to provide a non-aromatic fused ring, for example a fluorenyl group.

The aryl group may include a monocyclic, polycyclic, or fused ring polycyclic (i.e., rings sharing adjacent pairs of carbon atoms) functional group.

As used herein, “heterocyclic group” is a generic concept of a heteroaryl group, and may include at least one heteroatom selected from N, O, S, P, and Si instead of carbon (C) in a cyclic compound such as aryl group, a cycloalkyl group, a fused ring thereof, or a combination thereof. When the heterocyclic group is a fused ring, the entire ring or each ring of the heterocyclic group may include one or more heteroatoms.

As an example, “heteroaryl group” may refer to aryl group including at least one heteroatom selected from N, O, S, P, and Si. Two or more heteroaryl groups are linked by a sigma bond directly, or when the heteroaryl group includes two or more rings, the two or more rings may be fused. When the heteroaryl group is a fused ring, each ring may include one to three heteroatoms.

More specifically, the substituted or unsubstituted C6 to C30 aryl group may be a substituted or unsubstituted phenyl group, a substituted or unsubstituted naphthyl group, a substituted or unsubstituted anthracenyl group, a substituted or unsubstituted phenanthrenyl group, a substituted or unsubstituted naphthacenyl group, a substituted or unsubstituted pyrenyl group, a substituted or unsubstituted biphenyl group, a substituted or unsubstituted p-terphenyl group, a substituted or unsubstituted m-terphenyl group, a substituted or unsubstituted o-terphenyl group, a substituted or unsubstituted chrysenyl group, a substituted or unsubstituted benzophenanthrenyl group, a substituted or unsubstituted triphenylene group, a substituted or unsubstituted perylenyl group, a substituted or unsubstituted fluorenyl group, a substituted or unsubstituted indenyl group, or a combination thereof, but is not limited thereto.

More specifically, the substituted or unsubstituted C2 to C30 heterocyclic group may be a substituted or unsubstituted furanyl group, a substituted or unsubstituted thiophenyl group, a substituted or unsubstituted pyrrolyl group, a substituted or unsubstituted pyrazolyl group, a substituted or unsubstituted imidazolyl group, a substituted or unsubstituted triazolyl group, a substituted or unsubstituted oxazolyl group, a substituted or unsubstituted thiazolyl group, a substituted or unsubstituted oxadiazolyl group, a substituted or unsubstituted thiadiazolyl group, a substituted or unsubstituted pyridyl group, a substituted or unsubstituted pyrimidinyl group, a substituted or unsubstituted pyrazinyl group, a substituted or unsubstituted triazinyl group, a substituted or unsubstituted benzofuranyl group, a substituted or unsubstituted benzothiophenyl group, a substituted or unsubstituted benzimidazolyl group, a substituted or unsubstituted indolyl group, a substituted or unsubstituted quinolinyl group, a substituted or unsubstituted isoquinolinyl group, a substituted or unsubstituted quinazolinyl group, a substituted or unsubstituted quinoxalinyl group, a substituted or unsubstituted naphthyridinyl group, a substituted or unsubstituted benzoxazinyl group, a substituted or unsubstituted benzthiazinyl group, a substituted or unsubstituted acridinyl group, a substituted or unsubstituted phenazinyl group, a substituted or unsubstituted phenothiazinyl group, a substituted or unsubstituted phenoxazinyl group, a substituted or unsubstituted carbazolyl group, a substituted or unsubstituted dibenzofuranyl group, or a substituted or unsubstituted dibenzothiophenyl group, a substituted or unsubstituted benzonaphthofuranyl group, a substituted or unsubstituted benzonaphthothiophenyl group, a substituted or unsubstituted benzofuranofluorenyl group, a substituted or unsubstituted benzothiophenefluorenyl group, or a combination thereof, but are not limited thereto.

As used herein, hole characteristics refer to an ability to donate an electron to form a hole when an electric field is applied and that a hole formed in the anode may be easily injected into the light emitting layer and transported in the light emitting layer due to conductive characteristics according to a highest occupied molecular orbital (HOMO) level.

In addition, electronic characteristics refer to an ability to accept an electron when an electric field is applied and that electron formed in the cathode may be easily injected into the light emitting layer and transported in the light emitting layer due to conductive characteristics according to a lowest unoccupied molecular orbital (LUMO) level.

Hereinafter, a compound for an organic optoelectronic device according to an embodiment is described.

The compound for the organic optoelectronic device according to an embodiment is represented by Chemical Formula 1.

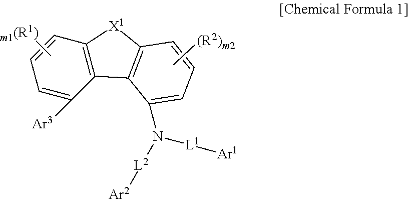

In Chemical Formula 1,
X¹ is O or S
R¹ and R² are each independently hydrogen, deuterium, a cyano group, a substituted or unsubstituted C1 to C20 alkyl group, a substituted or unsubstituted C6 to C20 aryl group, or a combination thereof,
Ar¹ and Ar² are each independently a substituted or unsubstituted phenyl group, a substituted or unsubstituted biphenyl group, a substituted or unsubstituted terphenyl group, a substituted or unsubstituted naphthyl group, a substituted or unsubstituted triphenylene group, a substituted or unsubstituted 1-fluorenyl group, a substituted or unsubstituted 2-fluorenyl group, a substituted or unsubstituted 3-fluorenyl group, a substituted or unsubstituted 4-fluorenyl group, a substituted or unsubstituted 9,9′-spirobifluorenyl group, a substituted or unsubstituted dibenzofuranyl group, a substituted or unsubstituted dibenzothiophenyl group, a substituted or unsubstituted dibenzosilolyl group, a substituted or unsubstituted spirocyclopentane-1,9′-fluorenyl group, a substituted or unsubstituted spirocyclohexane-1,9′-fluorenyl group, or a substituted or unsubstituted spirofluorene-9,9′-xanthenyl group,
Ar³ is a substituted or unsubstituted C6 to C20 aryl group or a substituted or unsubstituted C2 to C20 heterocyclic group,
L¹ and L² are each independently a single bond, a substituted or unsubstituted C6 to C20 arylene group, a substituted or unsubstituted C2 to C30 heterocyclic group, or a combination thereof,
m1 and m2 are each independently one of integers of 1 to 3, and
“substituted” refers to replacement of at least one hydrogen by deuterium, C1 to C5 alkyl group, a C6 to C12 aryl group, or a cyano group.

The compound represented by Chemical Formula 1 has a structure in which an amine group is substituted at the 1st position of dibenzofuran (or dibenzothiophene) and an aryl group is substituted at the 9th position. Due to stabilization through interaction between the 1st position substituent (amine group) and the 9th position substituent (aryl group), the driving voltage of an organic light emitting diode to which it is applied can be lowered and the life-span may be improved.

For example, L¹ and L² may each independently be a single bond, a substituted or unsubstituted phenylene group, or a substituted or unsubstituted biphenylene group.

For example, Ar¹ and Ar² may each independently be a substituted or unsubstituted phenyl group, a substituted or unsubstituted biphenyl group, a substituted or unsubstituted terphenyl group, a substituted or unsubstituted 1-fluorenyl group, a substituted or unsubstituted 2-fluorenyl group, a substituted or unsubstituted 3-fluorenyl group, a substituted or unsubstituted 4-fluorenyl group, a substituted or unsubstituted dibenzofuranyl group, a substituted or unsubstituted dibenzothiophenyl group, or a substituted or unsubstituted dibenzosilolyl group.

As a specific example, L¹-Ar¹ and L²-Ar² may each be independently selected from the substituents listed in Group I.

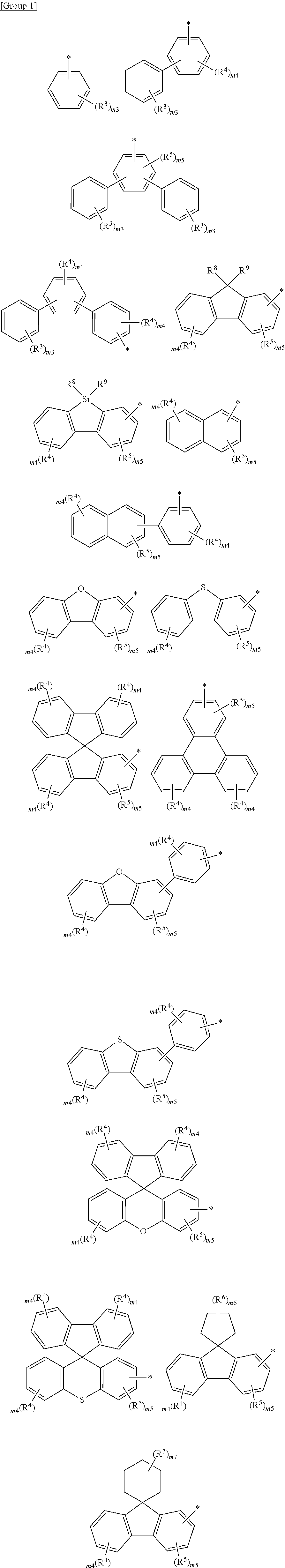

In Group I,
R³ to R⁷ are each independently hydrogen, deuterium, a cyano group, a substituted or unsubstituted C1 to C10 alkyl group, a substituted or unsubstituted C3 to C10 cycloalkyl group, or a substituted or unsubstituted C6 to C12 aryl group,
R¹ and R⁹ are each independently a substituted or unsubstituted C1 to C10 alkyl group or a substituted or unsubstituted C6 to C12 aryl group,
m3 is one of integers of 1 to 5,
m4 is one of integers of 1 to 4,
m5 is one of integers of 1 to 3,
m6 is one of integers of 1 to 8,
m7 is one of integers of 1 to 10, and
* is a linking point.

When m3 is 2 or more, each R³ may be the same or different from each other.

When m4 is 2 or more, each R⁴ may be the same or different from each other.

When m5 is 2 or more, each R⁵ may be the same or different from each other.

When m6 is 2 or more, each R⁶ may be the same or different from each other.

When m7 is 2 or more, each R⁷ may be the same or different from each other.

For example, Ar³ may be a substituted or unsubstituted phenyl group, a substituted or unsubstituted biphenyl group, a substituted or unsubstituted naphthyl group, a substituted or unsubstituted dibenzofuranyl group, or a substituted or unsubstituted dibenzothiophenyl group.

For example, the compound for the organic optoelectronic device represented by Chemical Formula 1 may be one selected from the compounds listed in Group 1, but is not limited thereto.

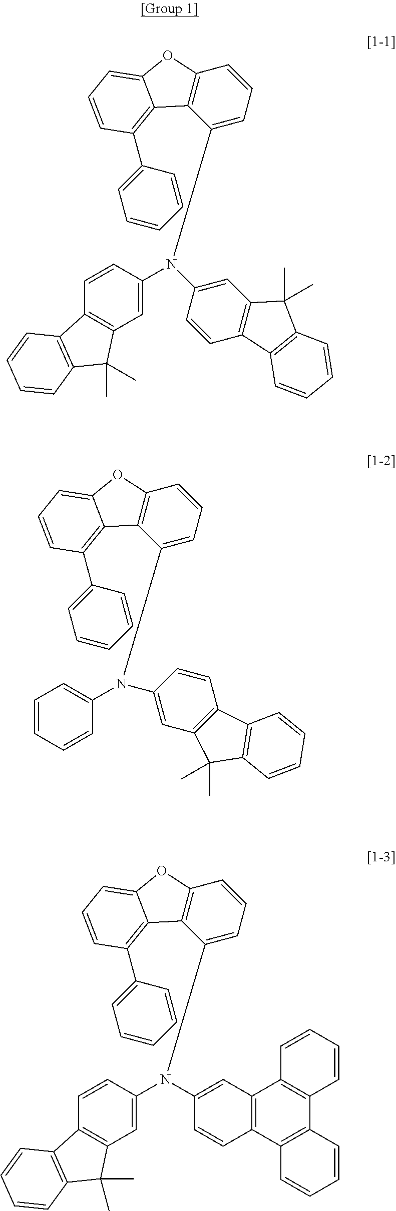 [SMILES: CC1(C)c2ccccc2-c2ccc(N(c3ccc4c(c3)C(C)(C)c3ccccc3-4)c3cccc4oc5cccc(-c6ccccc6)c5c34)cc21 ; CC1(C)c2ccccc2-c2ccc(N(c3ccccc3)c3cccc4oc5cccc(-c6ccccc6)c5c34)cc21 ; CC1(C)c2ccccc2-c2ccc(N(c3ccc4c5ccccc5c5ccccc5c4c3)c3cccc4oc5cccc(-c6ccccc6)c5c34)cc21]

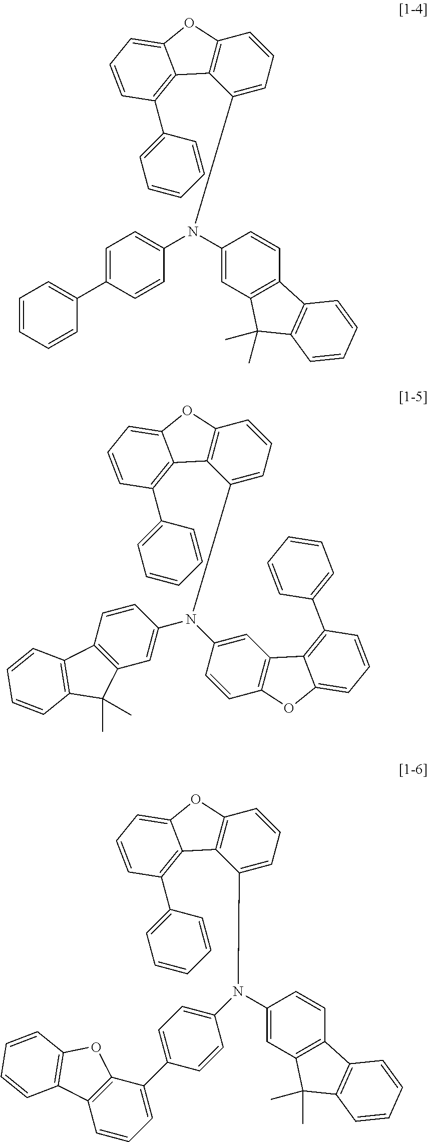 [SMILES: CC1(C)c2ccccc2-c2ccc(N(c3ccc(-c4ccccc4)cc3)c3cccc4oc5cccc(-c6ccccc6)c5c34)cc21 ; CC1(C)c2ccccc2-c2ccc(N(c3ccc4oc5cccc(-c6ccccc6)c5c4c3)c3cccc4oc5cccc(-c6ccccc6)c5c34)cc21 ; CC1(C)c2ccccc2-c2ccc(N(c3ccc(-c4cccc5c4oc4ccccc45)cc3)c3cccc4oc5cccc(-c6ccccc6)c5c34)cc21]

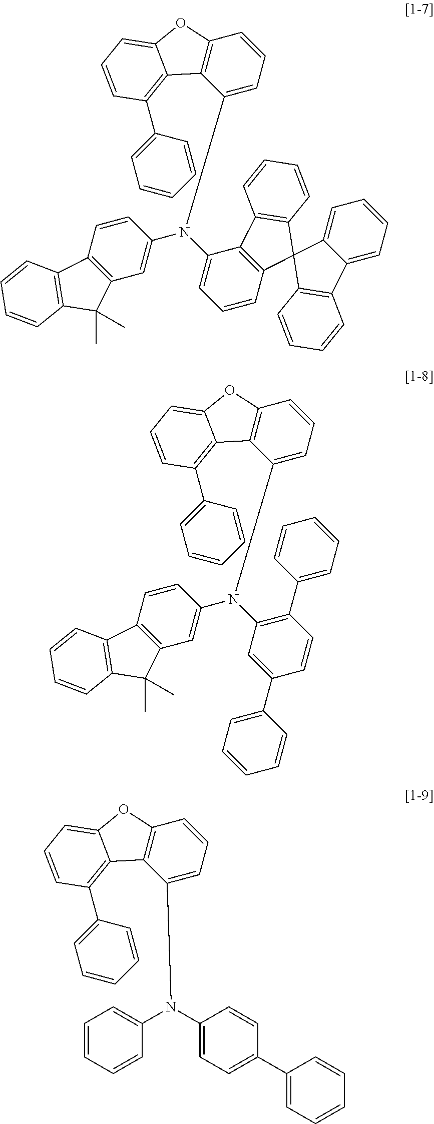 [SMILES: CC1(C)c2ccccc2-c2ccc(N(c3cccc4c3-c3ccccc3C43c4ccccc4-c4ccccc43)c3cccc4oc5cccc(-c6ccccc6)c5c34)cc21 ; CC1(C)c2ccccc2-c2ccc(N(c3cc(-c4ccccc4)ccc3-c3ccccc3)c3cccc4oc5cccc(-c6ccccc6)c5c34)cc21 ; c1ccc(-c2ccc(N(c3ccccc3)c3cccc4oc5cccc(-c6ccccc6)c5c34)cc2)cc1]

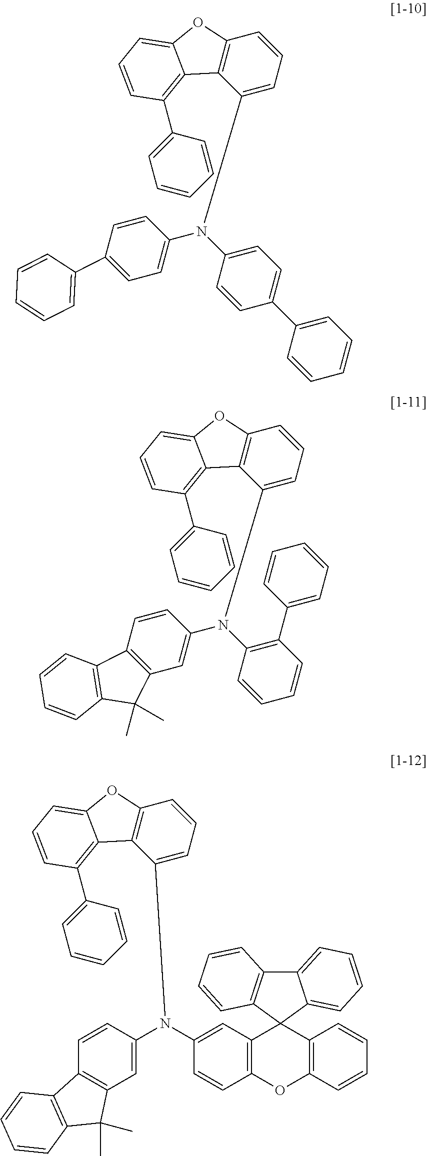 [SMILES: c1ccc(-c2ccc(N(c3ccc(-c4ccccc4)cc3)c3cccc4oc5cccc(-c6ccccc6)c5c34)cc2)cc1 ; CC1(C)c2ccccc2-c2ccc(N(c3ccccc3-c3ccccc3)c3cccc4oc5cccc(-c6ccccc6)c5c34)cc21 ; CC1(C)c2ccccc2-c2ccc(N(c3ccc4c(c3)C3(c5ccccc5O4)c4ccccc4-c4ccccc43)c3cccc4oc5cccc(-c6ccccc6)c5c34)cc21]

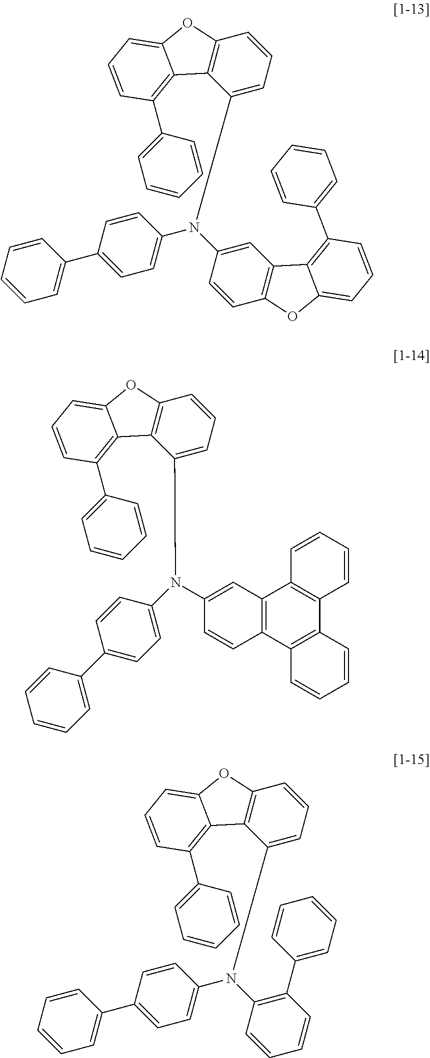 [SMILES: c1ccc(-c2ccc(N(c3ccc4oc5cccc(-c6ccccc6)c5c4c3)c3cccc4oc5cccc(-c6ccccc6)c5c34)cc2)cc1 ; c1ccc(-c2ccc(N(c3ccc4c5ccccc5c5ccccc5c4c3)c3cccc4oc5cccc(-c6ccccc6)c5c34)cc2)cc1 ; c1ccc(-c2ccc(N(c3ccccc3-c3ccccc3)c3cccc4oc5cccc(-c6ccccc6)c5c34)cc2)cc1]

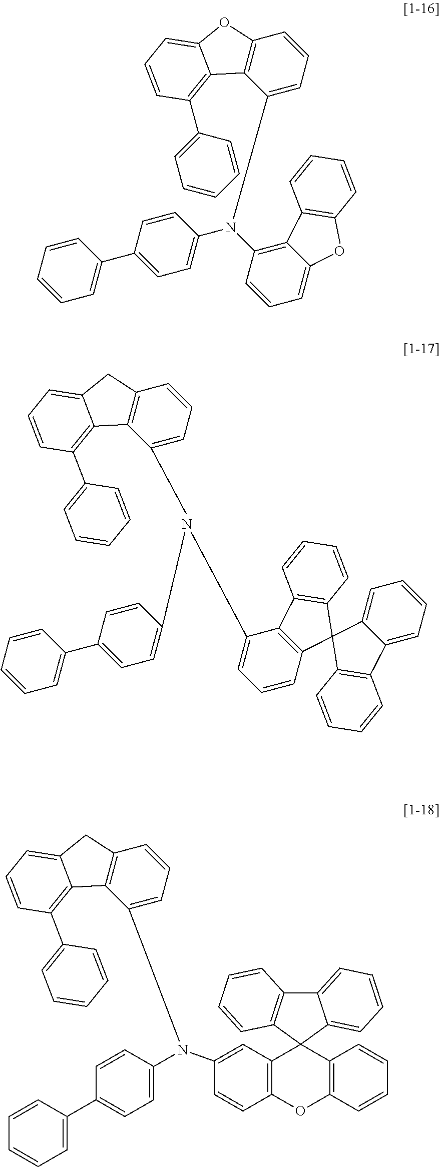

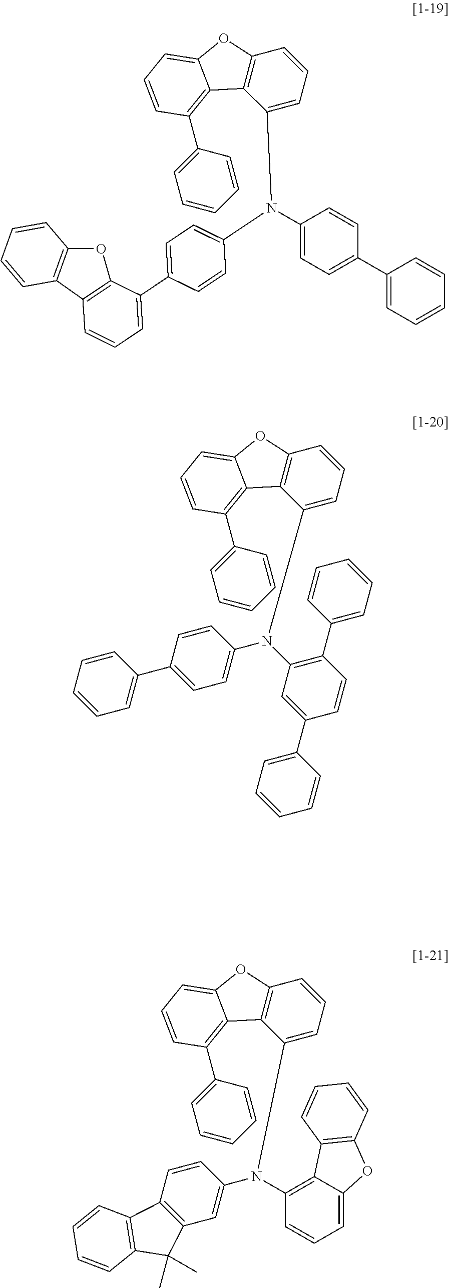 [SMILES: c1ccc(-c2ccc(N(c3ccc(-c4cccc5c4oc4ccccc45)cc3)c3cccc4oc5cccc(-c6ccccc6)c5c34)cc2)cc1 ; c1ccc(-c2ccc(N(c3cc(-c4ccccc4)ccc3-c3ccccc3)c3cccc4oc5cccc(-c6ccccc6)c5c34)cc2)cc1 ; CC1(C)c2ccccc2-c2ccc(N(c3cccc4oc5ccccc5c34)c3cccc4oc5cccc(-c6ccccc6)c5c34)cc21]

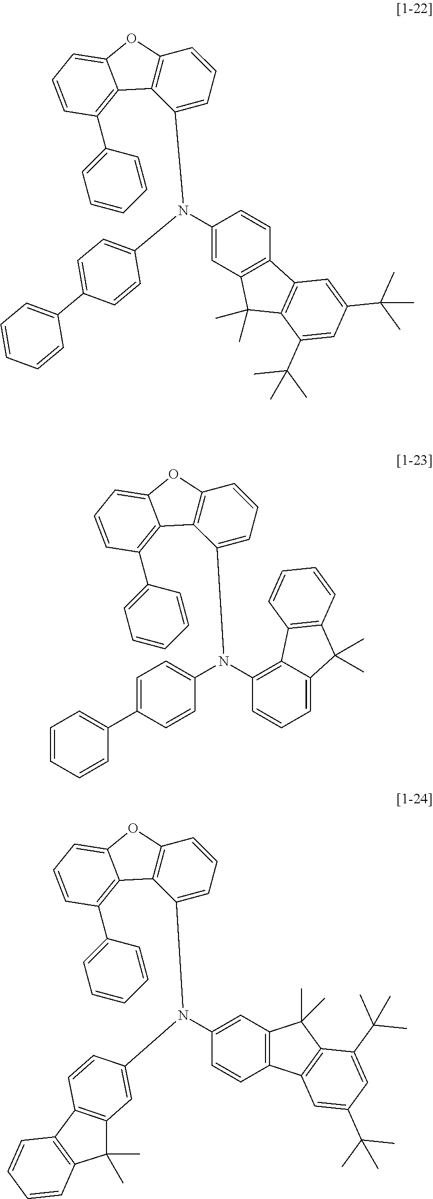 [SMILES: CC(C)(C)c1cc2c(c(C(C)(C)C)c1)C(C)(C)c1cc(N(c3ccc(-c4ccccc4)cc3)c3cccc4oc5cccc(-c6ccccc6)c5c34)ccc1-2 ; CC1(C)c2ccccc2-c2c(N(c3ccc(-c4ccccc4)cc3)c3cccc4oc5cccc(-c6ccccc6)c5c34)cccc21 ; CC(C)(C)c1cc2c(c(C(C)(C)C)c1)C(C)(C)c1cc(N(c3ccc4c(c3)C(C)(C)c3ccccc3-4)c3cccc4oc5cccc(-c6ccccc6)c5c34)ccc1-2]

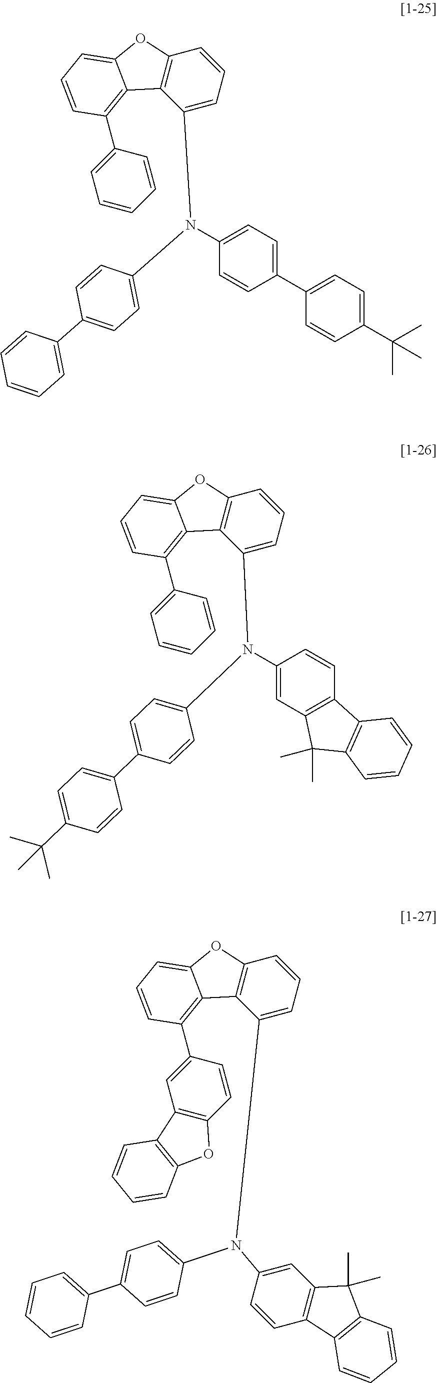

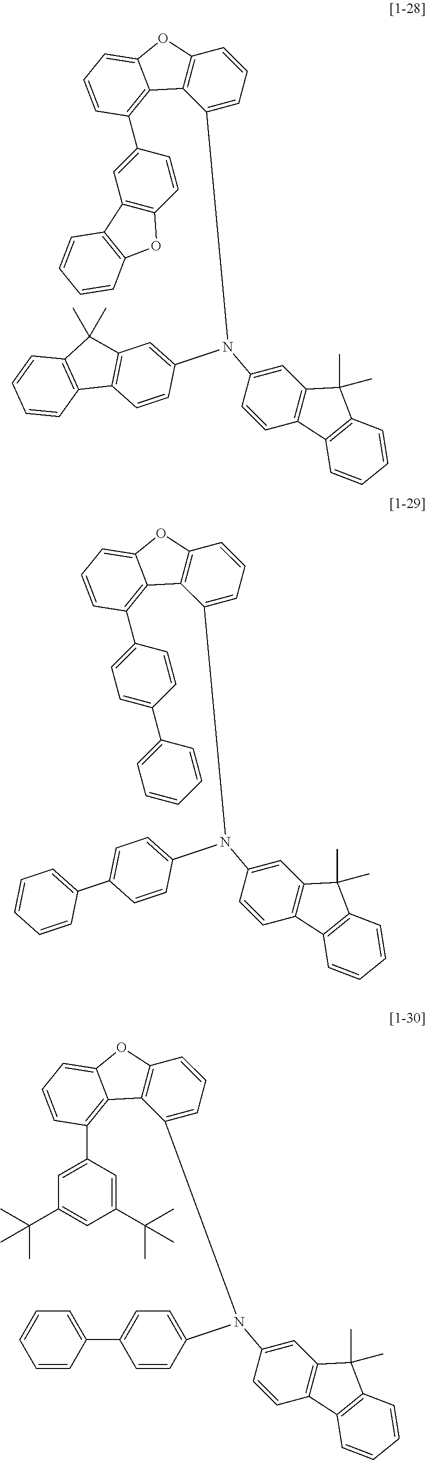 [SMILES: CC1(C)c2ccccc2-c2ccc(N(c3ccc4c(c3)C(C)(C)c3ccccc3-4)c3cccc4oc5cccc(-c6ccc7oc8ccccc8c7c6)c5c34)cc21 ; CC1(C)c2ccccc2-c2ccc(N(c3ccc(-c4ccccc4)cc3)c3cccc4oc5cccc(-c6ccc(-c7ccccc7)cc6)c5c34)cc21 ; CC(C)(C)c1cc(-c2cccc3oc4cccc(N(c5ccc(-c6ccccc6)cc5)c5ccc6c(c5)C(C)(C)c5ccccc5-6)c4c23)cc(C(C)(C)C)c1]

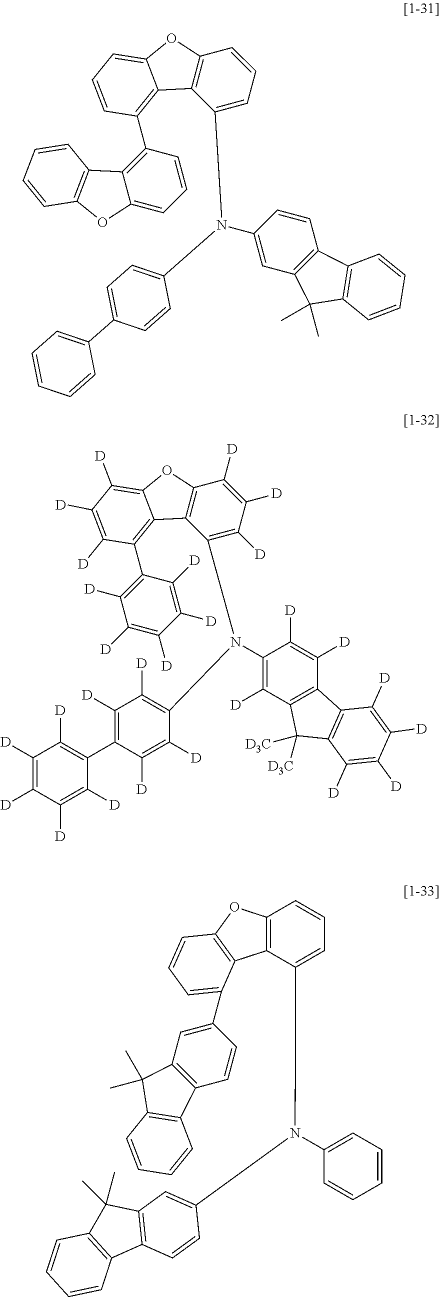 [SMILES: CC1(C)c2ccccc2-c2ccc(N(c3ccc(-c4ccccc4)cc3)c3cccc4oc5cccc(-c6cccc7oc8ccccc8c67)c5c34)cc21 ; [2H]c1c([2H])c([2H])c(-c2c([2H])c([2H])c(N(c3c([2H])c([2H])c4c(c3[2H])C(C([2H])([2H])[2H])(C([2H])([2H])[2H])c3c([2H])c([2H])c([2H])c([2H])c3-4)c3c([2H])c([2H])c([2H])c4oc5c([2H])c([2H])c([2H])c(-c6c([2H])c([2H])c([2H])c([2H])c6[2H])c5c34)c([2H])c2[2H])c([2H])c1[2H] ; CC1(C)c2ccccc2-c2ccc(-c3cccc4oc5cccc(N(c6ccccc6)c6ccc7c(c6)C(C)(C)c6ccccc6-7)c5c34)cc21]

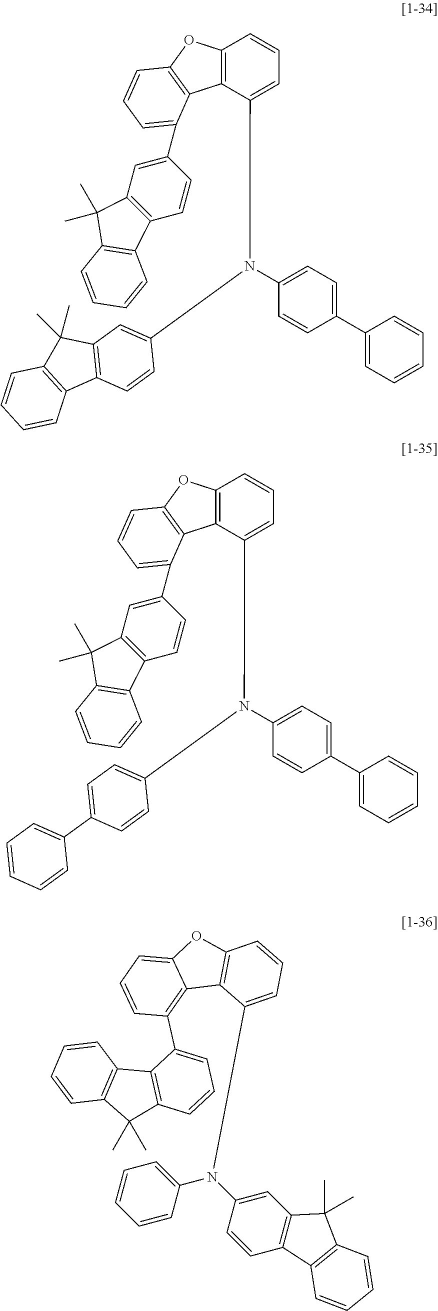 [SMILES: CC1(C)c2ccccc2-c2ccc(-c3cccc4oc5cccc(N(c6ccc(-c7ccccc7)cc6)c6ccc7c(c6)C(C)(C)c6ccccc6-7)c5c34)cc21 ; CC1(C)c2ccccc2-c2ccc(-c3cccc4oc5cccc(N(c6ccc(-c7ccccc7)cc6)c6ccc(-c7ccccc7)cc6)c5c34)cc21 ; CC1(C)c2ccccc2-c2ccc(N(c3ccccc3)c3cccc4oc5cccc(-c6cccc7c6-c6ccccc6C7(C)C)c5c34)cc21]

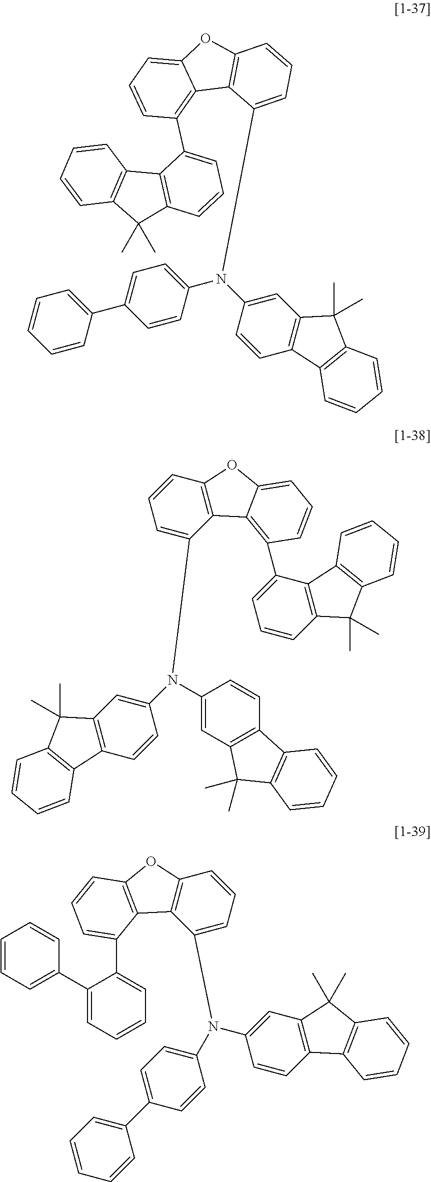 [SMILES: CC1(C)c2ccccc2-c2ccc(N(c3ccc(-c4ccccc4)cc3)c3cccc4oc5cccc(-c6cccc7c6-c6ccccc6C7(C)C)c5c34)cc21 ; CC1(C)c2ccccc2-c2ccc(N(c3ccc4c(c3)C(C)(C)c3ccccc3-4)c3cccc4oc5cccc(-c6cccc7c6-c6ccccc6C7(C)C)c5c34)cc21 ; CC1(C)c2ccccc2-c2ccc(N(c3ccc(-c4ccccc4)cc3)c3cccc4oc5cccc(-c6ccccc6-c6ccccc6)c5c34)cc21]

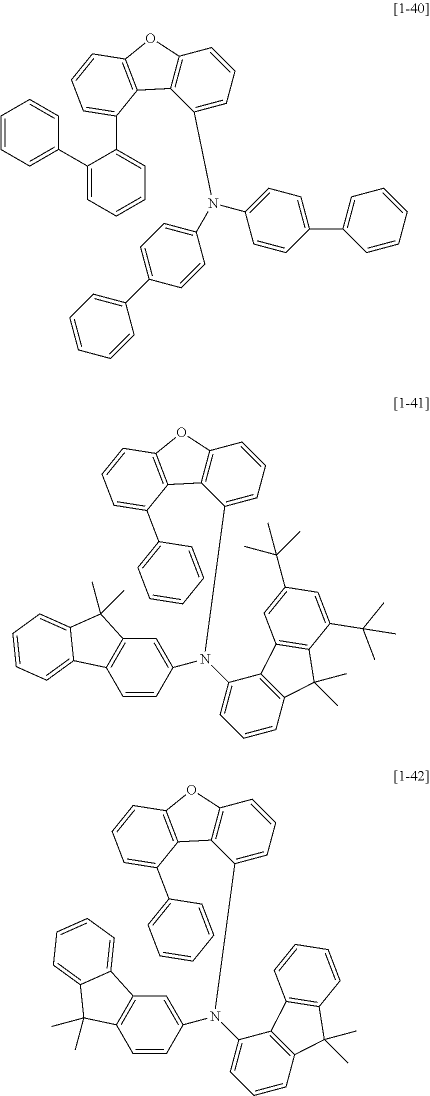

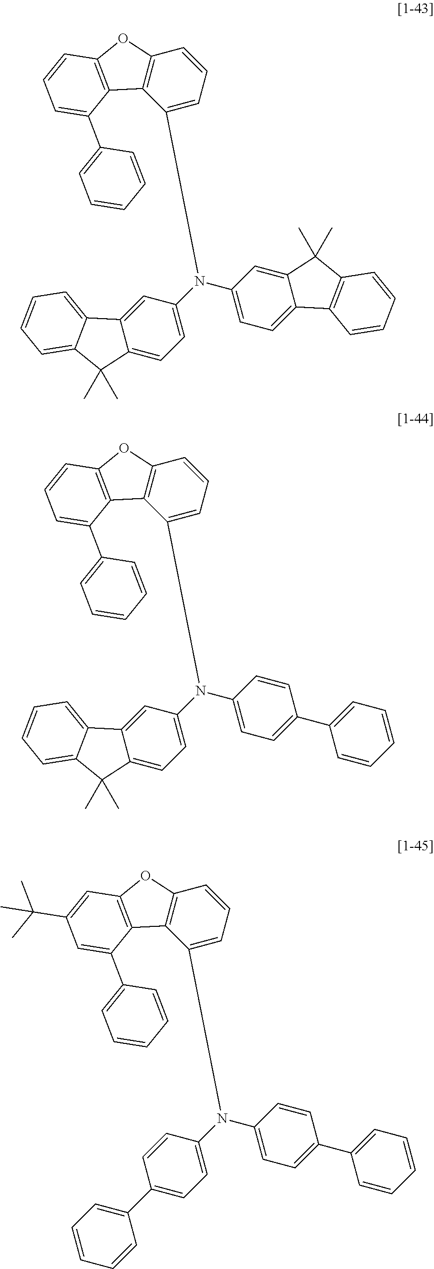 [SMILES: CC1(C)c2ccccc2-c2cc(N(c3ccc4c(c3)C(C)(C)c3ccccc3-4)c3cccc4oc5cccc(-c6ccccc6)c5c34)ccc21 ; CC1(C)c2ccccc2-c2cc(N(c3ccc(-c4ccccc4)cc3)c3cccc4oc5cccc(-c6ccccc6)c5c34)ccc21 ; CC(C)(C)c1cc(-c2ccccc2)c2c(c1)oc1cccc(N(c3ccc(-c4ccccc4)cc3)c3ccc(-c4ccccc4)cc3)c12]

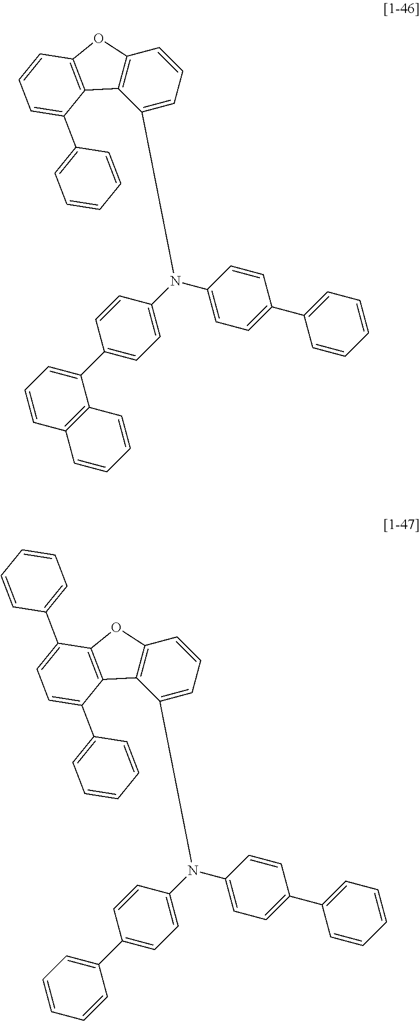 [SMILES: c1ccc(-c2ccc(N(c3ccc(-c4cccc5ccccc45)cc3)c3cccc4oc5cccc(-c6ccccc6)c5c34)cc2)cc1 ; c1ccc(-c2ccc(N(c3ccc(-c4ccccc4)cc3)c3cccc4oc5c(-c6ccccc6)ccc(-c6ccccc6)c5c34)cc2)cc1]

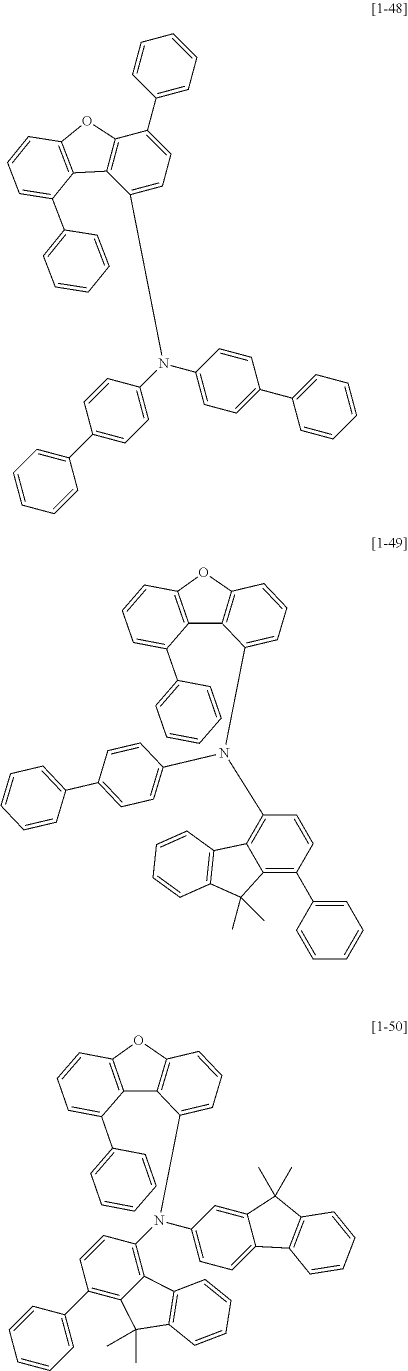 [SMILES: c1ccc(-c2ccc(N(c3ccc(-c4ccccc4)cc3)c3ccc(-c4ccccc4)c4oc5cccc(-c6ccccc6)c5c34)cc2)cc1 ; CC1(C)c2ccccc2-c2c(N(c3ccc(-c4ccccc4)cc3)c3cccc4oc5cccc(-c6ccccc6)c5c34)ccc(-c3ccccc3)c21 ; CC1(C)c2ccccc2-c2ccc(N(c3ccc(-c4ccccc4)c4c3-c3ccccc3C4(C)C)c3cccc4oc5cccc(-c6ccccc6)c5c34)cc21]

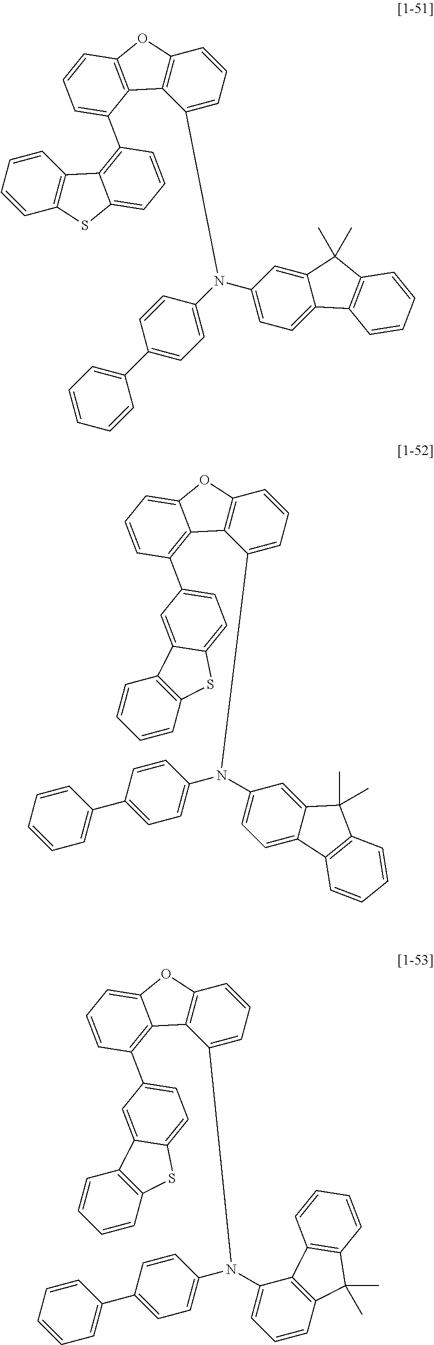 [SMILES: CC1(C)c2ccccc2-c2ccc(N(c3ccc(-c4ccccc4)cc3)c3cccc4oc5cccc(-c6cccc7sc8ccccc8c67)c5c34)cc21 ; CC1(C)c2ccccc2-c2ccc(N(c3ccc(-c4ccccc4)cc3)c3cccc4oc5cccc(-c6ccc7sc8ccccc8c7c6)c5c34)cc21 ; CC1(C)c2ccccc2-c2c(N(c3ccc(-c4ccccc4)cc3)c3cccc4oc5cccc(-c6ccc7sc8ccccc8c7c6)c5c34)cccc21]

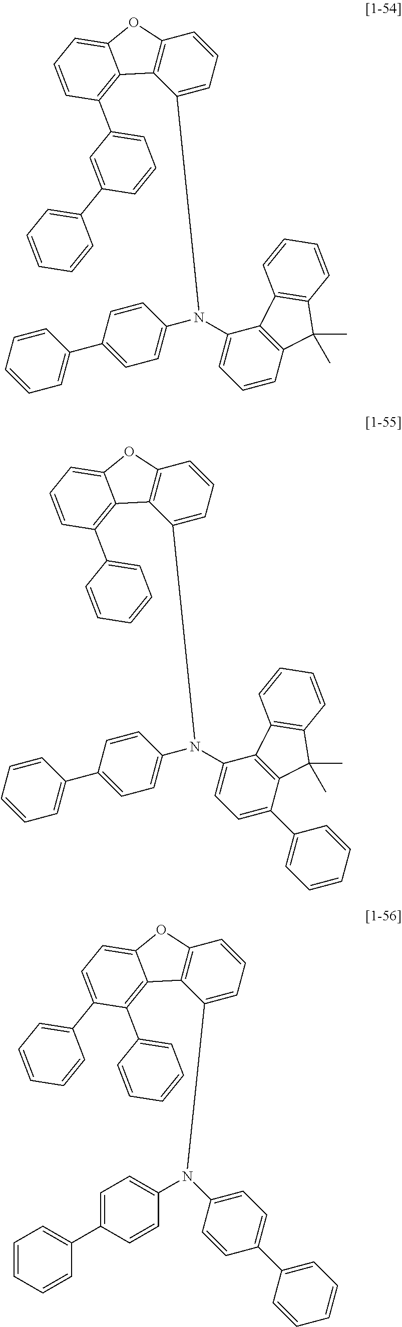 [SMILES: CC1(C)c2ccccc2-c2c(N(c3ccc(-c4ccccc4)cc3)c3cccc4oc5cccc(-c6cccc(-c7ccccc7)c6)c5c34)cccc21 ; CC1(C)c2ccccc2-c2c(N(c3ccc(-c4ccccc4)cc3)c3cccc4oc5cccc(-c6ccccc6)c5c34)ccc(-c3ccccc3)c21 ; c1ccc(-c2ccc(N(c3ccc(-c4ccccc4)cc3)c3cccc4oc5ccc(-c6ccccc6)c(-c6ccccc6)c5c34)cc2)cc1]

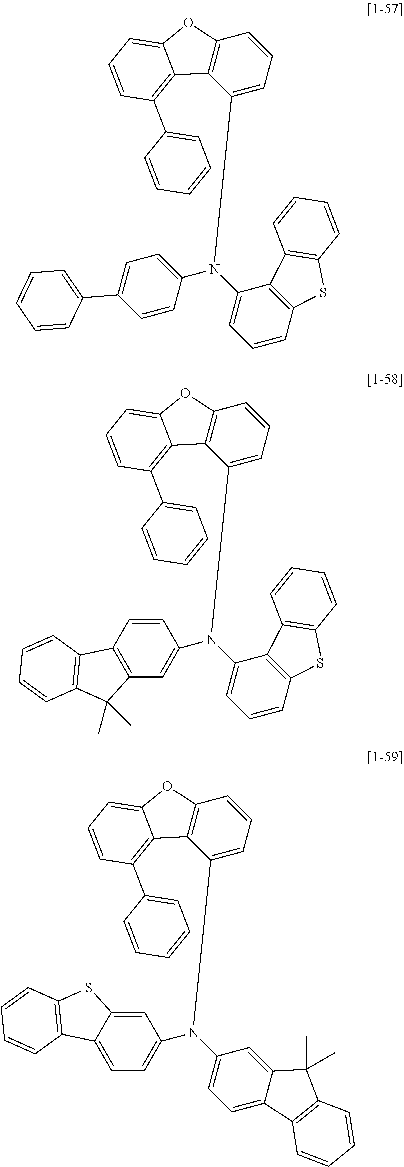 [SMILES: c1ccc(-c2ccc(N(c3cccc4sc5ccccc5c34)c3cccc4oc5cccc(-c6ccccc6)c5c34)cc2)cc1 ; CC1(C)c2ccccc2-c2ccc(N(c3cccc4sc5ccccc5c34)c3cccc4oc5cccc(-c6ccccc6)c5c34)cc21 ; CC1(C)c2ccccc2-c2ccc(N(c3ccc4c(c3)sc3ccccc34)c3cccc4oc5cccc(-c6ccccc6)c5c34)cc21]

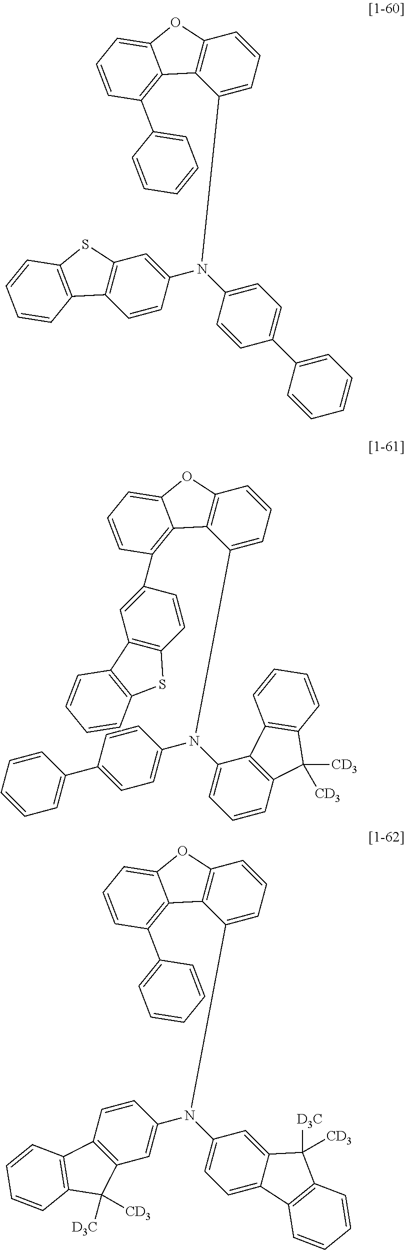

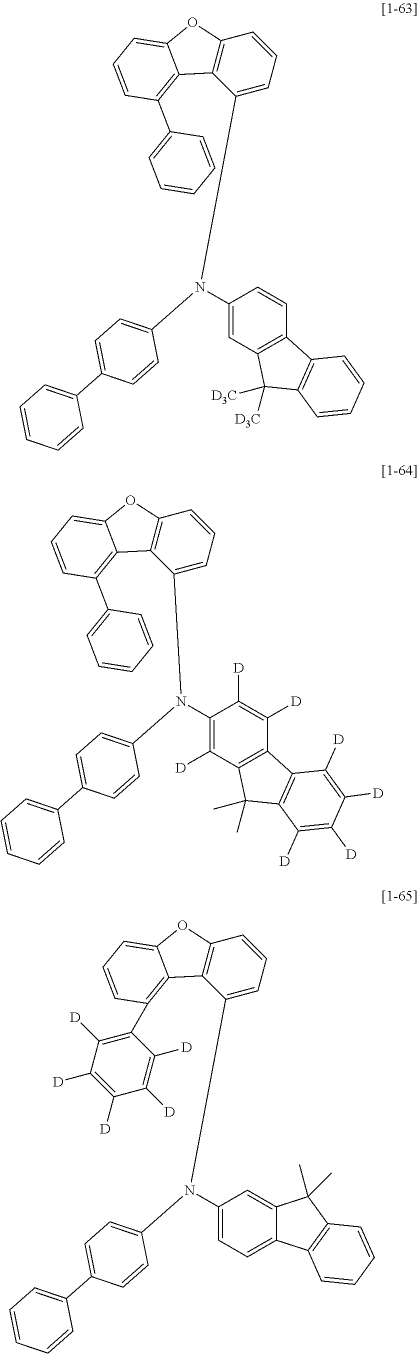 [SMILES: [2H]C([2H])([2H])C1(C([2H])([2H])[2H])c2ccccc2-c2ccc(N(c3ccc(-c4ccccc4)cc3)c3cccc4oc5cccc(-c6ccccc6)c5c34)cc21 ; [2H]c1c([2H])c([2H])c2c(c1[2H])-c1c([2H])c([2H])c(N(c3ccc(-c4ccccc4)cc3)c3cccc4oc5cccc(-c6ccccc6)c5c34)c([2H])c1C2(C)C ; [2H]c1c([2H])c([2H])c(-c2cccc3oc4cccc(N(c5ccc(-c6ccccc6)cc5)c5ccc6c(c5)C(C)(C)c5ccccc5-6)c4c23)c([2H])c1[2H]]

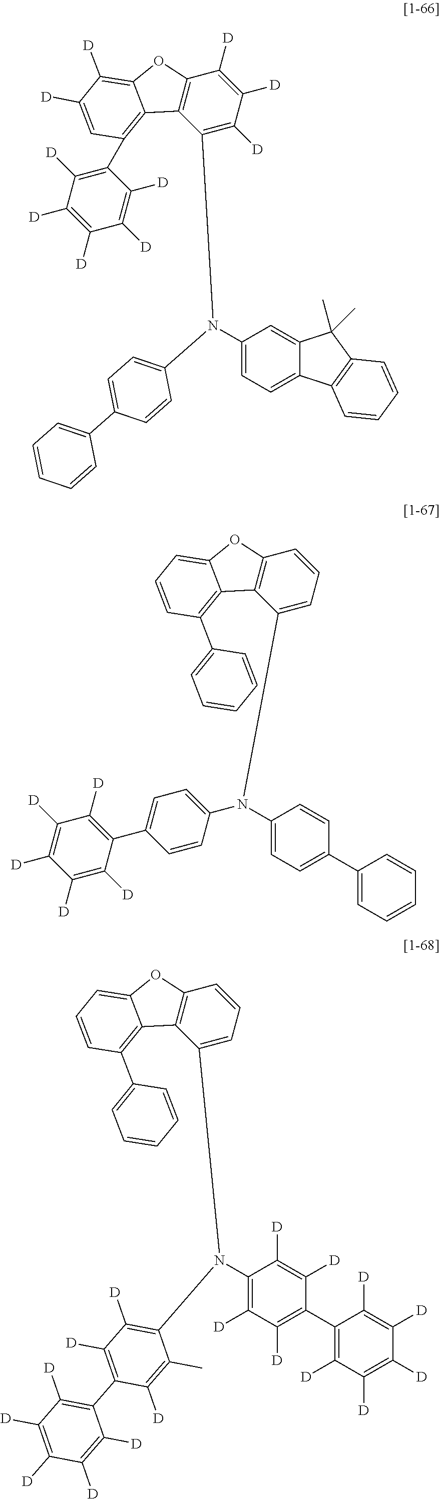 [SMILES: [2H]c1cc(-c2c([2H])c([2H])c([2H])c([2H])c2[2H])c2c(oc3c([2H])c([2H])c([2H])c(N(c4ccc(-c5ccccc5)cc4)c4ccc5c(c4)C(C)(C)c4ccccc4-5)c32)c1[2H] ; [2H]c1c([2H])c([2H])c(-c2ccc(N(c3ccc(-c4ccccc4)cc3)c3cccc4oc5cccc(-c6ccccc6)c5c34)cc2)c([2H])c1[2H] ; [2H]c1c([2H])c([2H])c(-c2c([2H])c([2H])c(N(c3c([2H])c([2H])c(-c4c([2H])c([2H])c([2H])c([2H])c4[2H])c([2H])c3C)c3cccc4oc5cccc(-c6ccccc6)c5c34)c([2H])c2[2H])c([2H])c1[2H]]

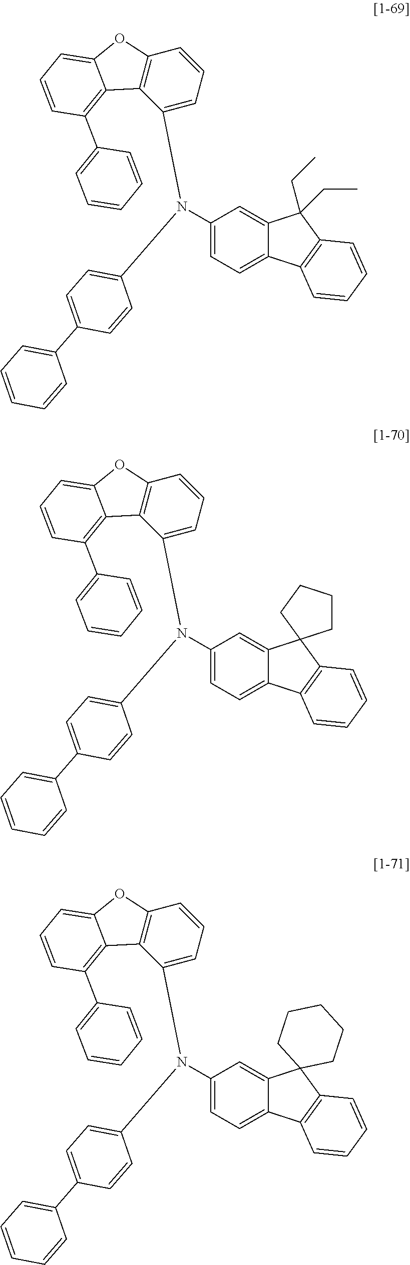 [SMILES: CCC1(CC)c2ccccc2-c2ccc(N(c3ccc(-c4ccccc4)cc3)c3cccc4oc5cccc(-c6ccccc6)c5c34)cc21 ; c1ccc(-c2ccc(N(c3ccc4c(c3)C3(CCCC3)c3ccccc3-4)c3cccc4oc5cccc(-c6ccccc6)c5c34)cc2)cc1 ; c1ccc(-c2ccc(N(c3ccc4c(c3)C3(CCCCC3)c3ccccc3-4)c3cccc4oc5cccc(-c6ccccc6)c5c34)cc2)cc1]

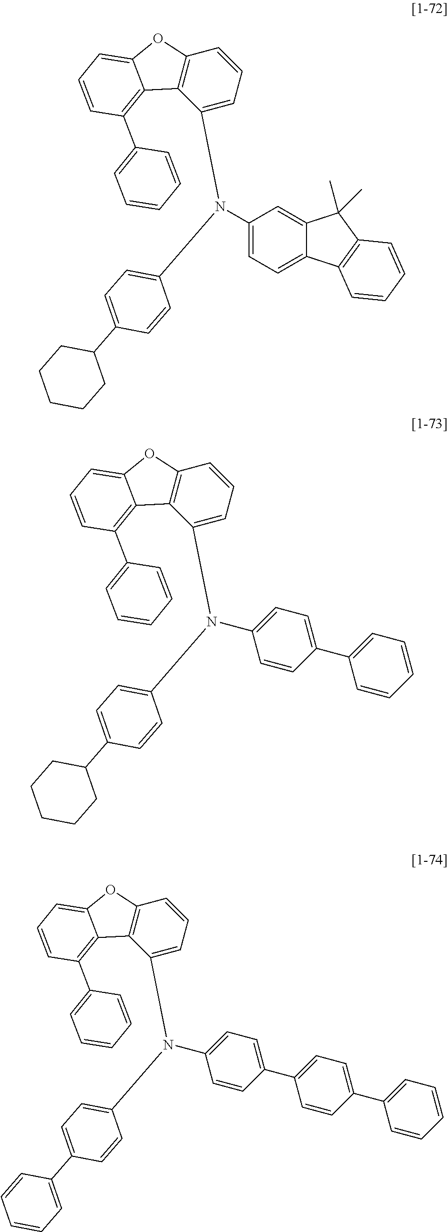

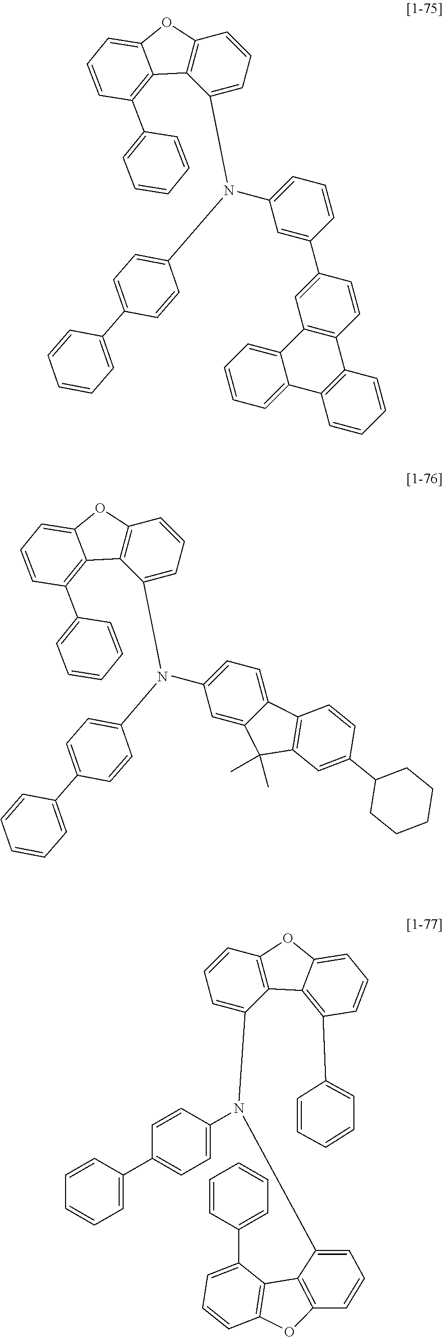

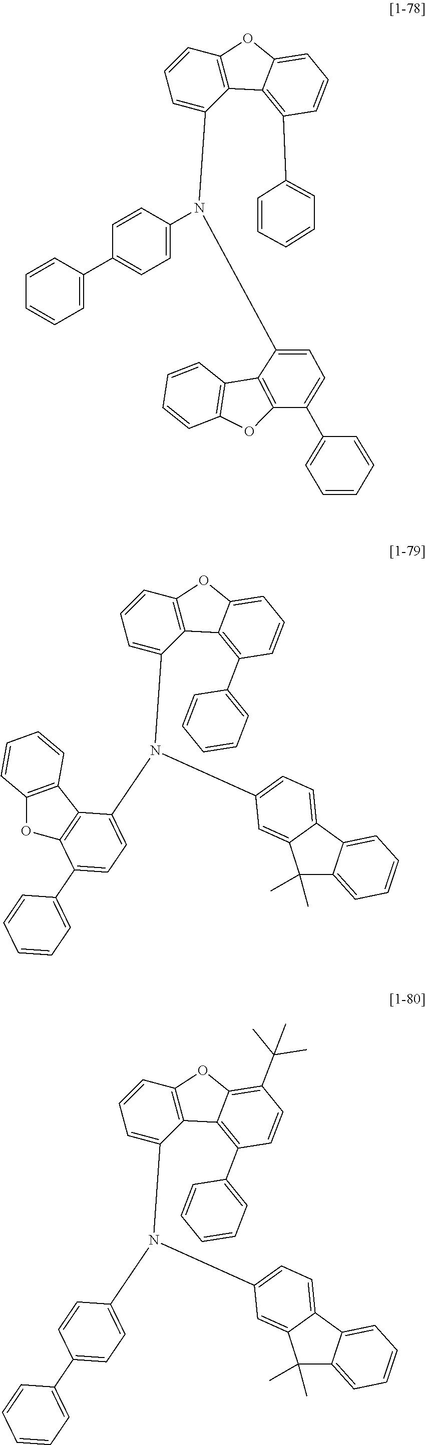 [SMILES: c1ccc(-c2ccc(N(c3ccc(-c4ccccc4)c4oc5ccccc5c34)c3cccc4oc5cccc(-c6ccccc6)c5c34)cc2)cc1 ; CC1(C)c2ccccc2-c2ccc(N(c3ccc(-c4ccccc4)c4oc5ccccc5c34)c3cccc4oc5cccc(-c6ccccc6)c5c34)cc21 ; CC(C)(C)c1ccc(-c2ccccc2)c2c1oc1cccc(N(c3ccc(-c4ccccc4)cc3)c3ccc4c(c3)C(C)(C)c3ccccc3-4)c12]

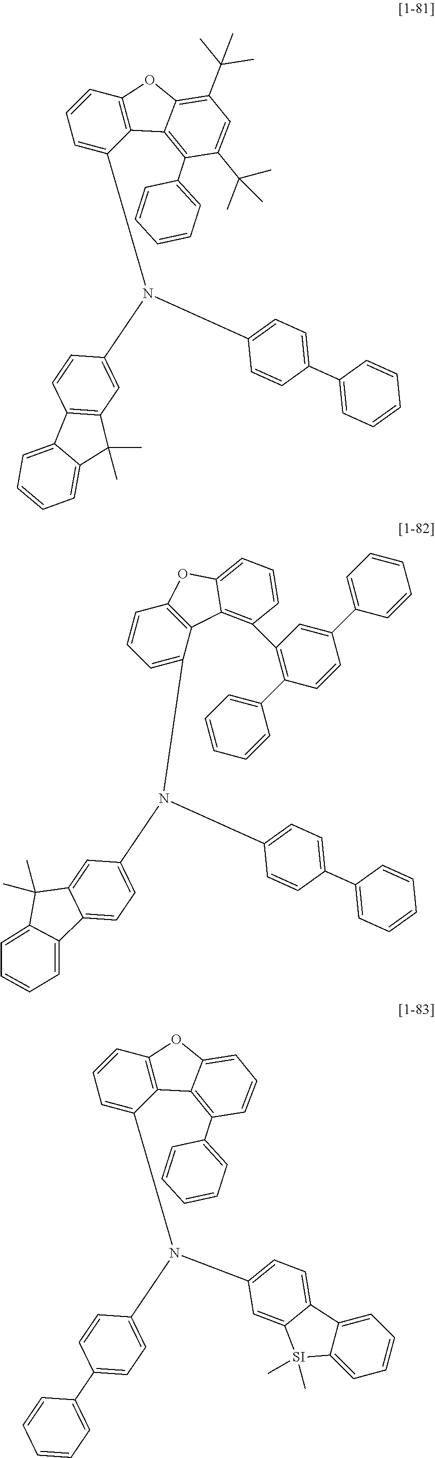

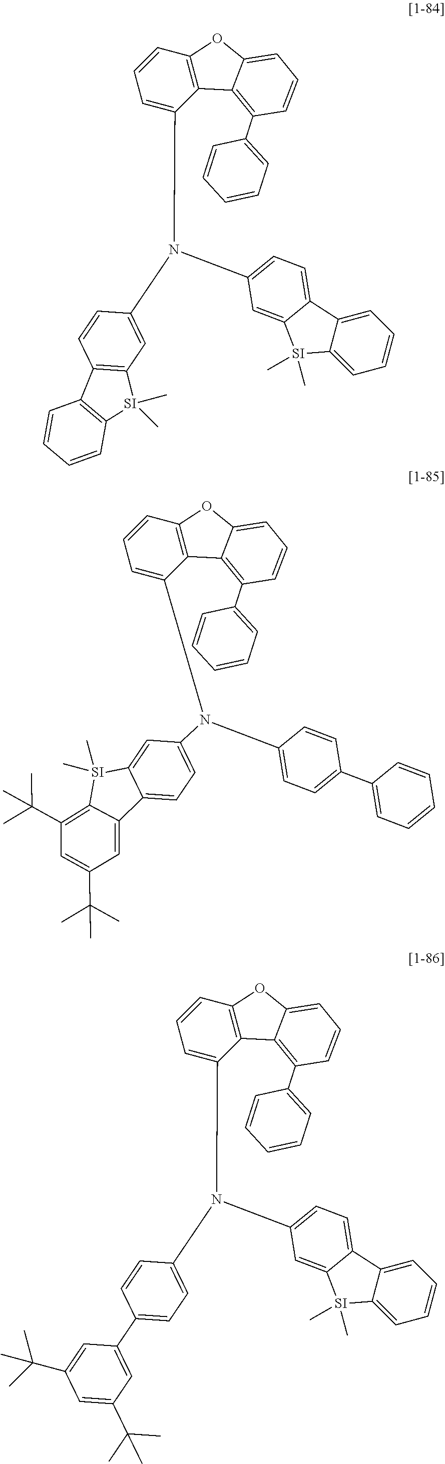

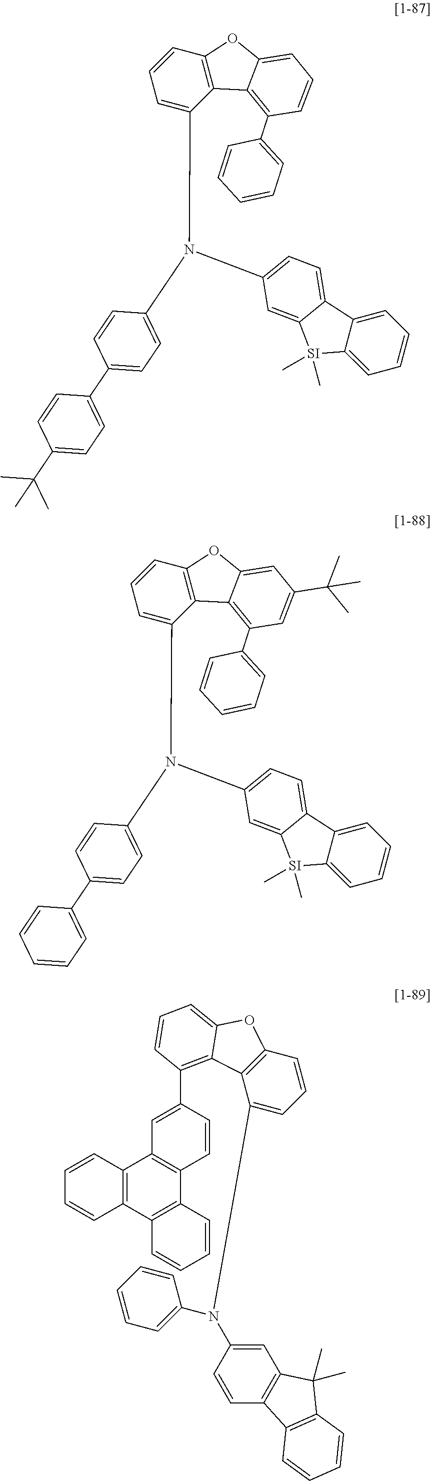

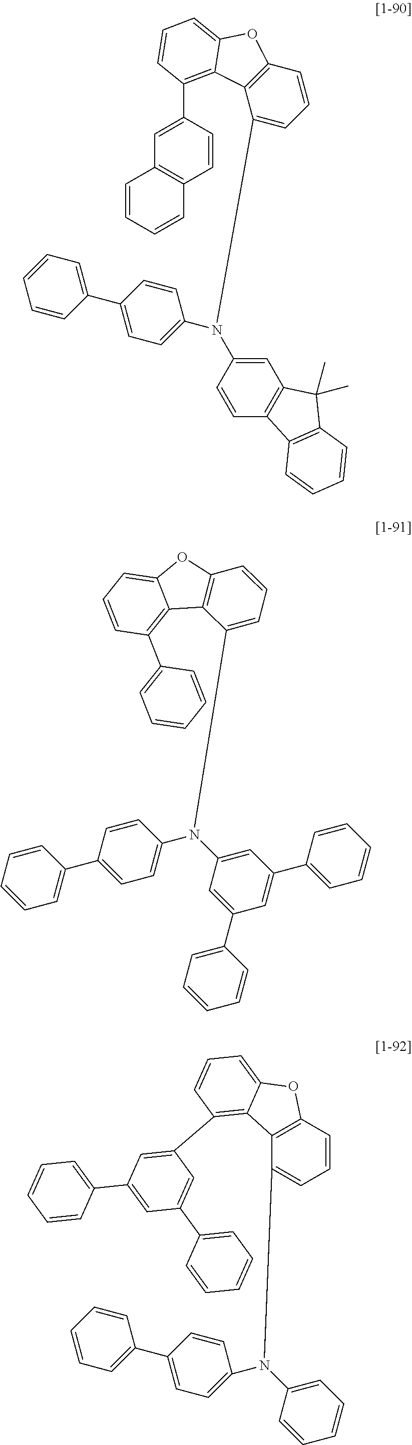 [SMILES: CC1(C)c2ccccc2-c2ccc(N(c3ccc(-c4ccccc4)cc3)c3cccc4oc5cccc(-c6ccc7ccccc7c6)c5c34)cc21 ; c1ccc(-c2ccc(N(c3cc(-c4ccccc4)cc(-c4ccccc4)c3)c3cccc4oc5cccc(-c6ccccc6)c5c34)cc2)cc1 ; c1ccc(-c2ccc(N(c3ccccc3)c3cccc4oc5cccc(-c6cc(-c7ccccc7)cc(-c7ccccc7)c6)c5c34)cc2)cc1]

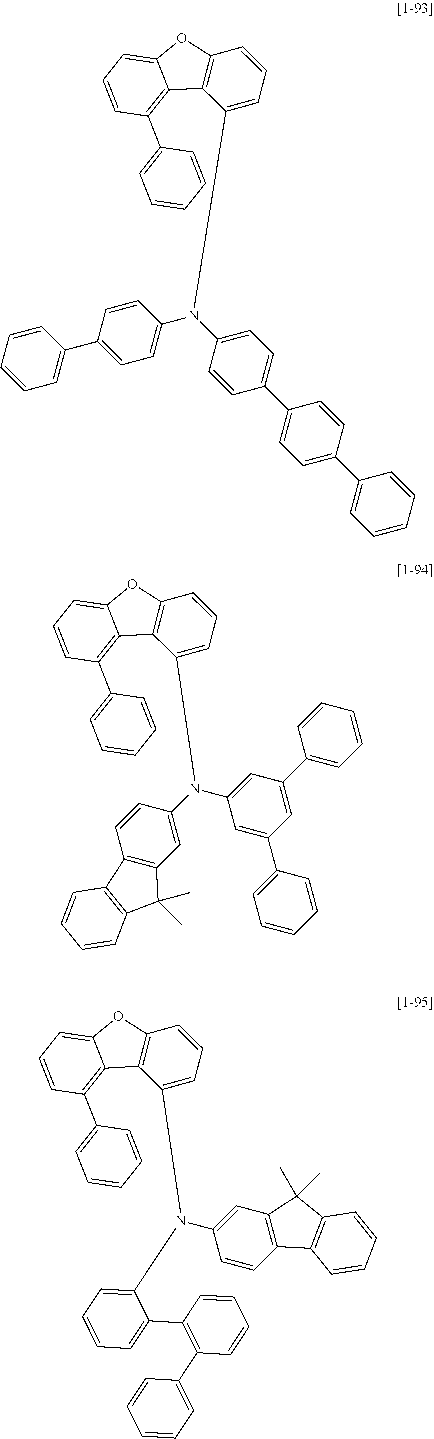 [SMILES: c1ccc(-c2ccc(-c3ccc(N(c4ccc(-c5ccccc5)cc4)c4cccc5oc6cccc(-c7ccccc7)c6c45)cc3)cc2)cc1 ; CC1(C)c2ccccc2-c2ccc(N(c3cc(-c4ccccc4)cc(-c4ccccc4)c3)c3cccc4oc5cccc(-c6ccccc6)c5c34)cc21 ; CC1(C)c2ccccc2-c2ccc(N(c3ccccc3-c3ccccc3-c3ccccc3)c3cccc4oc5cccc(-c6ccccc6)c5c34)cc21]

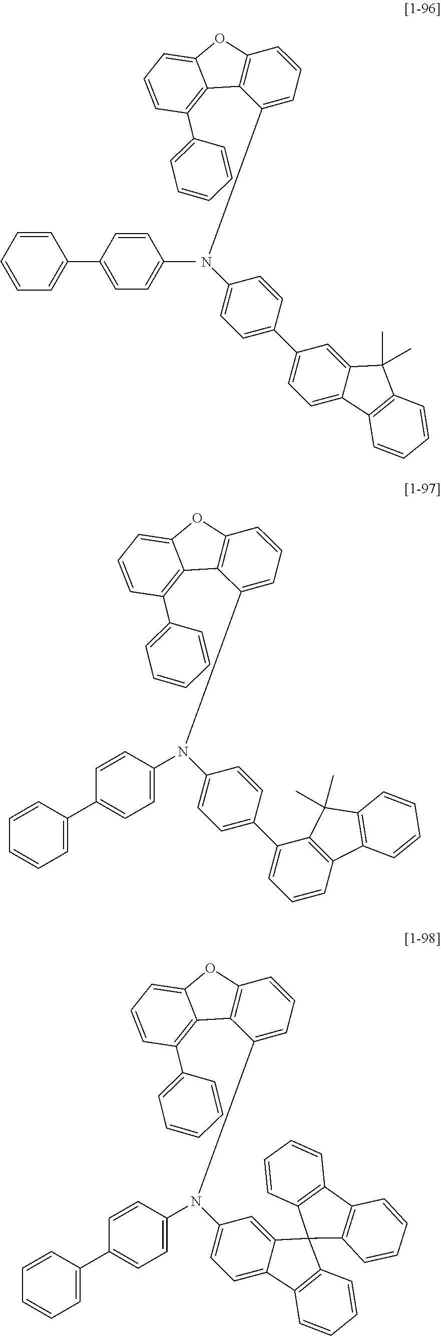

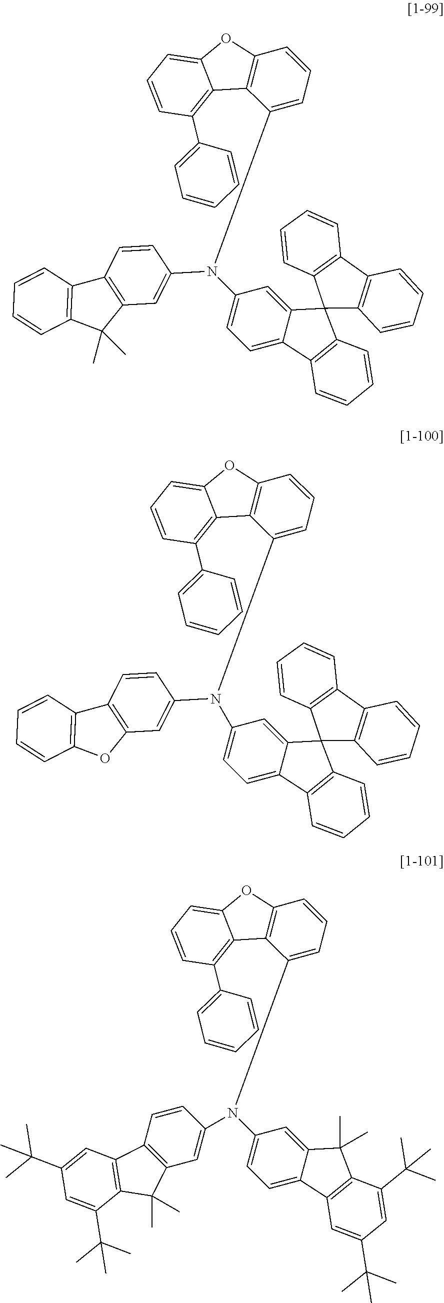 [SMILES: CC1(C)c2ccccc2-c2ccc(N(c3ccc4c(c3)C3(c5ccccc5-c5ccccc53)c3ccccc3-4)c3cccc4oc5cccc(-c6ccccc6)c5c34)cc21 ; c1ccc(-c2cccc3oc4cccc(N(c5ccc6c(c5)C5(c7ccccc7-c7ccccc75)c5ccccc5-6)c5ccc6c(c5)oc5ccccc56)c4c23)cc1 ; CC(C)(C)c1cc2c(c(C(C)(C)C)c1)C(C)(C)c1cc(N(c3ccc4c(c3)C(C)(C)c3c-4cc(C(C)(C)C)cc3C(C)(C)C)c3cccc4oc5cccc(-c6ccccc6)c5c34)ccc1-2]

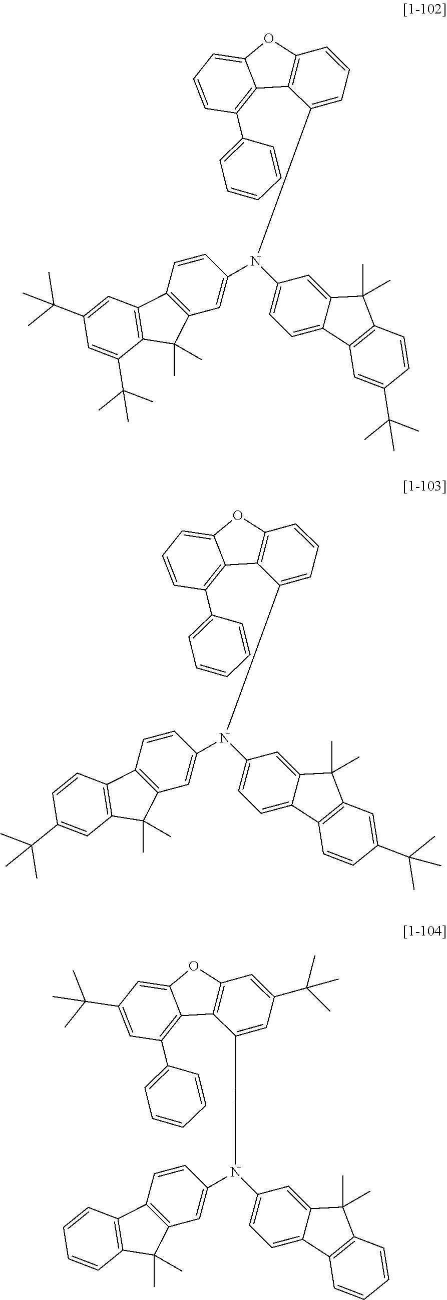

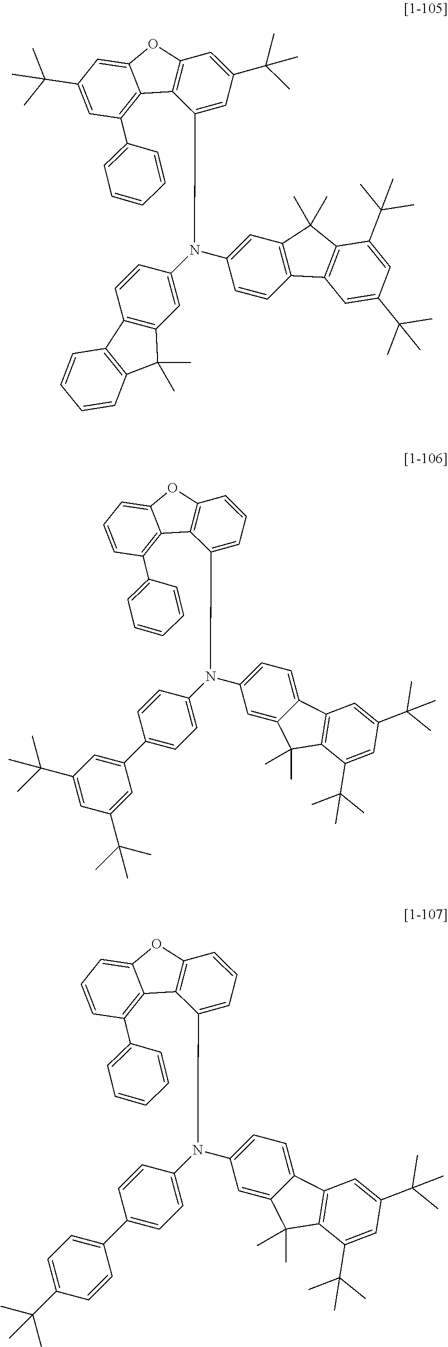

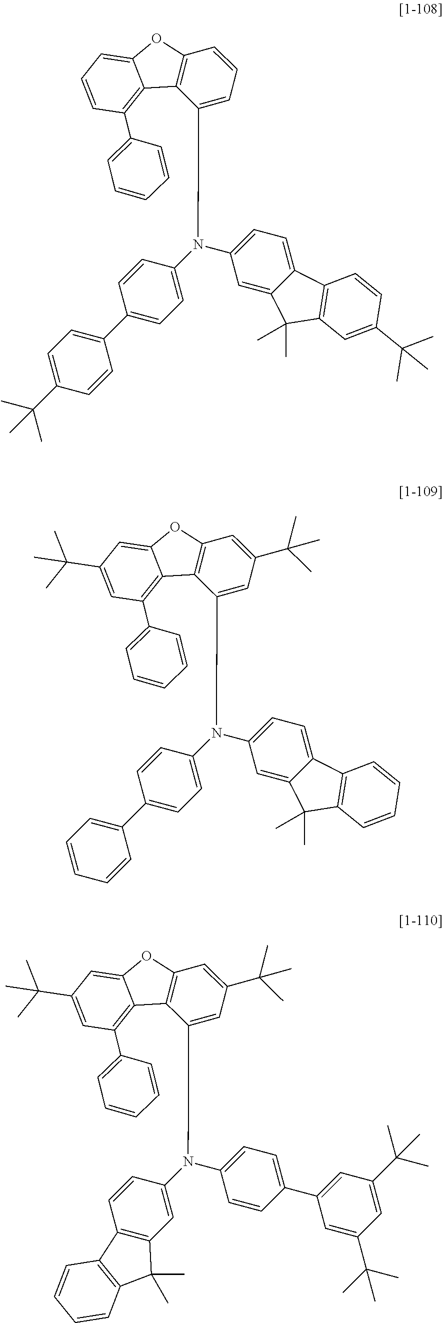

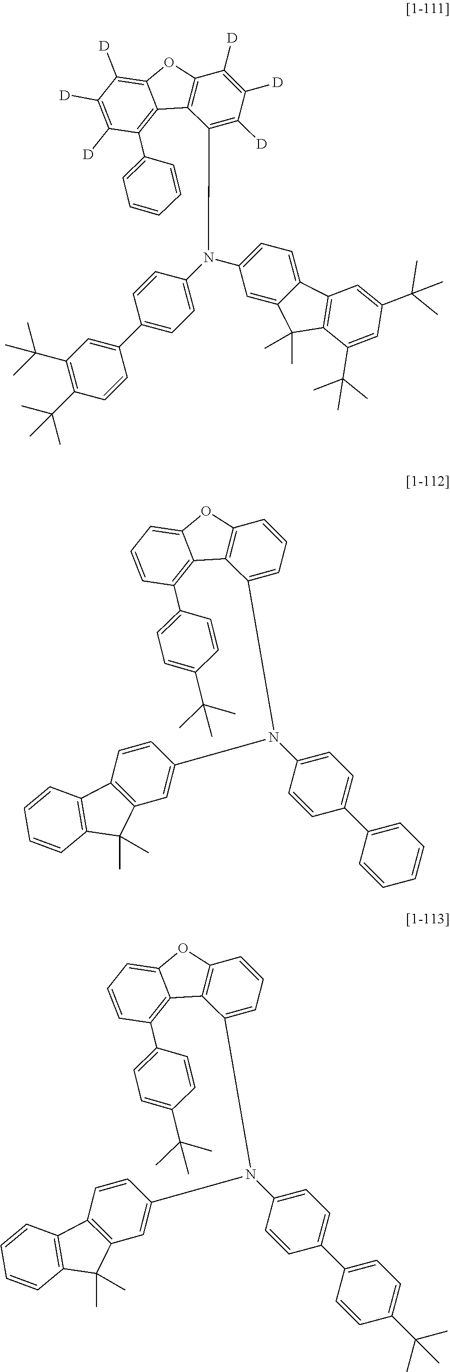 [SMILES: [2H]c1c([2H])c(-c2ccccc2)c2c(oc3c([2H])c([2H])c([2H])c(N(c4ccc(-c5ccc(C(C)(C)C)c(C(C)(C)C)c5)cc4)c4ccc5c(c4)C(C)(C)c4c-5cc(C(C)(C)C)cc4C(C)(C)C)c32)c1[2H] ; CC(C)(C)c1ccc(-c2cccc3oc4cccc(N(c5ccc(-c6ccccc6)cc5)c5ccc6c(c5)C(C)(C)c5ccccc5-6)c4c23)cc1 ; CC(C)(C)c1ccc(-c2ccc(N(c3ccc4c(c3)C(C)(C)c3ccccc3-4)c3cccc4oc5cccc(-c6ccc(C(C)(C)C)cc6)c5c34)cc2)cc1]

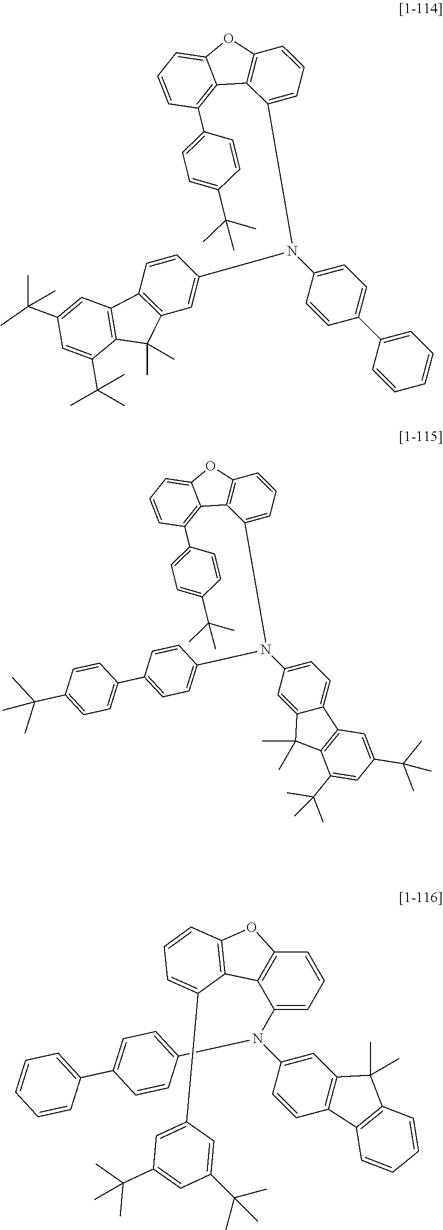 [SMILES: CC(C)(C)c1ccc(-c2cccc3oc4cccc(N(c5ccc(-c6ccccc6)cc5)c5ccc6c(c5)C(C)(C)c5c-6cc(C(C)(C)C)cc5C(C)(C)C)c4c23)cc1 ; CC(C)(C)c1ccc(-c2ccc(N(c3ccc4c(c3)C(C)(C)c3c-4cc(C(C)(C)C)cc3C(C)(C)C)c3cccc4oc5cccc(-c6ccc(C(C)(C)C)cc6)c5c34)cc2)cc1 ; CC(C)(C)c1cc(-c2cccc3oc4cccc(N(c5ccc(-c6ccccc6)cc5)c5ccc6c(c5)C(C)(C)c5ccccc5-6)c4c23)cc(C(C)(C)C)c1]

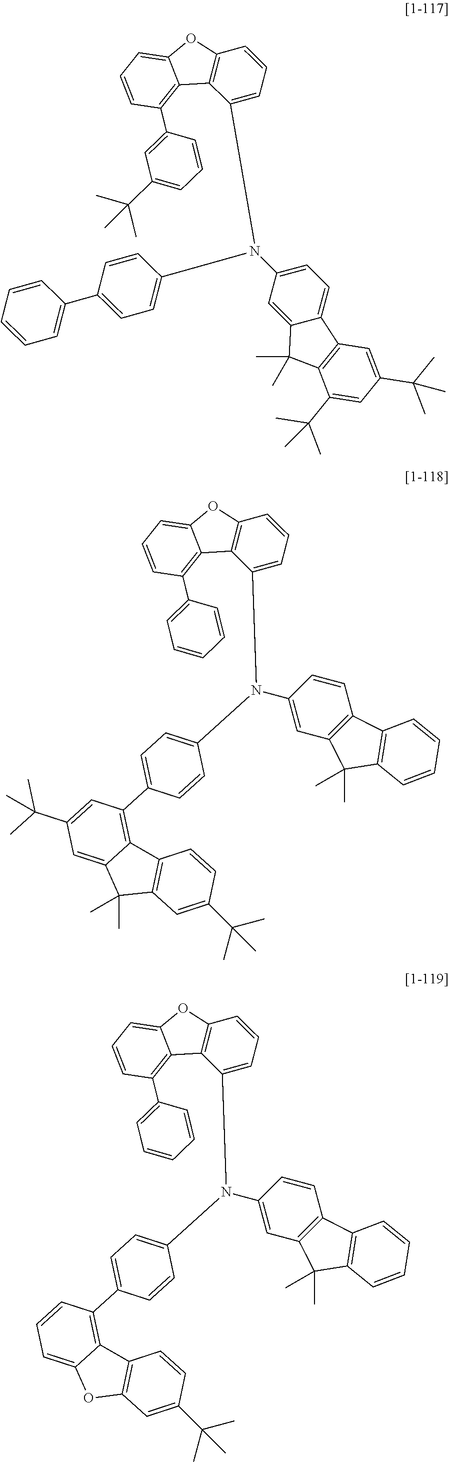

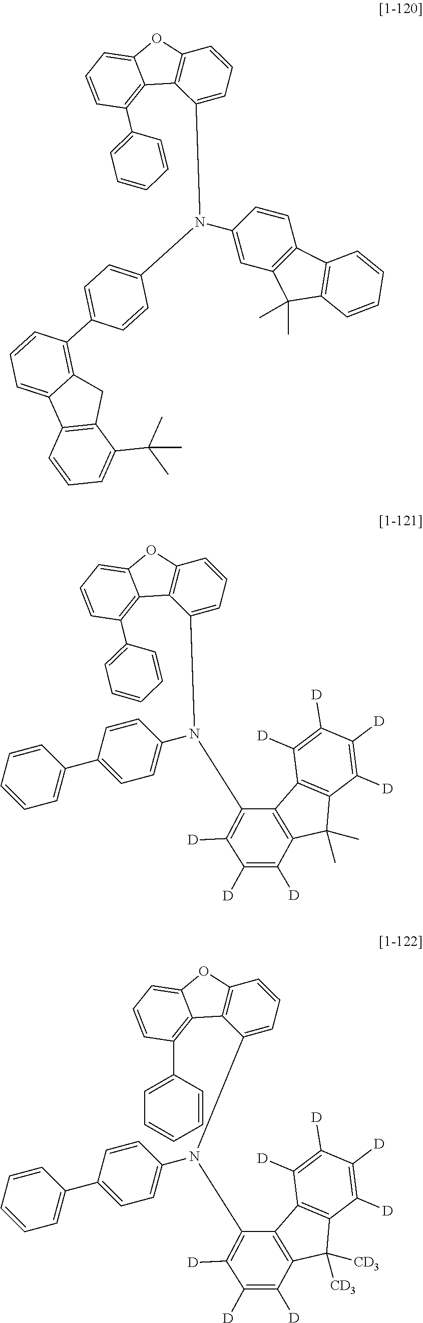 [SMILES: CC(C)(C)c1cccc2c1Cc1c(-c3ccc(N(c4ccc5c(c4)C(C)(C)c4ccccc4-5)c4cccc5oc6cccc(-c7ccccc7)c6c45)cc3)cccc1-2 ; [2H]c1c([2H])c([2H])c2c(c1[2H])-c1c(N(c3ccc(-c4ccccc4)cc3)c3cccc4oc5cccc(-c6ccccc6)c5c34)c([2H])c([2H])c([2H])c1C2(C)C ; [2H]c1c([2H])c([2H])c2c(c1[2H])-c1c(N(c3ccc(-c4ccccc4)cc3)c3cccc4oc5cccc(-c6ccccc6)c5c34)c([2H])c([2H])c([2H])c1C2(C([2H])([2H])[2H])C([2H])([2H])[2H]]

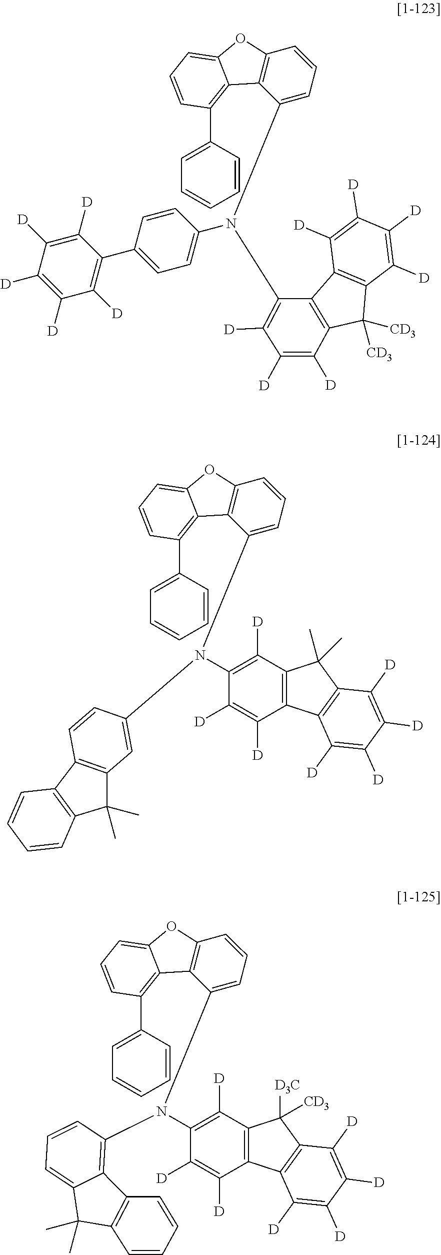 [SMILES: [2H]c1c([2H])c([2H])c(-c2ccc(N(c3c([2H])c([2H])c([2H])c4c3-c3c([2H])c([2H])c([2H])c([2H])c3C4(C([2H])([2H])[2H])C([2H])([2H])[2H])c3cccc4oc5cccc(-c6ccccc6)c5c34)cc2)c([2H])c1[2H] ; [2H]c1c([2H])c([2H])c2c(c1[2H])-c1c([2H])c([2H])c(N(c3ccc4c(c3)C(C)(C)c3ccccc3-4)c3cccc4oc5cccc(-c6ccccc6)c5c34)c([2H])c1C2(C)C ; [2H]c1c([2H])c([2H])c2c(c1[2H])-c1c([2H])c([2H])c(N(c3cccc4c3-c3ccccc3C4(C)C)c3cccc4oc5cccc(-c6ccccc6)c5c34)c([2H])c1C2(C([2H])([2H])[2H])C([2H])([2H])[2H]]

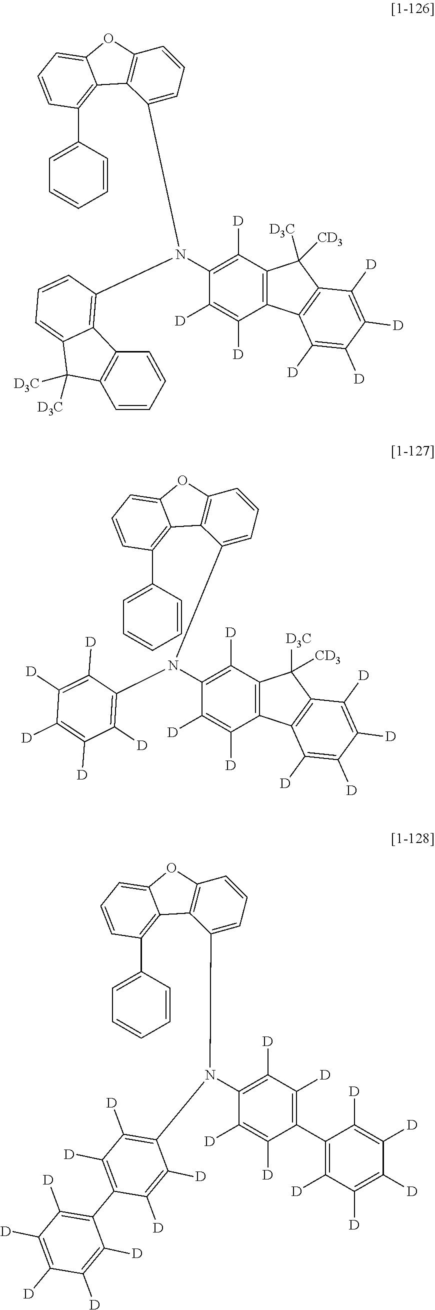

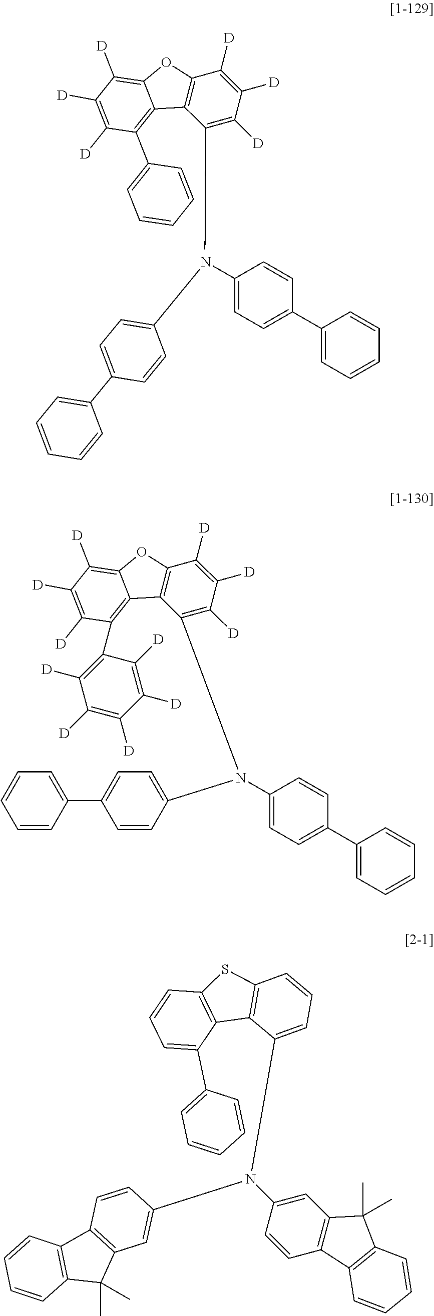

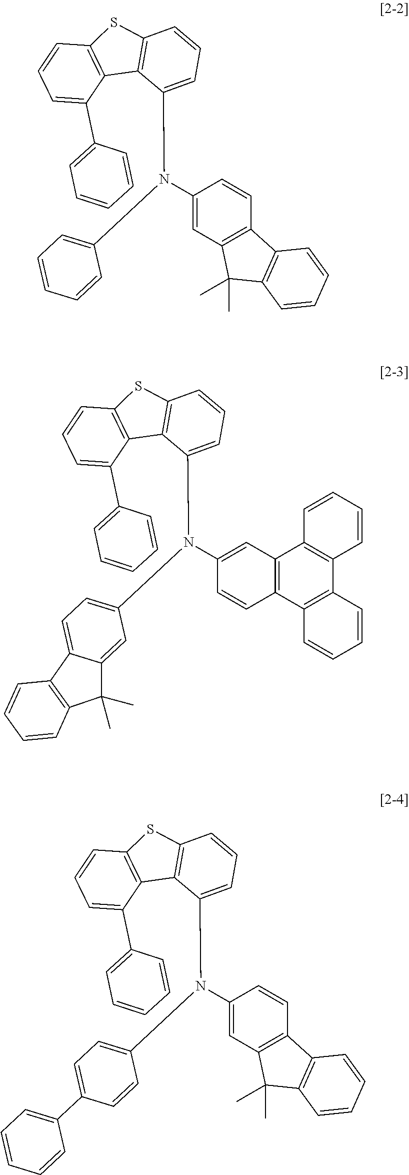 [SMILES: CC1(C)c2ccccc2-c2ccc(N(c3ccccc3)c3cccc4sc5cccc(-c6ccccc6)c5c34)cc21 ; CC1(C)c2ccccc2-c2ccc(N(c3ccc4c5ccccc5c5ccccc5c4c3)c3cccc4sc5cccc(-c6ccccc6)c5c34)cc21 ; CC1(C)c2ccccc2-c2ccc(N(c3ccc(-c4ccccc4)cc3)c3cccc4sc5cccc(-c6ccccc6)c5c34)cc21]

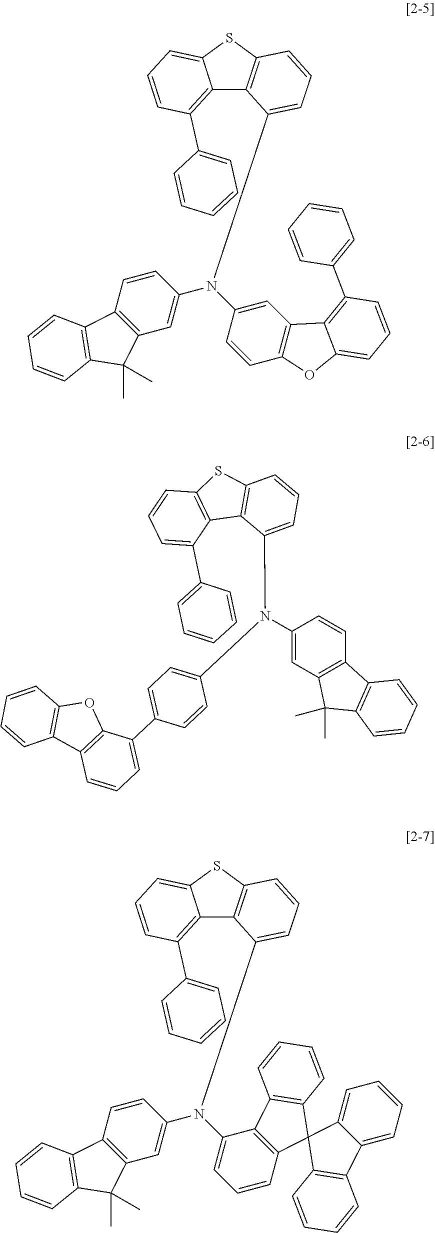 [SMILES: CC1(C)c2ccccc2-c2ccc(N(c3ccc4oc5cccc(-c6ccccc6)c5c4c3)c3cccc4sc5cccc(-c6ccccc6)c5c34)cc21 ; CC1(C)c2ccccc2-c2ccc(N(c3ccc(-c4cccc5c4oc4ccccc45)cc3)c3cccc4sc5cccc(-c6ccccc6)c5c34)cc21 ; CC1(C)c2ccccc2-c2ccc(N(c3cccc4c3-c3ccccc3C43c4ccccc4-c4ccccc43)c3cccc4sc5cccc(-c6ccccc6)c5c34)cc21]

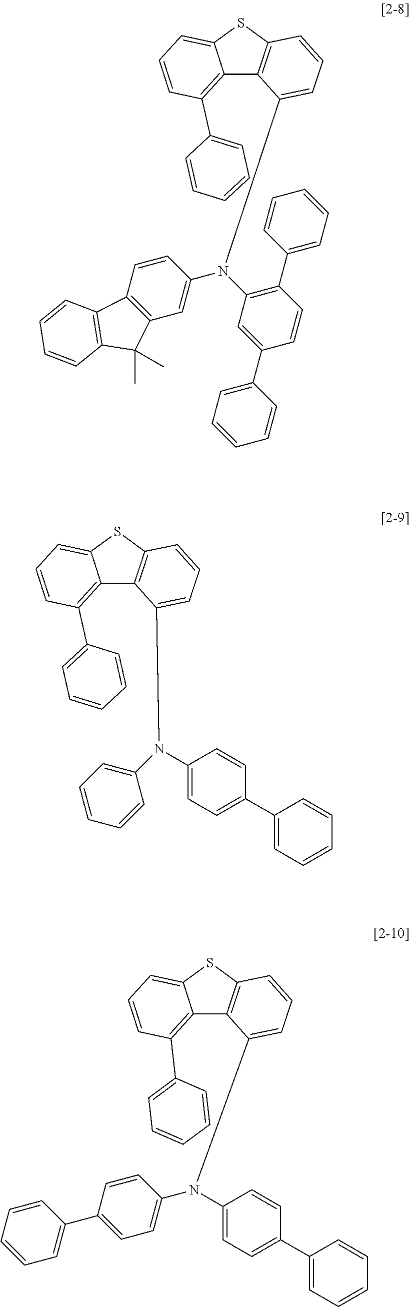 [SMILES: CC1(C)c2ccccc2-c2ccc(N(c3cc(-c4ccccc4)ccc3-c3ccccc3)c3cccc4sc5cccc(-c6ccccc6)c5c34)cc21 ; c1ccc(-c2ccc(N(c3ccccc3)c3cccc4sc5cccc(-c6ccccc6)c5c34)cc2)cc1 ; c1ccc(-c2ccc(N(c3ccc(-c4ccccc4)cc3)c3cccc4sc5cccc(-c6ccccc6)c5c34)cc2)cc1]

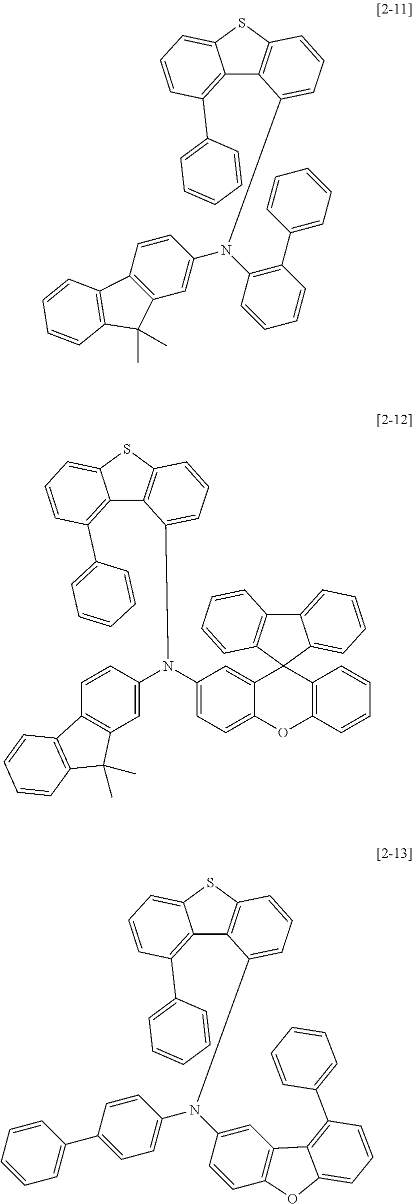 [SMILES: CC1(C)c2ccccc2-c2ccc(N(c3ccccc3-c3ccccc3)c3cccc4sc5cccc(-c6ccccc6)c5c34)cc21 ; CC1(C)c2ccccc2-c2ccc(N(c3ccc4c(c3)C3(c5ccccc5O4)c4ccccc4-c4ccccc43)c3cccc4sc5cccc(-c6ccccc6)c5c34)cc21 ; c1ccc(-c2ccc(N(c3ccc4oc5cccc(-c6ccccc6)c5c4c3)c3cccc4sc5cccc(-c6ccccc6)c5c34)cc2)cc1]

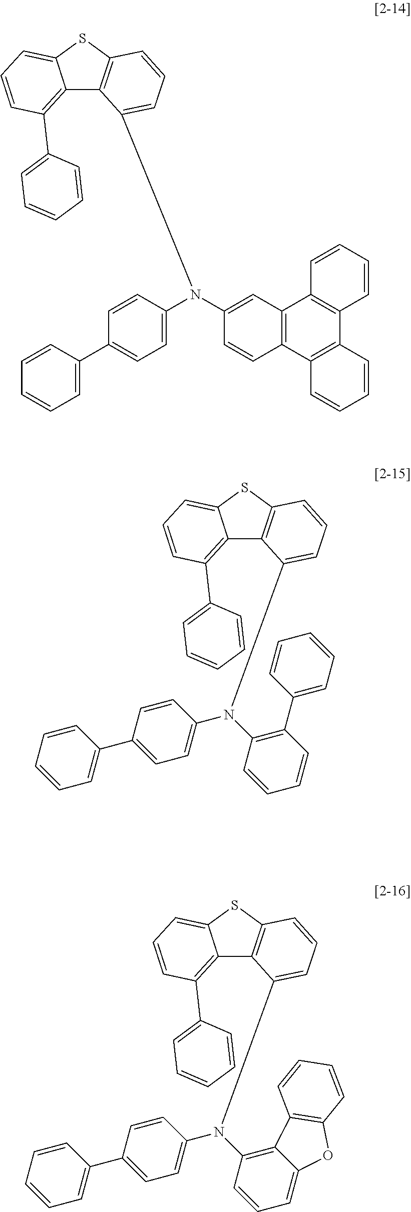 [SMILES: c1ccc(-c2ccc(N(c3ccc4c5ccccc5c5ccccc5c4c3)c3cccc4sc5cccc(-c6ccccc6)c5c34)cc2)cc1 ; c1ccc(-c2ccc(N(c3ccccc3-c3ccccc3)c3cccc4sc5cccc(-c6ccccc6)c5c34)cc2)cc1 ; c1ccc(-c2ccc(N(c3cccc4oc5ccccc5c34)c3cccc4sc5cccc(-c6ccccc6)c5c34)cc2)cc1]

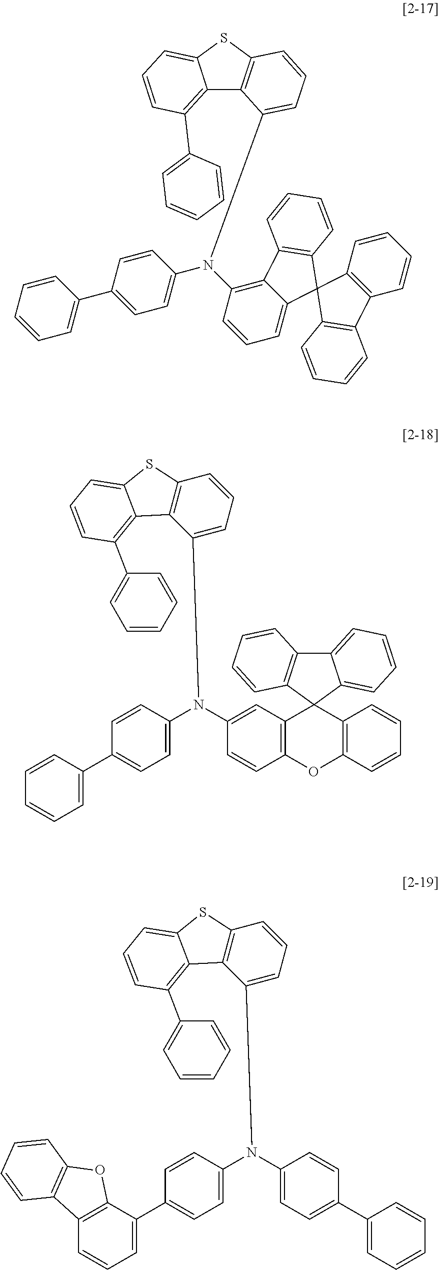 [SMILES: c1ccc(-c2ccc(N(c3cccc4c3-c3ccccc3C43c4ccccc4-c4ccccc43)c3cccc4sc5cccc(-c6ccccc6)c5c34)cc2)cc1 ; c1ccc(-c2ccc(N(c3ccc4c(c3)C3(c5ccccc5O4)c4ccccc4-c4ccccc43)c3cccc4sc5cccc(-c6ccccc6)c5c34)cc2)cc1 ; c1ccc(-c2ccc(N(c3ccc(-c4cccc5c4oc4ccccc45)cc3)c3cccc4sc5cccc(-c6ccccc6)c5c34)cc2)cc1]

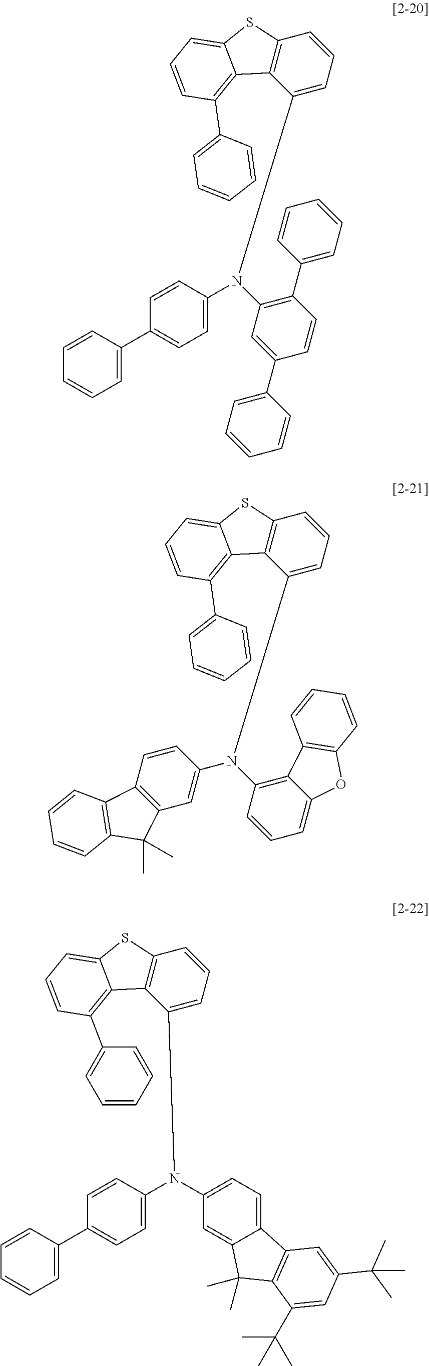

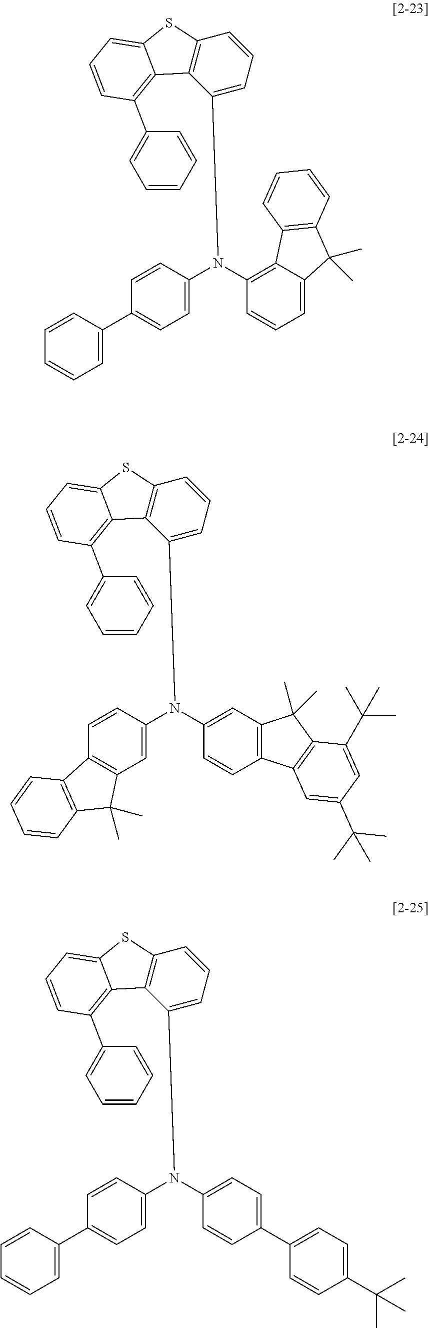 [SMILES: CC1(C)c2ccccc2-c2c(N(c3ccc(-c4ccccc4)cc3)c3cccc4sc5cccc(-c6ccccc6)c5c34)cccc21 ; CC(C)(C)c1cc2c(c(C(C)(C)C)c1)C(C)(C)c1cc(N(c3ccc4c(c3)C(C)(C)c3ccccc3-4)c3cccc4sc5cccc(-c6ccccc6)c5c34)ccc1-2 ; CC(C)(C)c1ccc(-c2ccc(N(c3ccc(-c4ccccc4)cc3)c3cccc4sc5cccc(-c6ccccc6)c5c34)cc2)cc1]

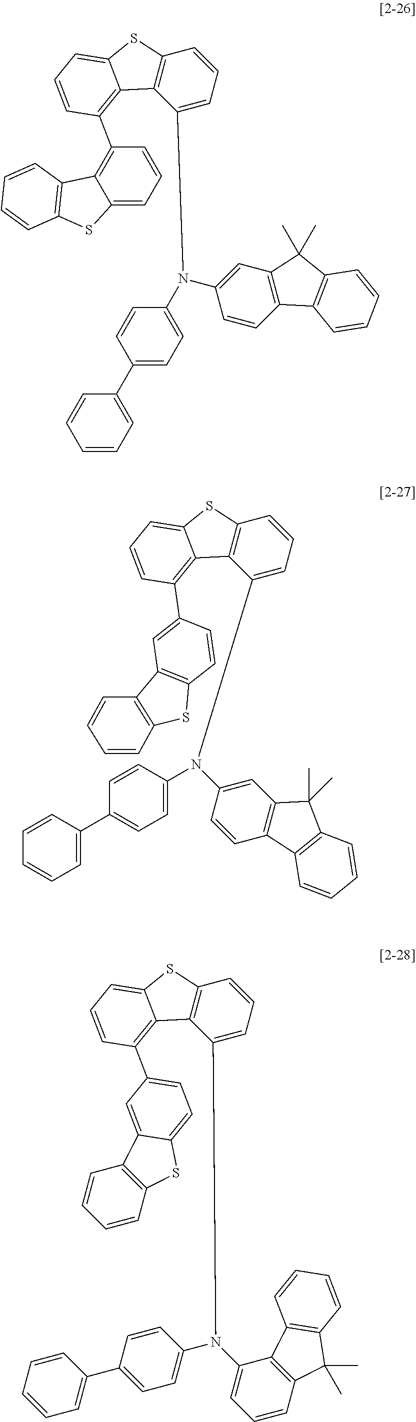 [SMILES: CC1(C)c2ccccc2-c2ccc(N(c3ccc(-c4ccccc4)cc3)c3cccc4sc5cccc(-c6cccc7sc8ccccc8c67)c5c34)cc21 ; CC1(C)c2ccccc2-c2ccc(N(c3ccc(-c4ccccc4)cc3)c3cccc4sc5cccc(-c6ccc7sc8ccccc8c7c6)c5c34)cc21 ; CC1(C)c2ccccc2-c2c(N(c3ccc(-c4ccccc4)cc3)c3cccc4sc5cccc(-c6ccc7sc8ccccc8c7c6)c5c34)cccc21]

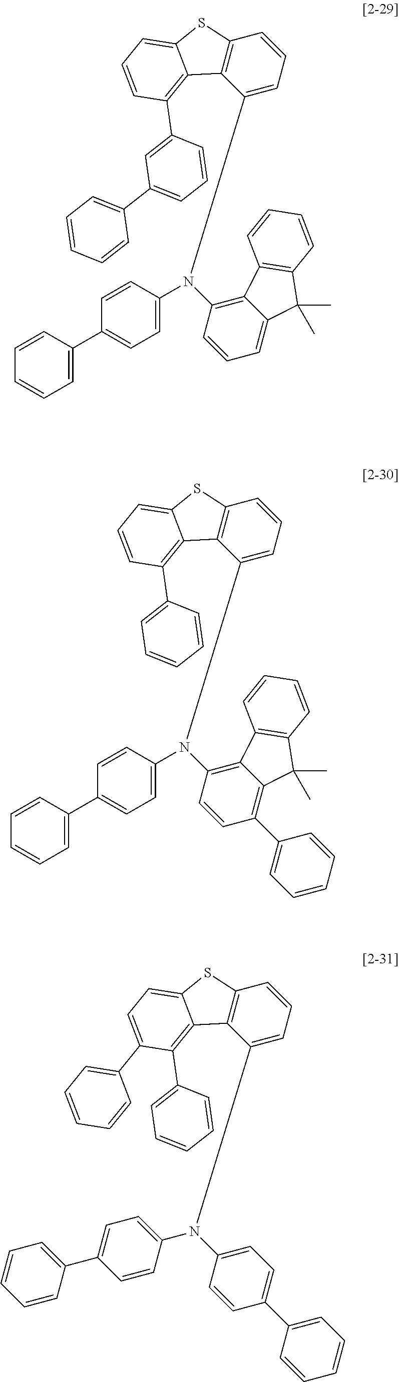 [SMILES: CC1(C)c2ccccc2-c2c(N(c3ccc(-c4ccccc4)cc3)c3cccc4sc5cccc(-c6cccc(-c7ccccc7)c6)c5c34)cccc21 ; CC1(C)c2ccccc2-c2c(N(c3ccc(-c4ccccc4)cc3)c3cccc4sc5cccc(-c6ccccc6)c5c34)ccc(-c3ccccc3)c21 ; c1ccc(-c2ccc(N(c3ccc(-c4ccccc4)cc3)c3cccc4sc5ccc(-c6ccccc6)c(-c6ccccc6)c5c34)cc2)cc1]

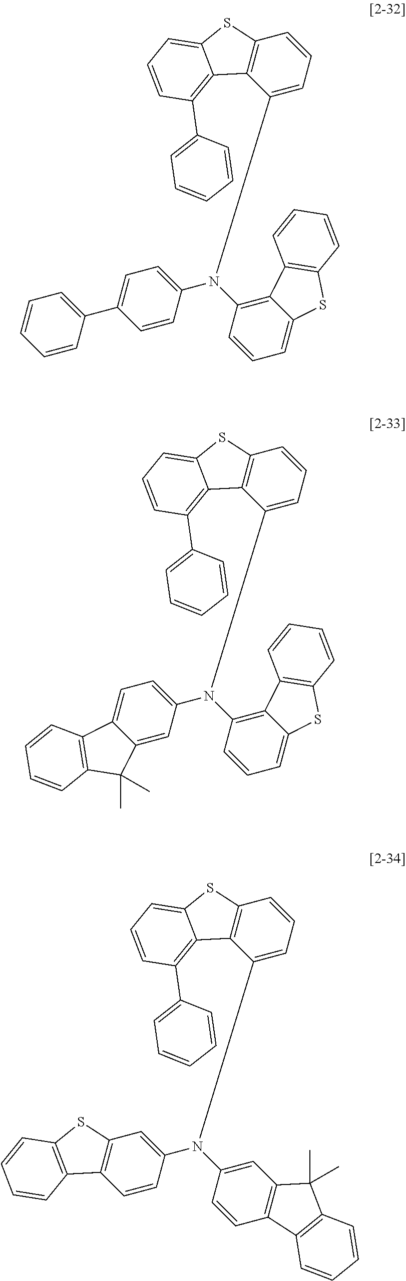 [SMILES: c1ccc(-c2ccc(N(c3cccc4sc5ccccc5c34)c3cccc4sc5cccc(-c6ccccc6)c5c34)cc2)cc1 ; CC1(C)c2ccccc2-c2ccc(N(c3cccc4sc5ccccc5c34)c3cccc4sc5cccc(-c6ccccc6)c5c34)cc21 ; CC1(C)c2ccccc2-c2ccc(N(c3ccc4c(c3)sc3ccccc34)c3cccc4sc5cccc(-c6ccccc6)c5c34)cc21]

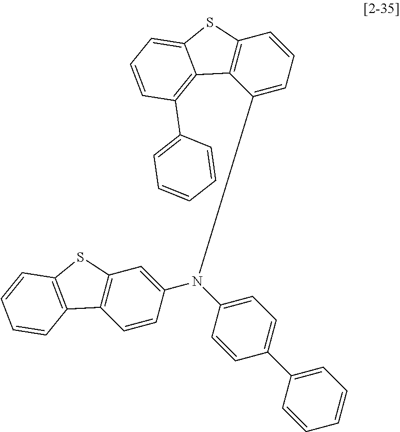 [SMILES: c1ccc(-c2ccc(N(c3ccc4c(c3)sc3ccccc34)c3cccc4sc5cccc(-c6ccccc6)c5c34)cc2)cc1]

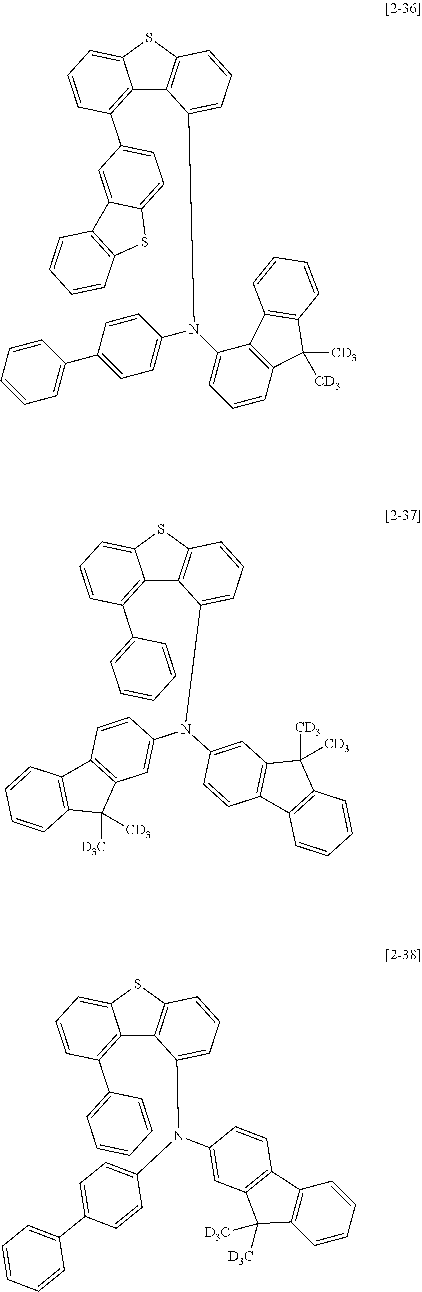 [SMILES: [2H]C([2H])([2H])C1(C([2H])([2H])[2H])c2ccccc2-c2c(N(c3ccc(-c4ccccc4)cc3)c3cccc4sc5cccc(-c6ccc7sc8ccccc8c7c6)c5c34)cccc21 ; [2H]C([2H])([2H])C1(C([2H])([2H])[2H])c2ccccc2-c2ccc(N(c3ccc4c(c3)C(C([2H])([2H])[2H])(C([2H])([2H])[2H])c3ccccc3-4)c3cccc4sc5cccc(-c6ccccc6)c5c34)cc21 ; [2H]C([2H])([2H])C1(C([2H])([2H])[2H])c2ccccc2-c2ccc(N(c3ccc(-c4ccccc4)cc3)c3cccc4sc5cccc(-c6ccccc6)c5c34)cc21]

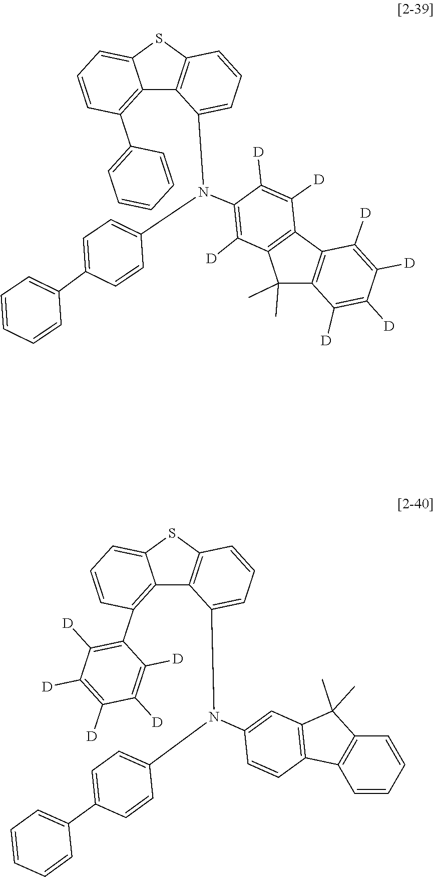 [SMILES: [2H]c1c([2H])c([2H])c2c(c1[2H])-c1c([2H])c([2H])c(N(c3ccc(-c4ccccc4)cc3)c3cccc4sc5cccc(-c6ccccc6)c5c34)c([2H])c1C2(C)C ; [2H]c1c([2H])c([2H])c(-c2cccc3sc4cccc(N(c5ccc(-c6ccccc6)cc5)c5ccc6c(c5)C(C)(C)c5ccccc5-6)c4c23)c([2H])c1[2H]]

In addition to the aforementioned compound for organic optoelectronic devices, one or more additional compounds may be included.

For example, a dopant may be further included.

The dopant may be, for example, a phosphorescent dopant, for example, a red, green or blue phosphorescent dopant, and may be, for example, a red or green phosphorescent dopant.

The dopant is a material mixed with the compound or composition for an organic optoelectronic device in a small amount to cause light emission, and may be generally a material such as a metal complex that emits light by multiple excitation into a triplet or more. The dopant may be, for example, an inorganic, organic, or organic-inorganic compound, and one or more types thereof may be used.

Examples of the dopant may be a phosphorescent dopant and examples of the phosphorescent dopant may be an organic metal compound including Ir, Pt, Os, Ti, Zr, Hf, Eu, Tb, Tm, Fe, Co, Ni, Ru, Rh, Pd, or a combination thereof. The phosphorescent dopant may be, for example, a compound represented by Chemical Formula Z, but is not limited thereto.

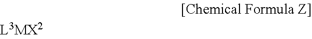

In Chemical Formula Z, M is a metal, and L³ and X² are the same or different, and are a ligand to form a complex compound with M.

The M may be for example Ir, Pt, Os, Ti, Zr, Hf, Eu, Tb, Tm, Fe, Co, Ni, Ru, Rh, Pd, or a combination thereof and the L³ and X² may be, for example a bidentate ligand.

Examples of ligands represented by L³ and X² may be selected from the chemical formulas listed in Group A, but are not limited thereto.

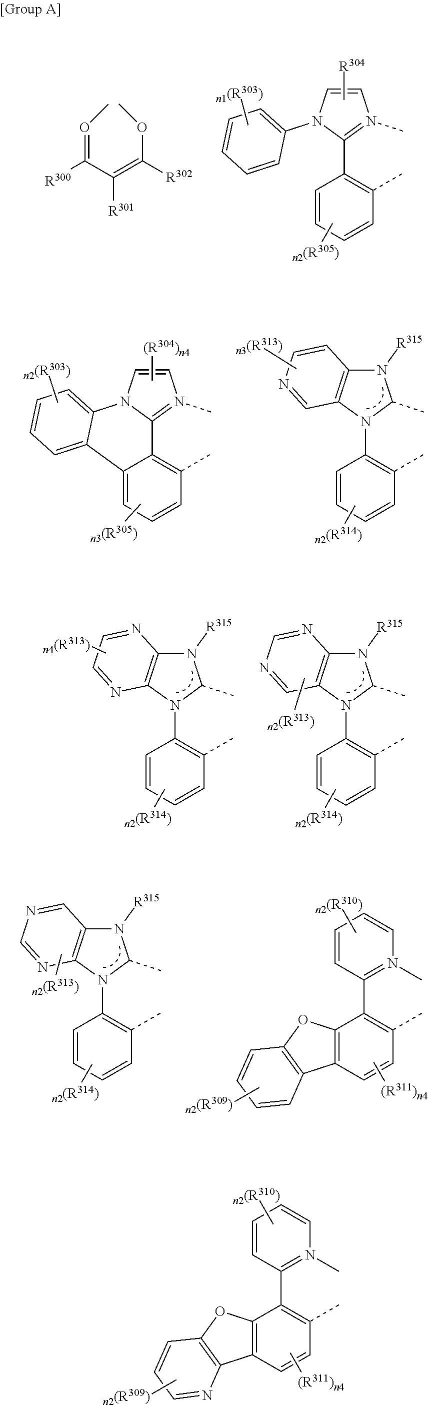

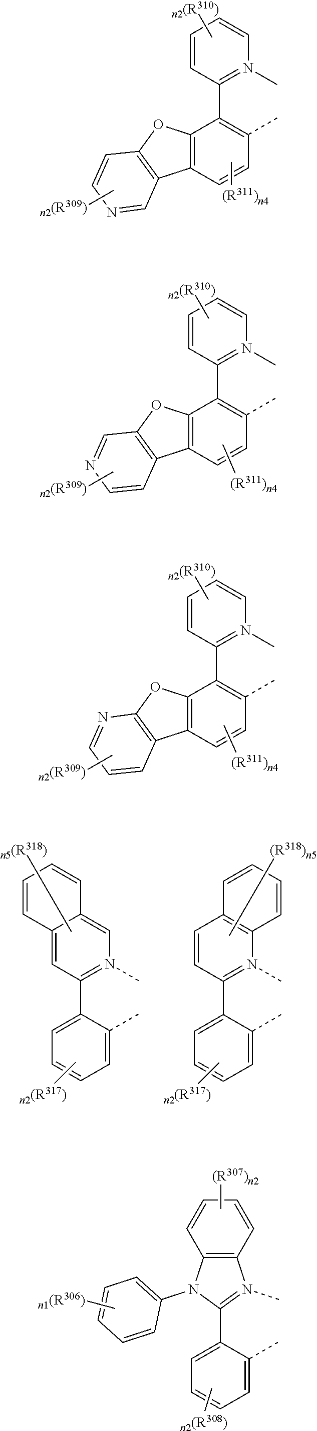

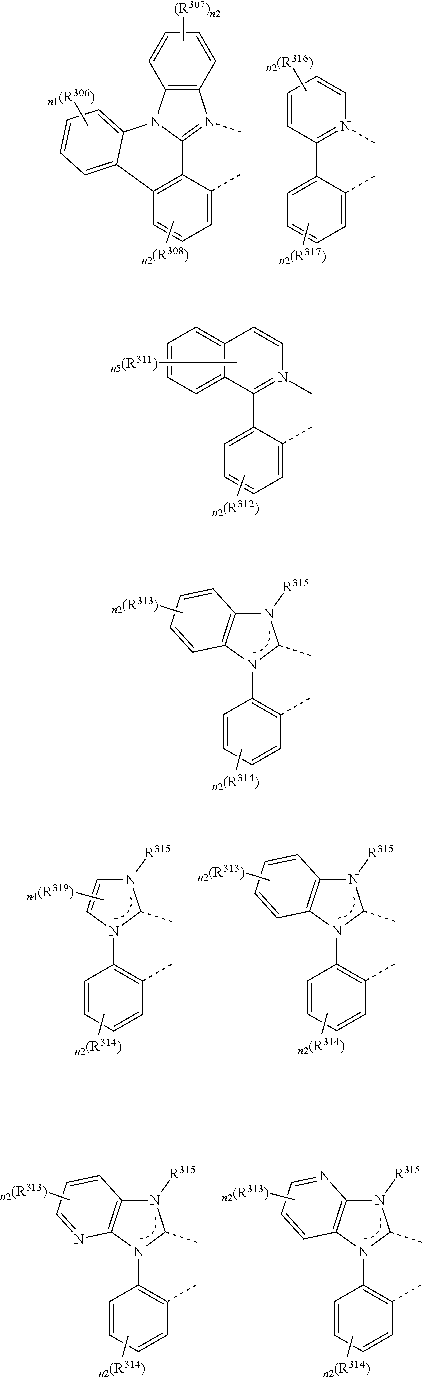

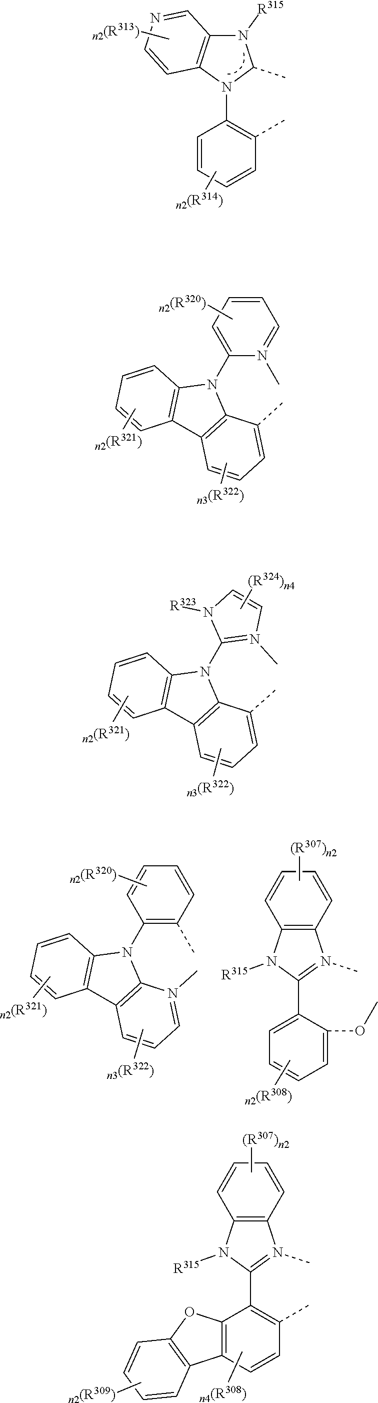

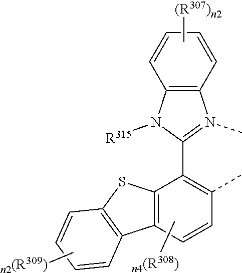

In Group A,
R³⁰⁰ to R³⁰² are each independently hydrogen, deuterium, a C1 to C30 alkyl group substituted or unsubstituted with a halogen, a C6 to C30 aryl group substituted or unsubstituted with a C1 to C30 alkyl or a halogen, and
R³⁰³ to R³²⁴ are each independently hydrogen, deuterium, a halogen, a substituted or unsubstituted C1 to C30 alkyl group, a substituted or unsubstituted C1 to C30 alkoxy group, a substituted or unsubstituted C3 to C30 cycloalkyl group, a substituted or unsubstituted C2 to C30 alkenyl group, a substituted or unsubstituted C6 to C30 aryl group, a substituted or unsubstituted C1 to C30 heteroaryl group, a substituted or unsubstituted C1 to C30 amino group, a substituted or unsubstituted C6 to C30 arylamino group, SF₅, a trialkylsilyl group having a substituted or unsubstituted C1 to C30 alkyl group, a dialkylarylsilyl group having a substituted or unsubstituted C1 to C30 alkyl group, and a C6 to C30 aryl group, or a triarylsilyl group having a substituted or unsubstituted C6 to C30 aryl group,
n1 is one of integers of 1 to 5,
n2 is one of integers of 1 to 4,
n3 is one of integers of 1 to 3,
n4 is an integer of 1 or 2, and
n5 is one of integers of 1 to 6.

When n1 is 2 or more, each substituent may be the same or different from each other.

When n2 is 2 or more, each substituent may be the same or different from each other.

When n3 is 2 or more, each substituent may be the same or different from each other.

When n4 is 2 or more, each substituent may be the same or different from each other.

When n5 is 2 or more, each substituent may be the same or different from each other.

The dopant according to an embodiment may be an iridium complex, for example, represented by Chemical Formula 4-1 or Chemical Formula 4-2.

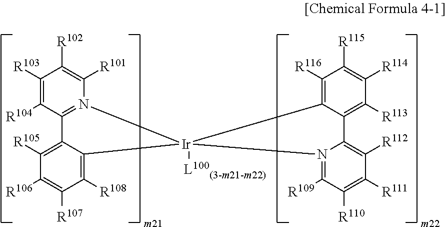

In Chemical Formula 4-1,
R¹⁰¹ to R¹¹⁶ are each independently hydrogen, deuterium, a substituted or unsubstituted C1 to C10 alkyl group, a substituted or unsubstituted C6 to C20 aryl group, or —SiR¹³²R¹³³R¹³⁴,
R¹³² to R¹³⁴ are each independently a substituted or unsubstituted C1 to C6 alkyl group,
at least one of R¹⁰¹ to R¹¹⁶ is a functional group represented by Chemical Formula V-1,
L¹⁰⁰ is a bidentate ligand of a monovalent anion, and is a ligand that coordinates to iridium through a lone pair of carbons or heteroatoms, and
m21 and m22 are each independently any one of integers of 0 to 3, and m21+m22 is any one of integers of 1 to 3,

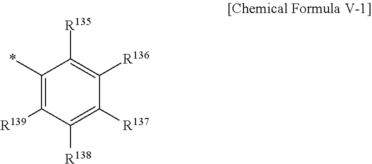

wherein, in Chemical Formula V-1,
R¹³⁵ to R¹³⁹ are each independently hydrogen, deuterium, a substituted or unsubstituted C1 to C10 alkyl group, a substituted or unsubstituted C6 to C20 aryl group, or —SiR¹³²R¹³³R¹³⁴, and
* means a portion linked to a carbon atom.

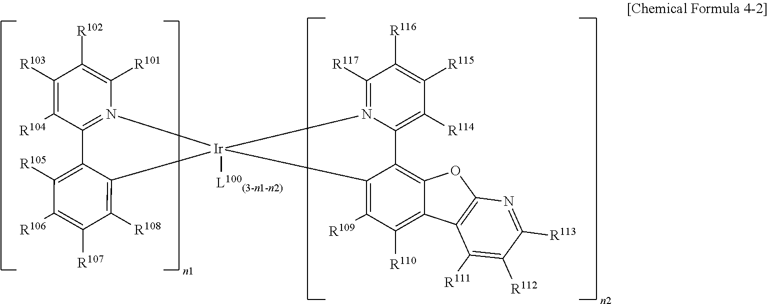

In Chemical Formula 4-2,
R¹⁰¹ to R¹¹⁷ are each independently hydrogen, deuterium, a substituted or unsubstituted C1 to C10 alkyl group, a substituted or unsubstituted C6 to C20 aryl group, or —SiR¹³³R¹³⁴R¹³⁵,
R¹³³ to R¹³⁵ are each independently a substituted or unsubstituted C1 to C6 alkyl group,
L¹⁰⁰ is a bidentate ligand of a monovalent anion, and is a ligand that coordinates to iridium through a lone pair of carbons or heteroatoms, and
n1 and n2 are independently any integers from 0 to 3, and n1+n2 are any one of integers of 1 to 3.

In another embodiment, the dopant may be a platinum complex, for example represented by Chemical Formula Z-1.

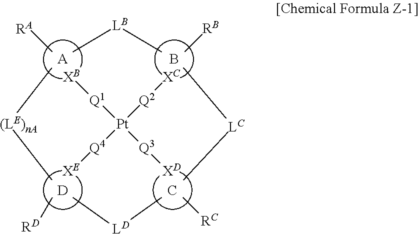

In Chemical Formula Z-1, rings A, B, C, and D are each independently a 5-membered or 6-membered carbocyclic or heterocyclic ring;
R^{A}, R^{B}, R^{C}, and R^{D} are each independently mono-, di-, tri-, or tetra-substitution, or unsubstitution;
L^{B}, L^{C}, and L^{D} are each independently a direct bond, BR, NR, PR, O, S, Se, C═O, S═O. SO₂, CRR′, SiRR′, GeRR′, or a combination thereof,
when nA is 1, L^{E} may be a direct bond, BR, NR, PR, O, S, Se, C═O, S═O, SO₂, CRR′, SiRR′, GeRR′, or a combination thereof, and when nA is 0, L^{E} does not exist;
R^{A}, R^{B}, R^{C}, R^{D}, R, and R′ are each independently hydrogen, deuterium, a halogen, alkyl group, a cycloalkyl group, a heteroalkyl group, an arylalkyl group, an alkoxy group, an aryloxy group, an amino group, a silyl group, an alkenyl group, a cycloalkenyl group, a heteroalkenyl group, an alkynyl group, an aryl group, a heteroaryl group, an acyl group, a carbonyl group, a carboxylic acid group, an ester group, a nitrile group, an isonitrile group, a sulfanyl group, a sulfinyl group, a sulfonyl group, a phosphino group, or a combination thereof, any adjacent R^{A}R^{B}, R^{C}, R^{D}, R, and R′ are optionally linked to each other to provide a ring; X^{B}, X^{C}, X^{D}, and X^{E} are each independently selected from carbon and nitrogen; and Q¹, Q², Q³, and Q⁴ each represent oxygen or a direct bond.

The platinum a complex may be for example represented by Chemical Formula 5-1 or Chemical Formula 5-2.

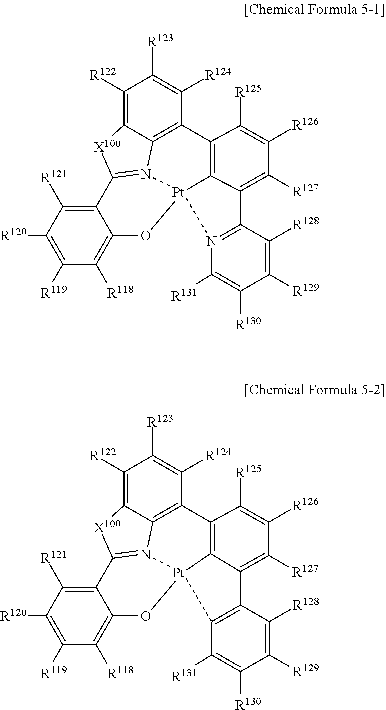

In Chemical Formula 5-1 and Chemical Formula 5-2,
X¹⁰⁰ is selected from O, S, and NR¹³²
R¹¹⁸ to R¹³² are each independently hydrogen, deuterium, a substituted or unsubstituted C1 to C10 alkyl group, a substituted or unsubstituted C6 to C20 aryl group, or —SiR¹³³R¹³⁴R¹³⁵,
R¹³³ to R¹³⁵ are each independently a substituted or unsubstituted C1 to C6 alkyl group, at least one of R¹¹⁸ to R¹³² is —SiR¹³³R¹³⁴R¹³⁵ or a tert-butyl group, and
R¹³³ to R¹³⁵ are each independently a substituted or unsubstituted C1 to C6 alkyl group.

An organic optoelectronic device using the aforementioned compound for an organic optoelectronic device is described.

The organic optoelectronic device may be a suitable device to convert electrical energy into photoenergy and vice versa, e.g., an organic photoelectric device, an organic light emitting diode, an organic solar cell, or an organic photoconductor drum.

Herein, an organic light emitting diode as one example of an organic optoelectronic device is described referring to drawings.

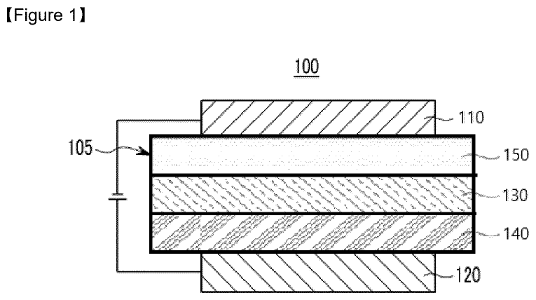

FIG. 1 is a cross-sectional view showing an organic light emitting diode according to an embodiment.

Referring to FIG. 1, an organic light emitting diode 100 according to an embodiment includes an anode 120 and a cathode 110 facing each other and an organic layer 105 disposed between the anode 120 and cathode 110.

The anode 120 may be made of a conductor having a large work function to help hole injection, and may be for example a metal, a metal oxide and/or a conductive polymer. The anode 120 may be, for example a metal such as nickel, platinum, vanadium, chromium, copper, zinc, gold, and the like or an alloy thereof; a metal oxide such as zinc oxide, indium oxide, indium tin oxide (ITO), indium zinc oxide (IZO), and the like; a combination of a metal and an oxide such as ZnO and Al or SnO₂ and Sb; a conductive polymer such as poly(3-methylthiophene), poly(3,4-(ethylene-1,2-dioxy)thiophene) (PEDOT), polypyrrole, and polyaniline, but is not limited thereto.

The cathode 110 may be made of a conductor having a small work function to help electron injection, and may be for example a metal, a metal oxide, and/or a conductive polymer.

The cathode 110 may be for example a metal such as magnesium, calcium, sodium, potassium, titanium, indium, yttrium, lithium, gadolinium, aluminum silver, tin, lead, cesium, barium, and the like, or an alloy thereof; a multi-layer structure material such as LiF/Al, LiO₂/Al, LiF/Ca, LiF/Al, and BaF₂/Ca, but is not limited thereto.

The organic layer 105 may include the aforementioned compound for the organic optoelectronic device.

The organic layer 105 may include a light emitting layer 130, and the light emitting layer 130 may include the aforementioned compound for the organic optoelectronic device.

The composition for the organic optoelectronic device further including a dopant may be, for example, a green light emitting composition.

The light emitting layer 130 may include, for example, the aforementioned compound for an organic optoelectronic device as a phosphorescent host.

The organic layer may further include a charge transport region in addition to the light emitting layer.

The charge transport region may be, for example, a hole transport region 140.

The hole transport region 140 can further increase hole injection and/or hole mobility and block electrons between the anode 120 and the light emitting layer 130.

Specifically, the hole transport region 140 may include a hole transport layer between the anode 120 and the light emitting layer 130, and a hole transport auxiliary layer between the light emitting layer 130 and the hole transport layer, and, and the hole transport auxiliary layer may include the aforementioned compound for the organic optoelectronic device.

At this time, the light emitting layer includes a host and a dopant, and the host may be, for example, a phosphorescent host.

The phosphorescent host should facilitate injection and transport of holes and electrons within the light emitting layer, ultimately holes and electrons should meet to form exciton well, and the formed exciton energy should be well transferred to the dopant. Examples of phosphorescent hosts may include an organic compound including carbazole, indolocarbazole, dibenzofuran, dibenzothiophene, indolodibenzofuran, indolodibenzothiophene, fluorene, indenocarbazole, triphenylene, pyrimidine, triazine, or a combination thereof.

The phosphorescent host may be used without limitation as long as it is a known material and may be, for example, a single host or a mixed host.

In addition, the aforementioned compound for an organic optoelectronic device may be included in the light emitting layer, and at least one of the compounds listed in Group C may be included in at least one layer of the hole transport layer and the hole transport auxiliary layer.

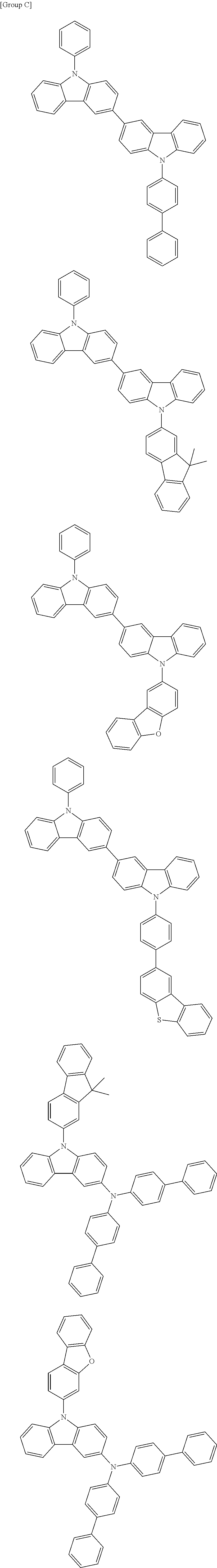

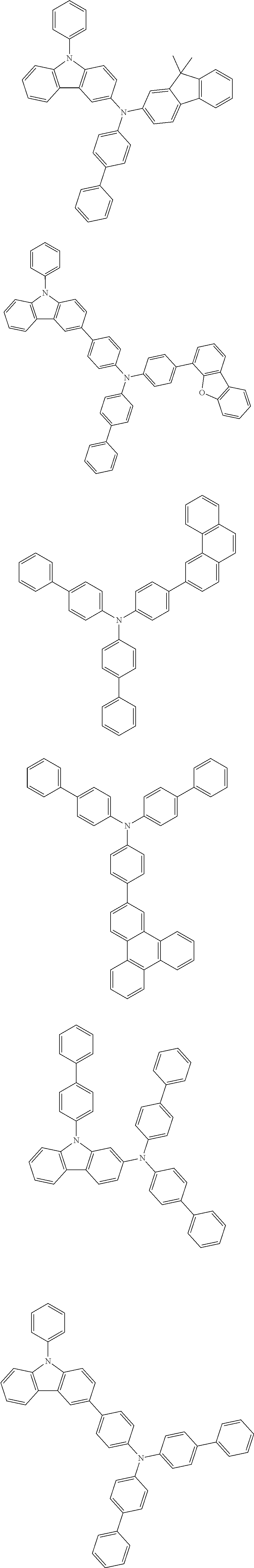

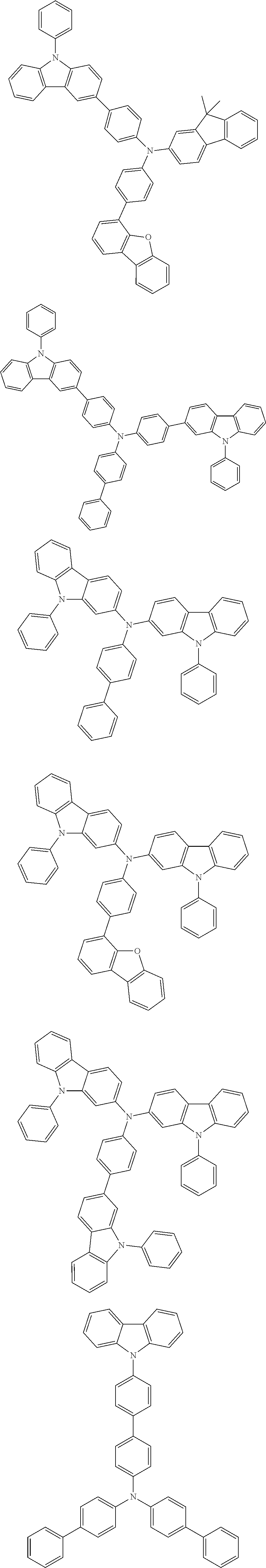

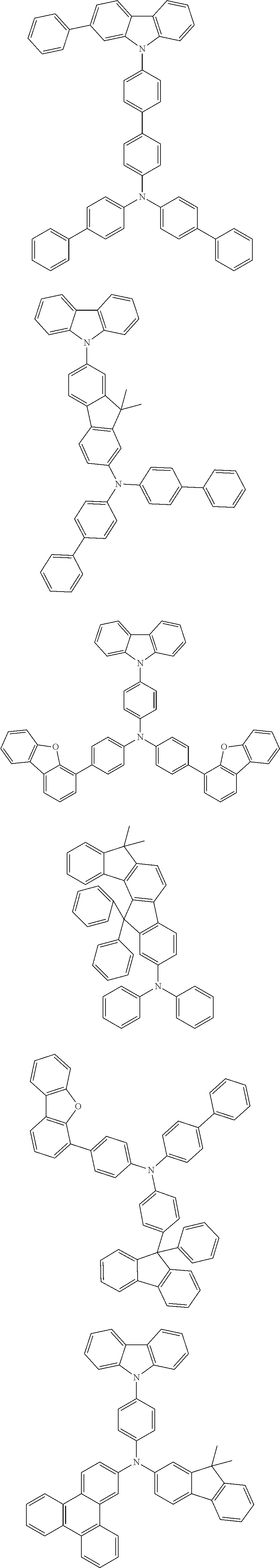

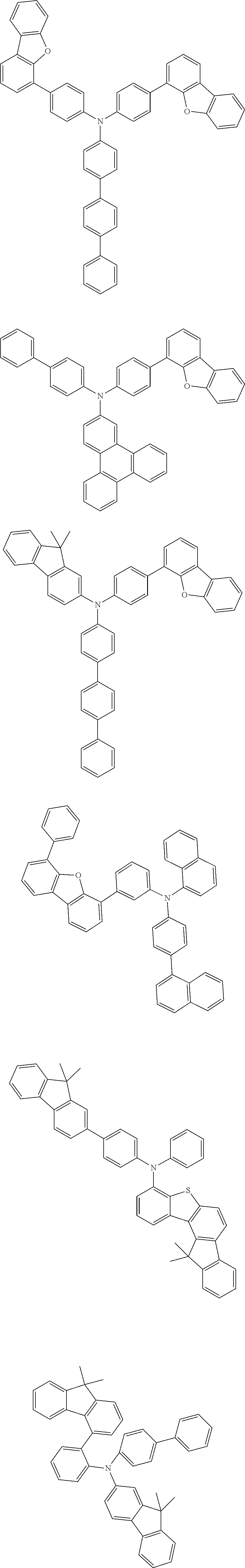

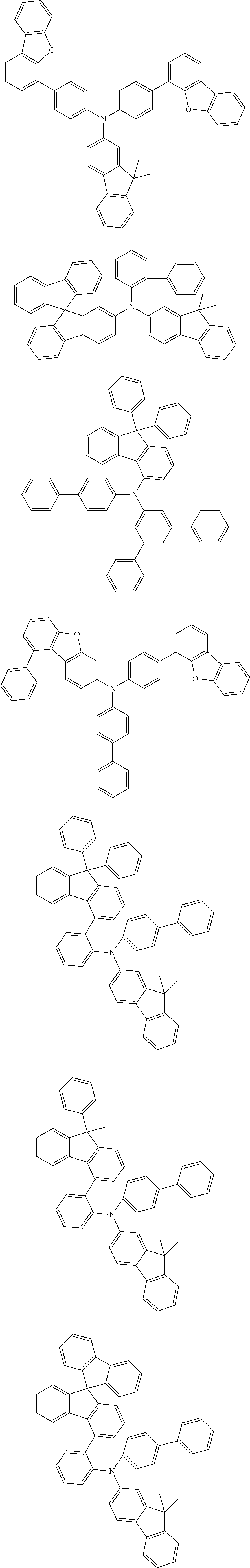

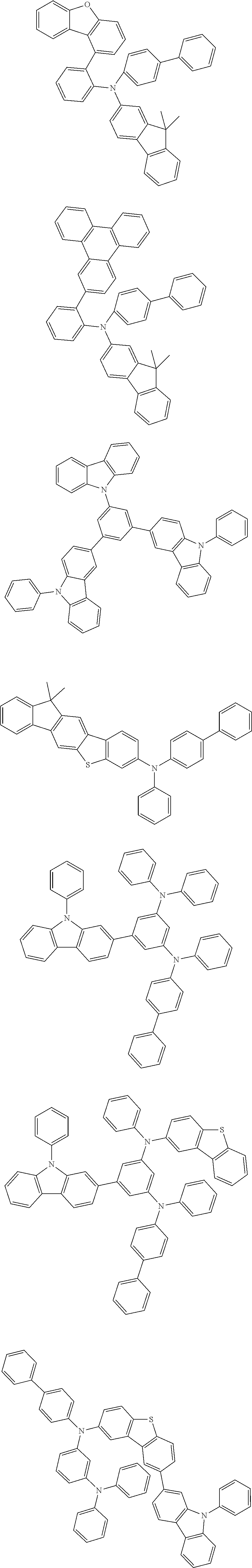

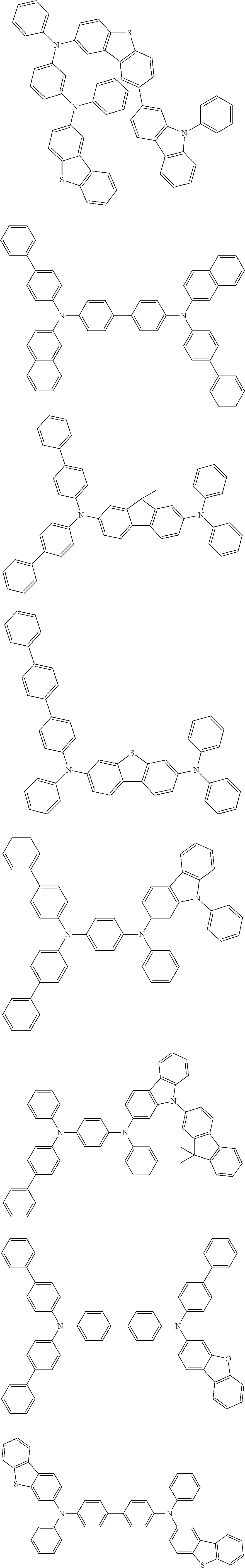

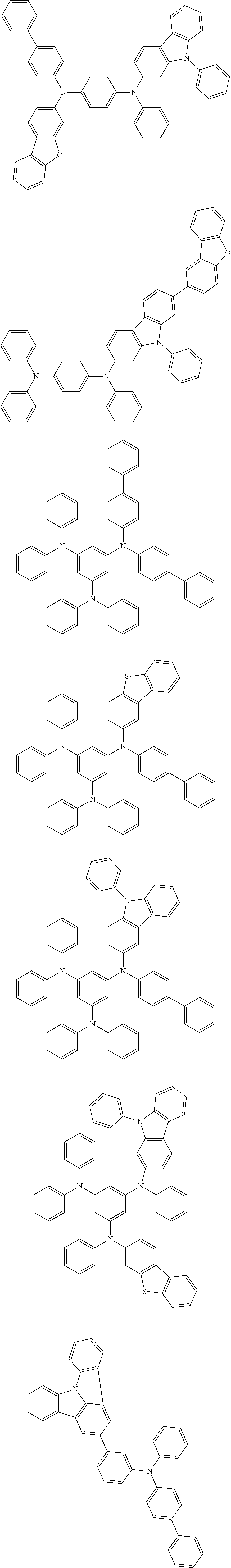

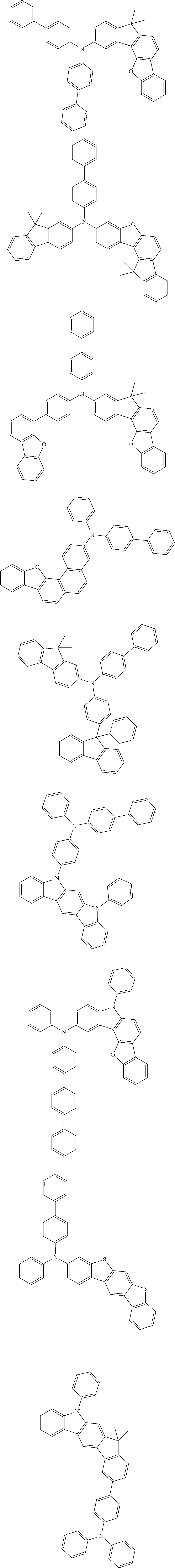

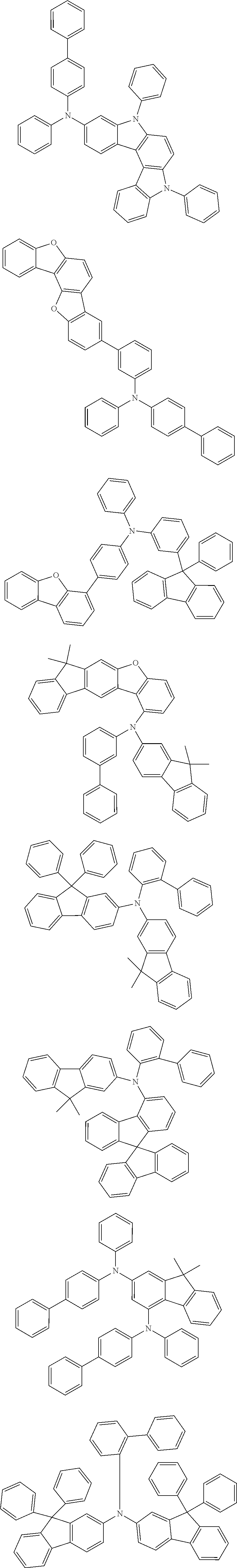

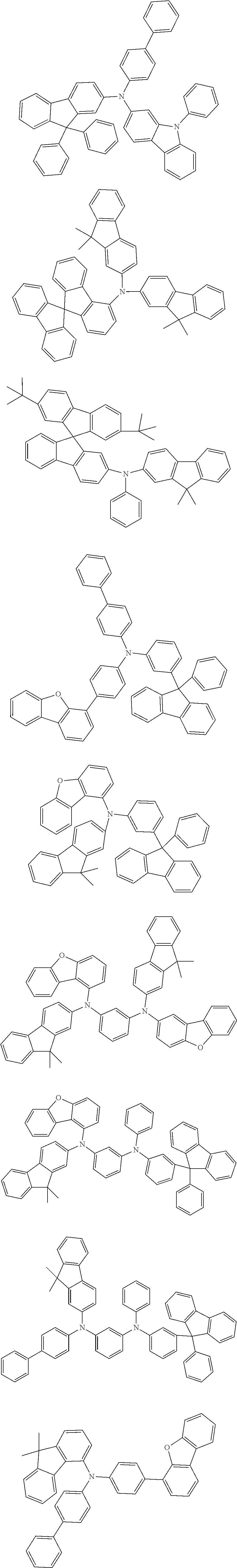

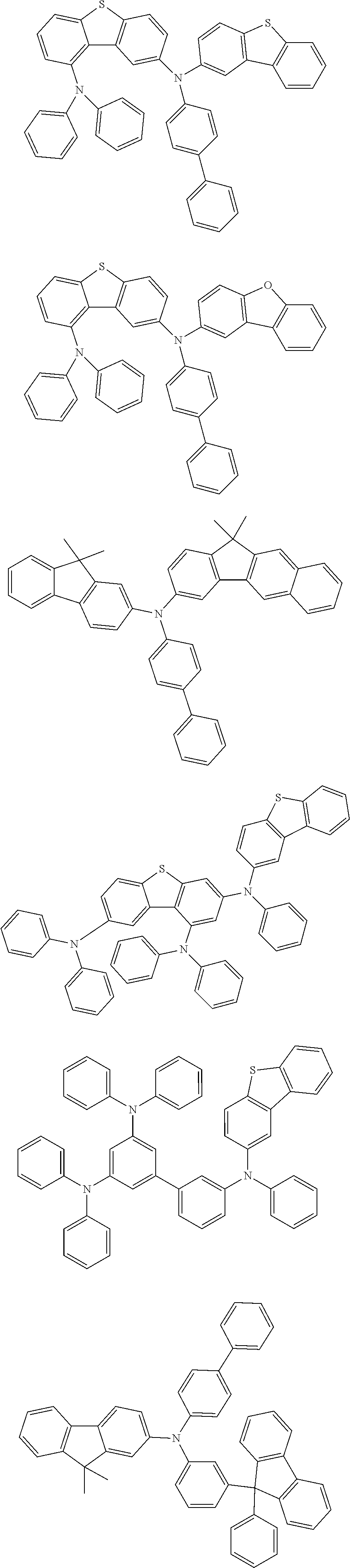

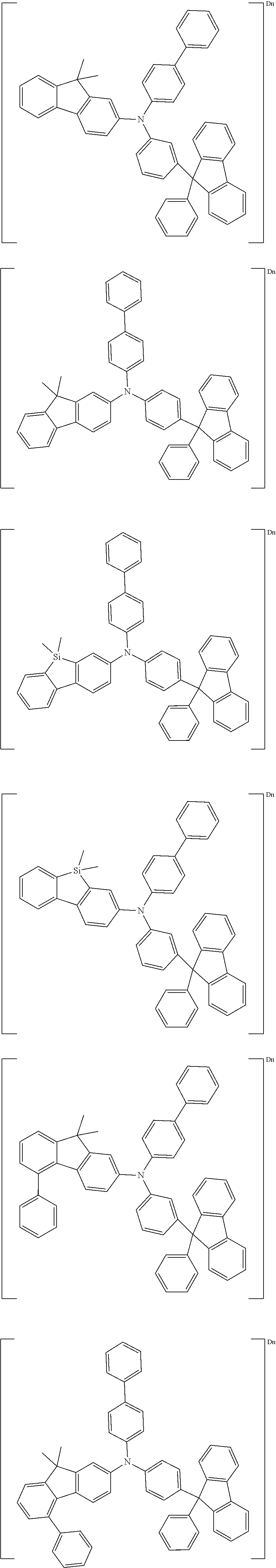

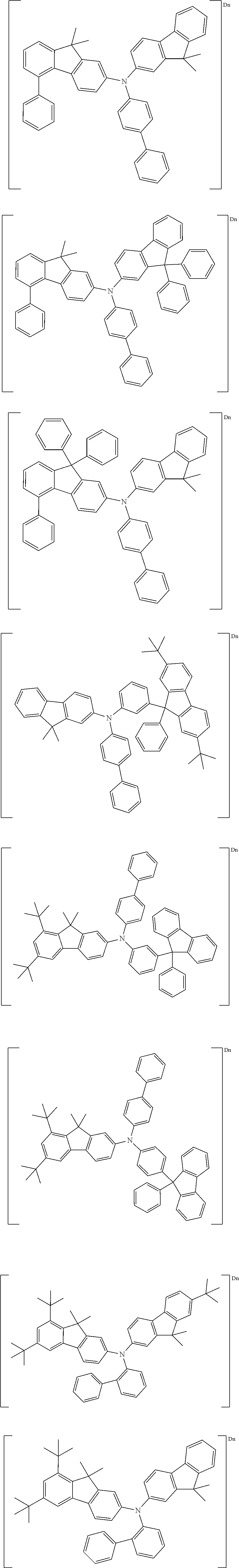

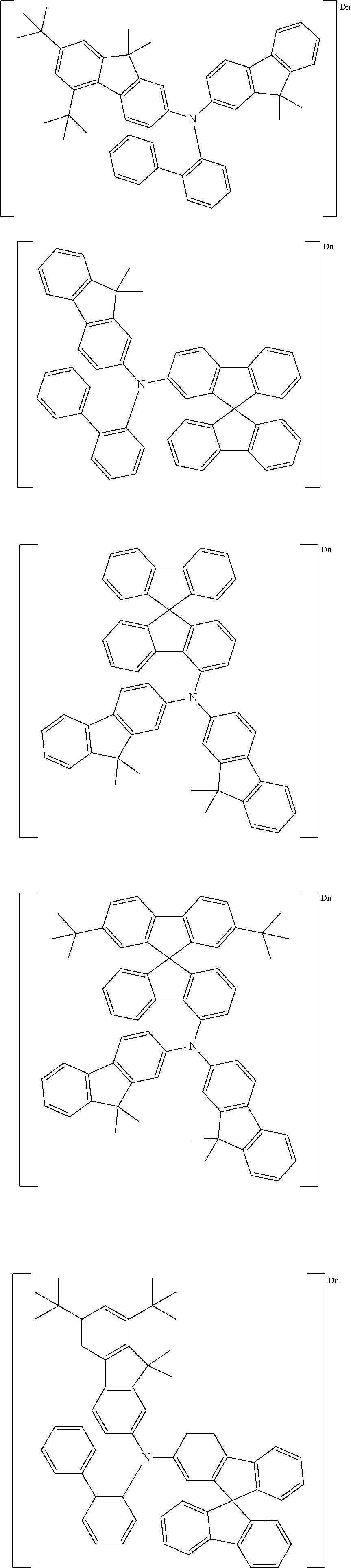

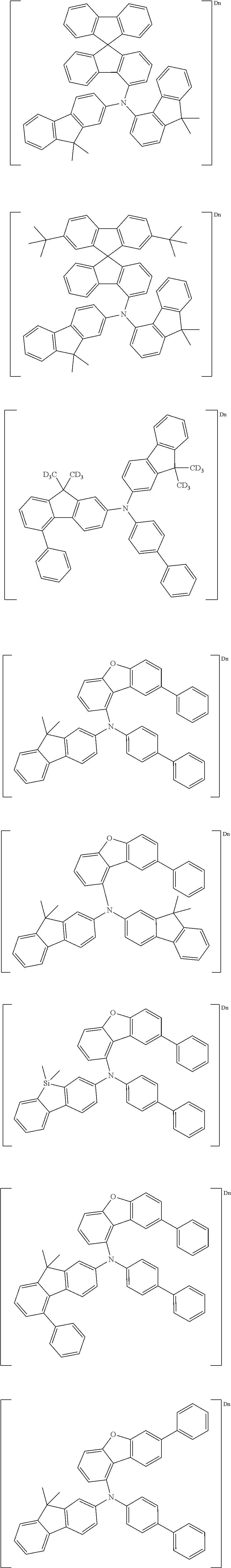

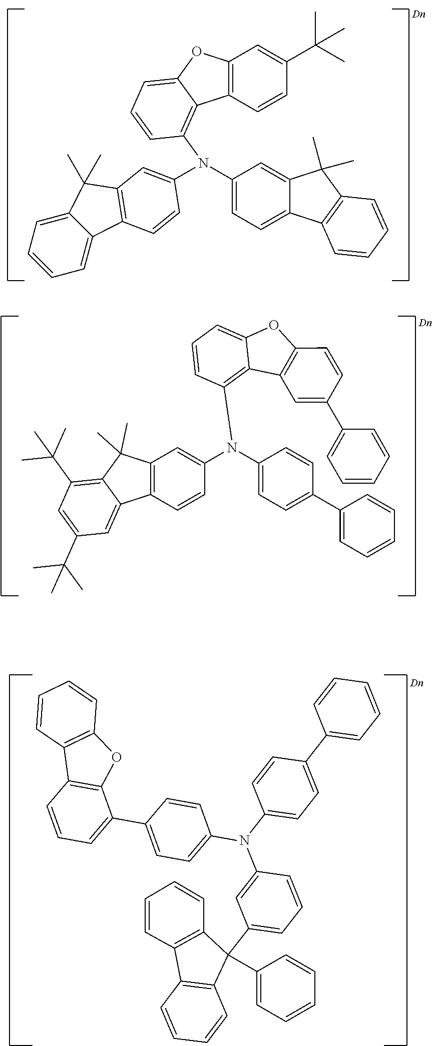

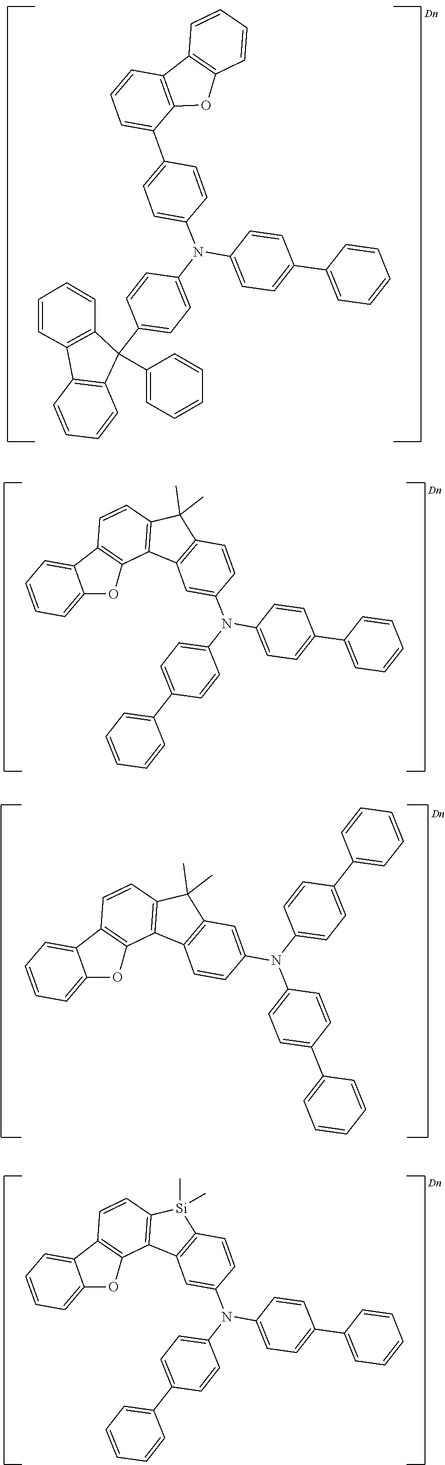

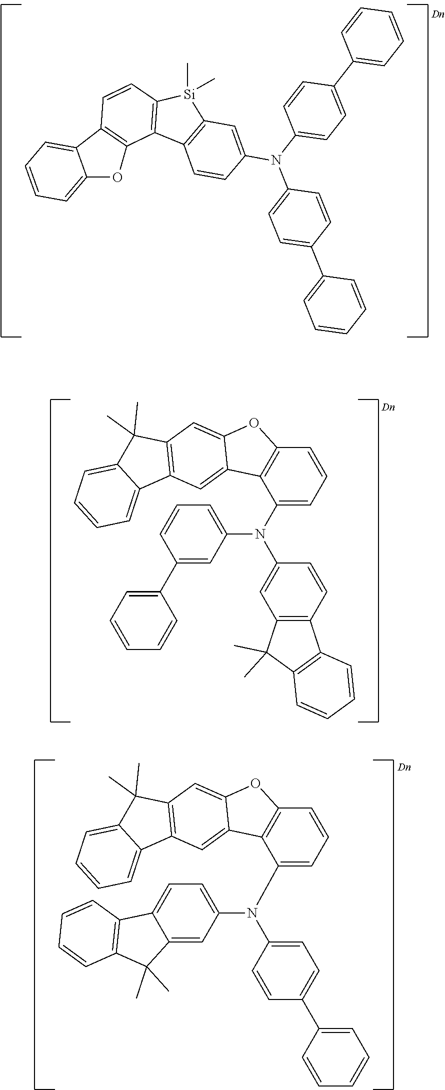

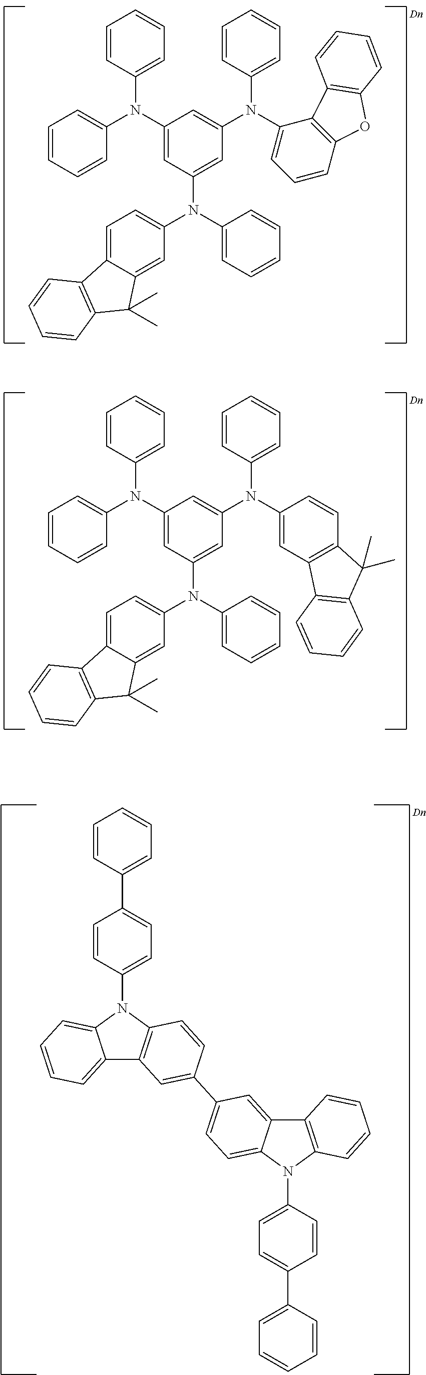

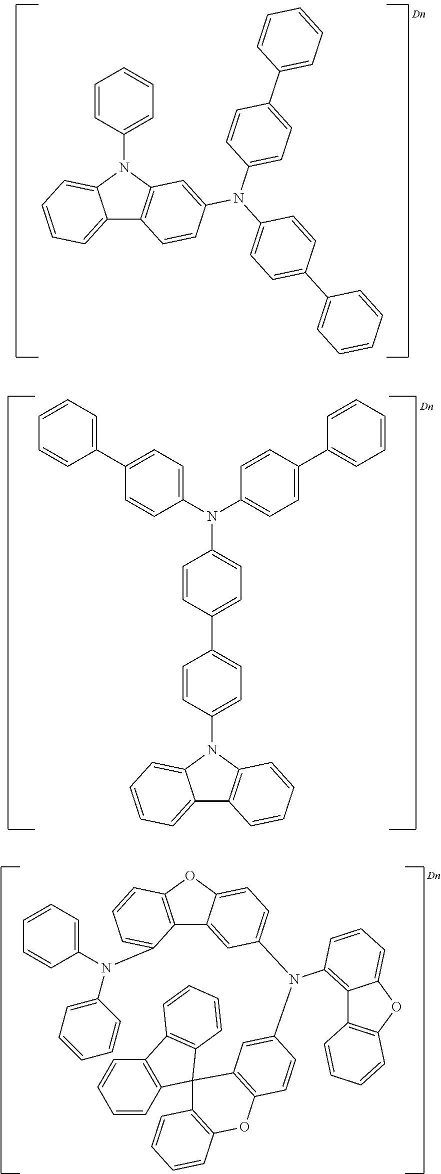

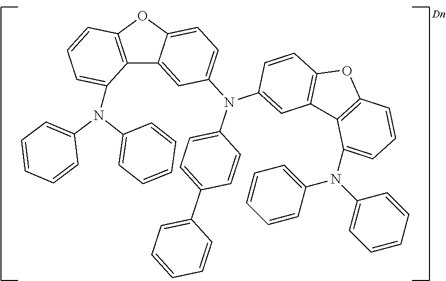

(Dn Refers to the Number of Hydrogens Replaced with Deuterium and Indicates a Structure Substituted with One or More Deuteriums)

In the hole transport region, in addition to the compounds described above, known compounds disclosed in U.S. Pat. No. 5,061,569A, JP1993-009471A, WO1995-009147A1, JP1995-126615A, JP1998-095973A, etc. and compounds having a similar structure may also be used.

Also, the charge transport region may be, for example, the electron transport region 150.

The electron transport region 150 may further increase electron injection and/or electron mobility and block holes between the cathode 110 and the light emitting layer 130.

Specifically, the electron transport region 150 may include an electron transport layer between the cathode 110 and the light emitting layer 130, and an electron transport auxiliary layer between the light emitting layer 130 and the electron transport layer, and at least one of the compounds of Group D may be included in at least one of the electron transport layer and the electron transport auxiliary layer.

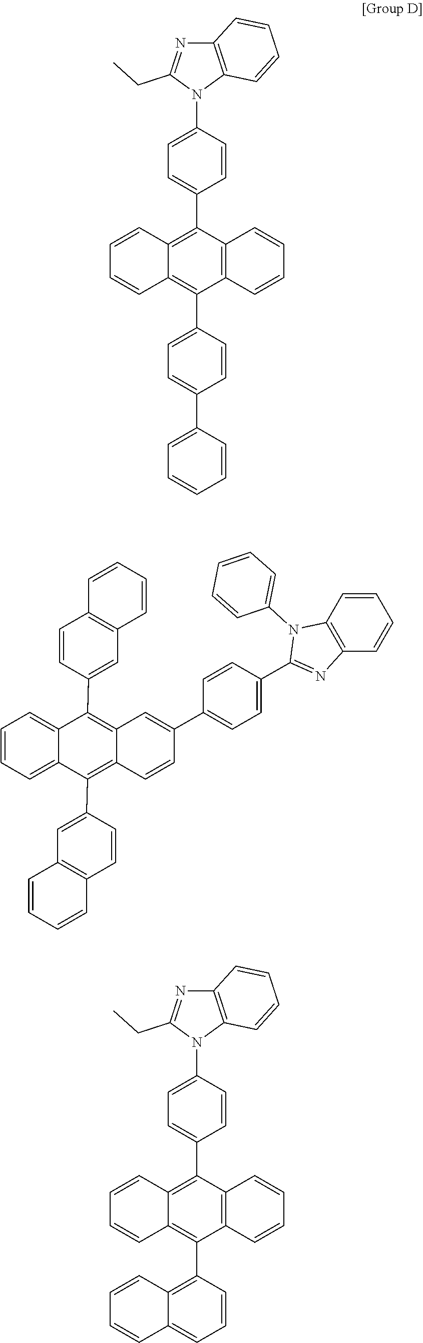 [SMILES: CCc1nc2ccccc2n1-c1ccc(-c2c3ccccc3c(-c3ccc(-c4ccccc4)cc3)c3ccccc23)cc1 ; c1ccc(-n2c(-c3ccc(-c4ccc5c(-c6ccc7ccccc7c6)c6ccccc6c(-c6ccc7ccccc7c6)c5c4)cc3)nc3ccccc32)cc1 ; CCc1nc2ccccc2n1-c1ccc(-c2c3ccccc3c(-c3cccc4ccccc34)c3ccccc23)cc1]

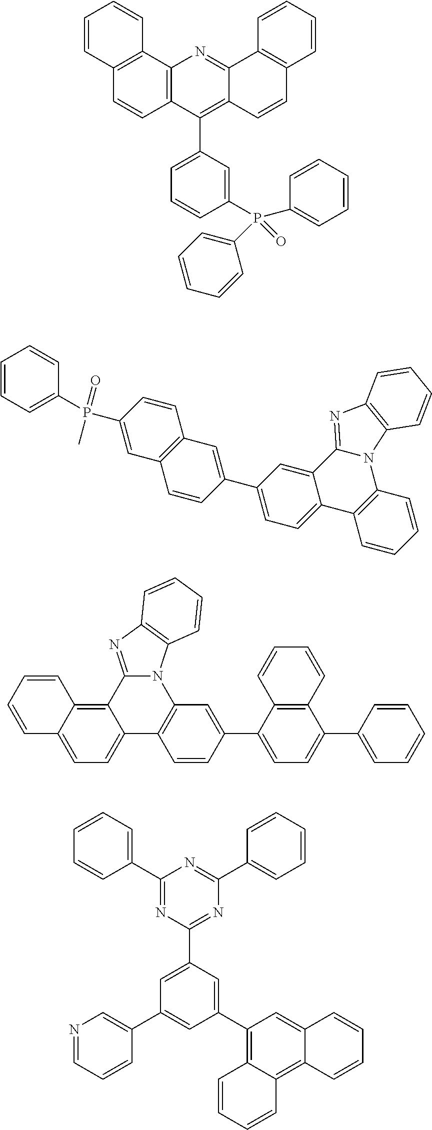 [SMILES: O=P(c1ccccc1)(c1ccccc1)c1cccc(-c2c3ccc4ccccc4c3nc3c2ccc2ccccc23)c1 ; CP(=O)(c1ccccc1)c1ccc2cc(-c3ccc4c5ccccc5n5c6ccccc6nc5c4c3)ccc2c1 ; c1ccc(-c2ccc(-c3ccc4c5ccc6ccccc6c5c5nc6ccccc6n5c4c3)c3ccccc23)cc1 ; c1ccc(-c2nc(-c3ccccc3)nc(-c3cc(-c4cccnc4)cc(-c4cc5ccccc5c5ccccc45)c3)n2)cc1]

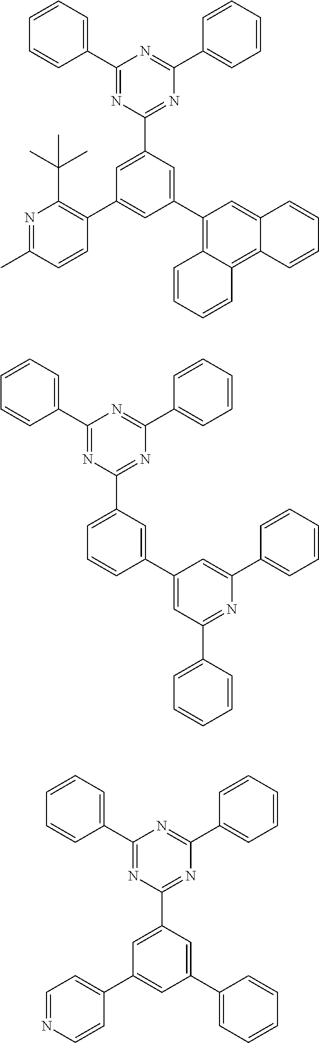

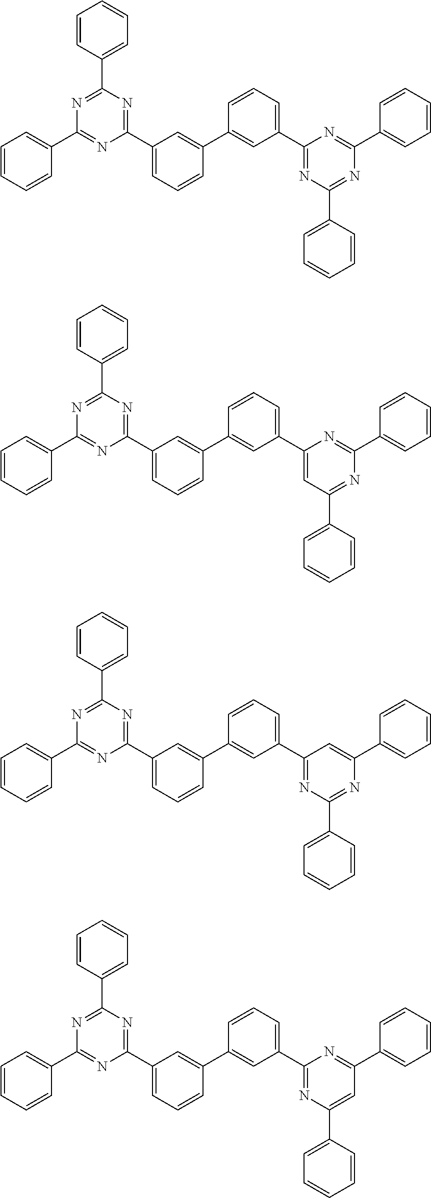 [SMILES: c1ccc(-c2nc(-c3ccccc3)nc(-c3cccc(-c4cccc(-c5nc(-c6ccccc6)nc(-c6ccccc6)n5)c4)c3)n2)cc1 ; c1ccc(-c2cc(-c3cccc(-c4cccc(-c5nc(-c6ccccc6)nc(-c6ccccc6)n5)c4)c3)nc(-c3ccccc3)n2)cc1 ; c1ccc(-c2cc(-c3cccc(-c4cccc(-c5nc(-c6ccccc6)nc(-c6ccccc6)n5)c4)c3)nc(-c3ccccc3)n2)cc1 ; c1ccc(-c2cc(-c3ccccc3)nc(-c3cccc(-c4cccc(-c5nc(-c6ccccc6)nc(-c6ccccc6)n5)c4)c3)n2)cc1]

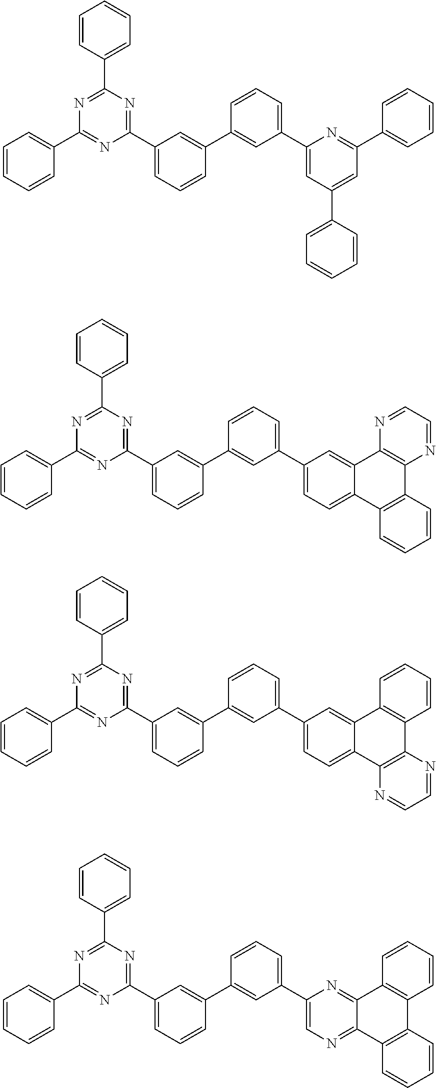 [SMILES: c1ccc(-c2cc(-c3ccccc3)nc(-c3cccc(-c4cccc(-c5nc(-c6ccccc6)nc(-c6ccccc6)n5)c4)c3)c2)cc1 ; c1ccc(-c2nc(-c3ccccc3)nc(-c3cccc(-c4cccc(-c5ccc6c7ccccc7c7nccnc7c6c5)c4)c3)n2)cc1 ; c1ccc(-c2nc(-c3ccccc3)nc(-c3cccc(-c4cccc(-c5ccc6c(c5)c5ccccc5c5nccnc65)c4)c3)n2)cc1 ; c1ccc(-c2nc(-c3ccccc3)nc(-c3cccc(-c4cccc(-c5cnc6c7ccccc7c7ccccc7c6n5)c4)c3)n2)cc1]

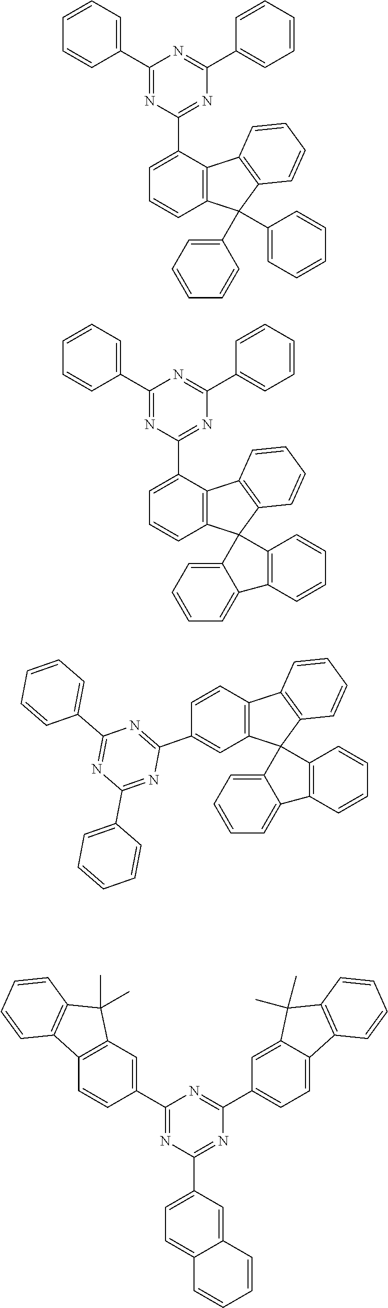

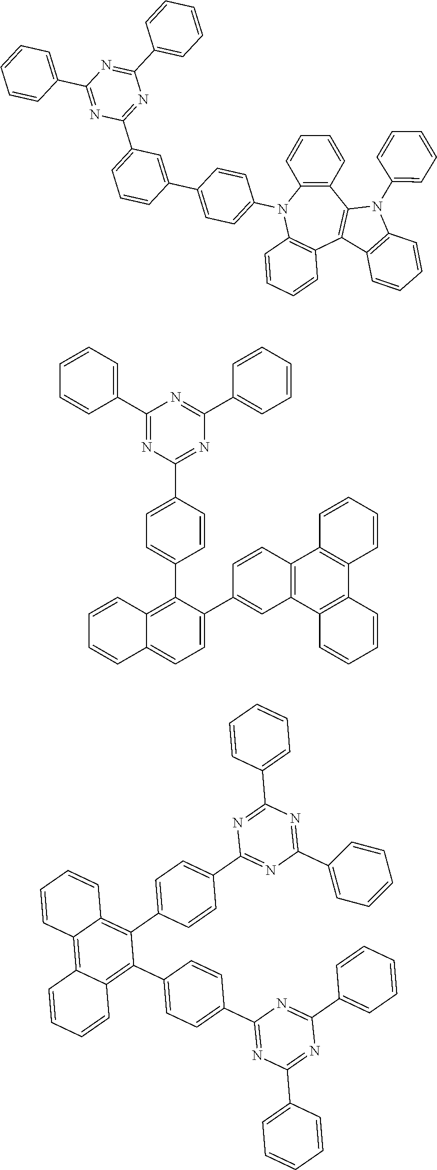 [SMILES: c1ccc(-c2nc(-c3ccccc3)nc(-c3cccc(-c4ccc(N5c6ccccc6-c6c(n(-c7ccccc7)c7ccccc67)-c6ccccc65)cc4)c3)n2)cc1 ; c1ccc(-c2nc(-c3ccccc3)nc(-c3ccc(-c4c(-c5ccc6c7ccccc7c7ccccc7c6c5)ccc5ccccc45)cc3)n2)cc1 ; c1ccc(-c2nc(-c3ccccc3)nc(-c3ccc(-c4c(-c5ccc(-c6nc(-c7ccccc7)nc(-c7ccccc7)n6)cc5)c5ccccc5c5ccccc45)cc3)n2)cc1]

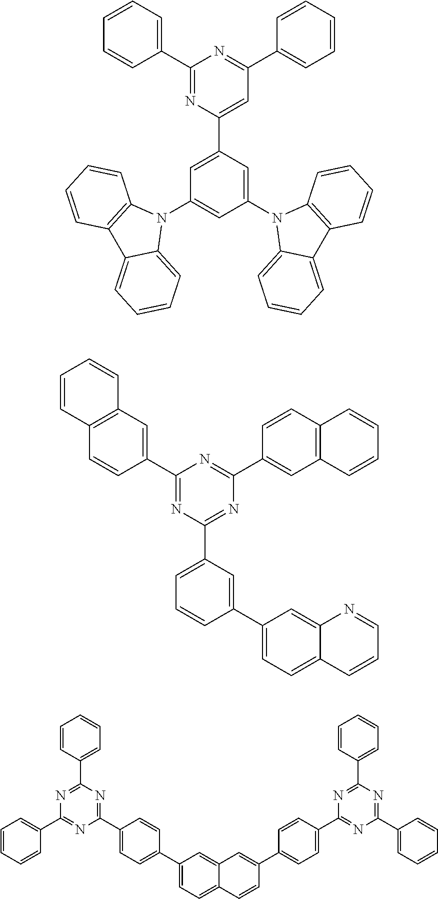

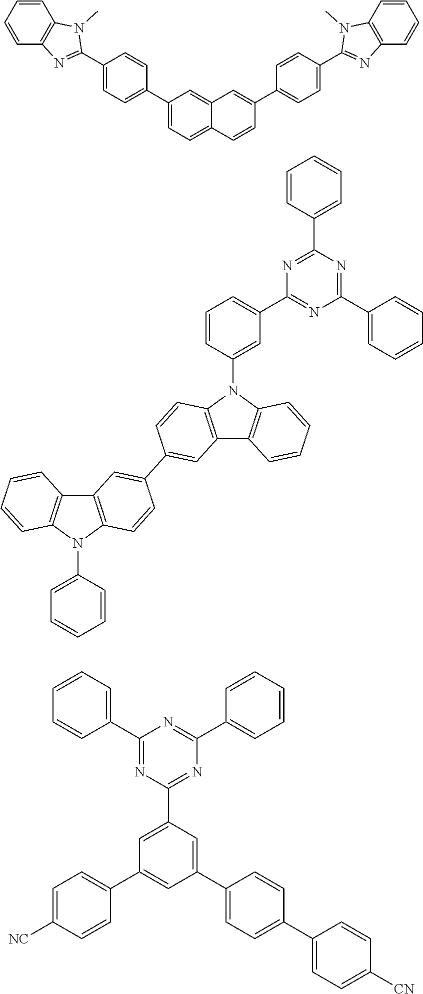

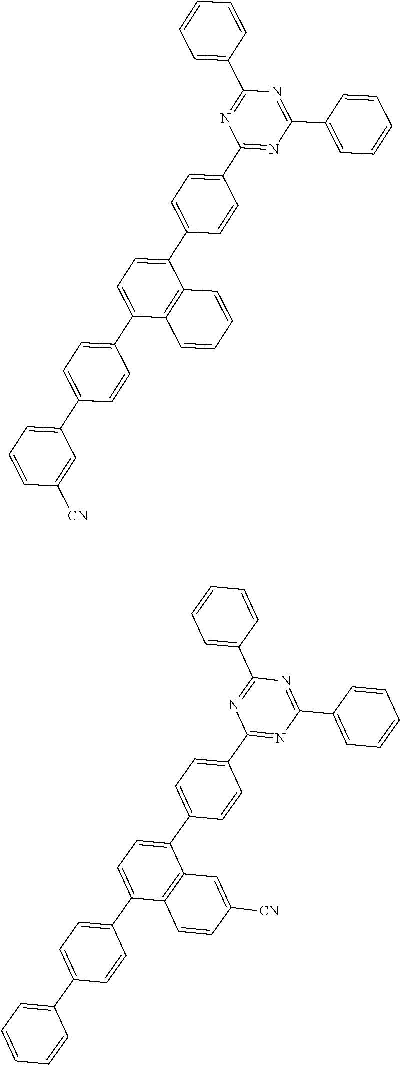 [SMILES: N#Cc1cccc(-c2ccc(-c3ccc(-c4ccc(-c5nc(-c6ccccc6)nc(-c6ccccc6)n5)cc4)c4ccccc34)cc2)c1 ; N#Cc1ccc2c(-c3ccc(-c4ccccc4)cc3)ccc(-c3ccc(-c4nc(-c5ccccc5)nc(-c5ccccc5)n4)cc3)c2c1]

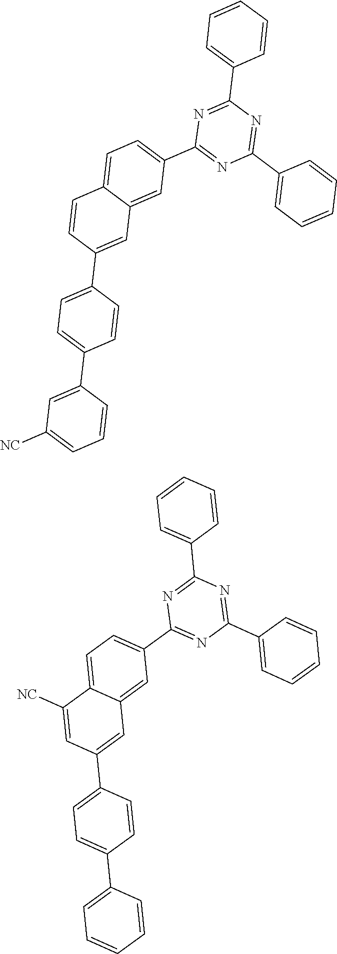 [SMILES: N#Cc1cccc(-c2ccc(-c3ccc4ccc(-c5nc(-c6ccccc6)nc(-c6ccccc6)n5)cc4c3)cc2)c1 ; N#Cc1cc(-c2ccc(-c3ccccc3)cc2)cc2cc(-c3nc(-c4ccccc4)nc(-c4ccccc4)n3)ccc12]

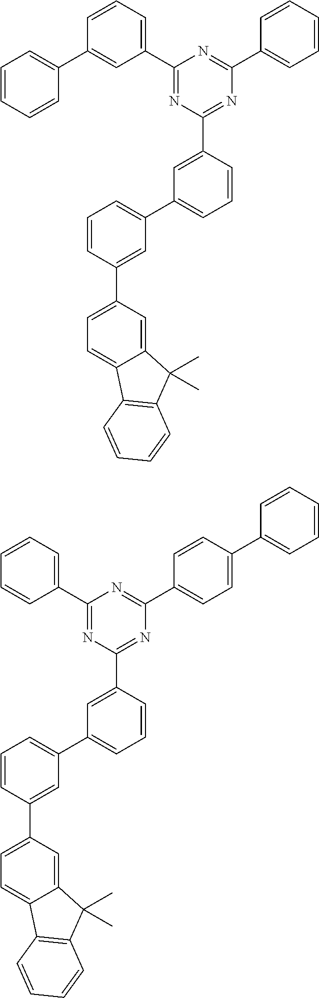 [SMILES: CC1(C)c2ccccc2-c2ccc(-c3cccc(-c4cccc(-c5nc(-c6ccccc6)nc(-c6cccc(-c7ccccc7)c6)n5)c4)c3)cc21 ; CC1(C)c2ccccc2-c2ccc(-c3cccc(-c4cccc(-c5nc(-c6ccccc6)nc(-c6ccc(-c7ccccc7)cc6)n5)c4)c3)cc21]

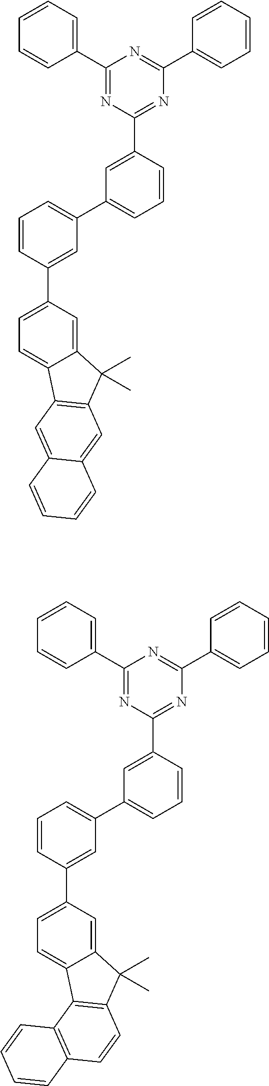 [SMILES: CC1(C)c2cc(-c3cccc(-c4cccc(-c5nc(-c6ccccc6)nc(-c6ccccc6)n5)c4)c3)ccc2-c2cc3ccccc3cc21 ; CC1(C)c2cc(-c3cccc(-c4cccc(-c5nc(-c6ccccc6)nc(-c6ccccc6)n5)c4)c3)ccc2-c2c1ccc1ccccc21]

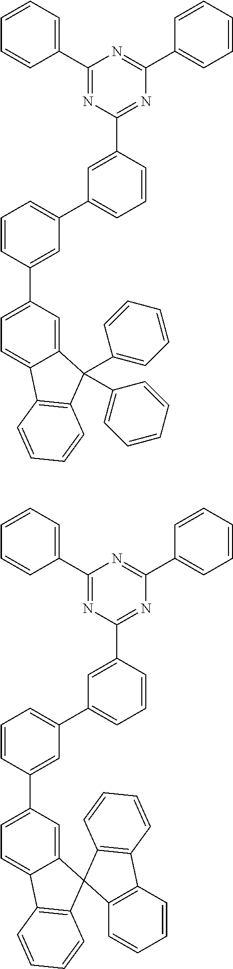 [SMILES: c1ccc(-c2nc(-c3ccccc3)nc(-c3cccc(-c4cccc(-c5ccc6c(c5)C(c5ccccc5)(c5ccccc5)c5ccccc5-6)c4)c3)n2)cc1 ; c1ccc(-c2nc(-c3ccccc3)nc(-c3cccc(-c4cccc(-c5ccc6c(c5)C5(c7ccccc7-c7ccccc75)c5ccccc5-6)c4)c3)n2)cc1]

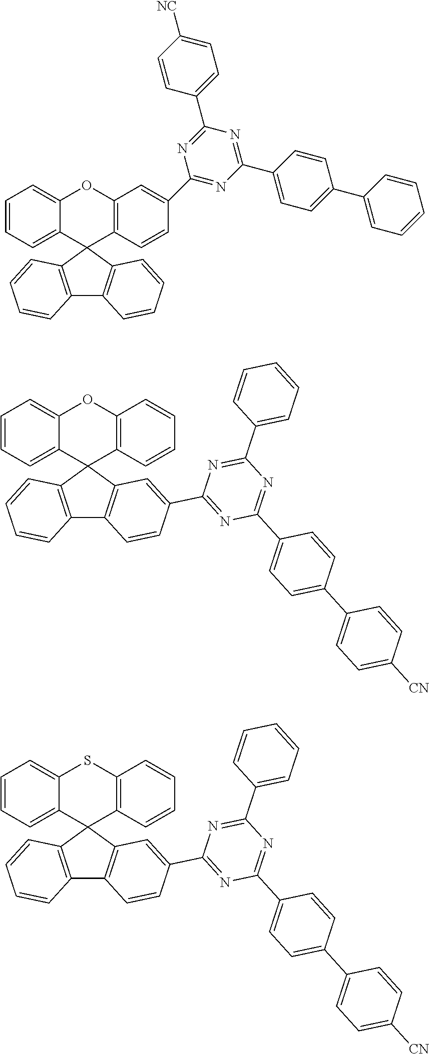 [SMILES: N#Cc1ccc(-c2nc(-c3ccc(-c4ccccc4)cc3)nc(-c3ccc4c(c3)Oc3ccccc3C43c4ccccc4-c4ccccc43)n2)cc1 ; N#Cc1ccc(-c2ccc(-c3nc(-c4ccccc4)nc(-c4ccc5c(c4)C4(c6ccccc6Oc6ccccc64)c4ccccc4-5)n3)cc2)cc1 ; N#Cc1ccc(-c2ccc(-c3nc(-c4ccccc4)nc(-c4ccc5c(c4)C4(c6ccccc6Sc6ccccc64)c4ccccc4-5)n3)cc2)cc1]

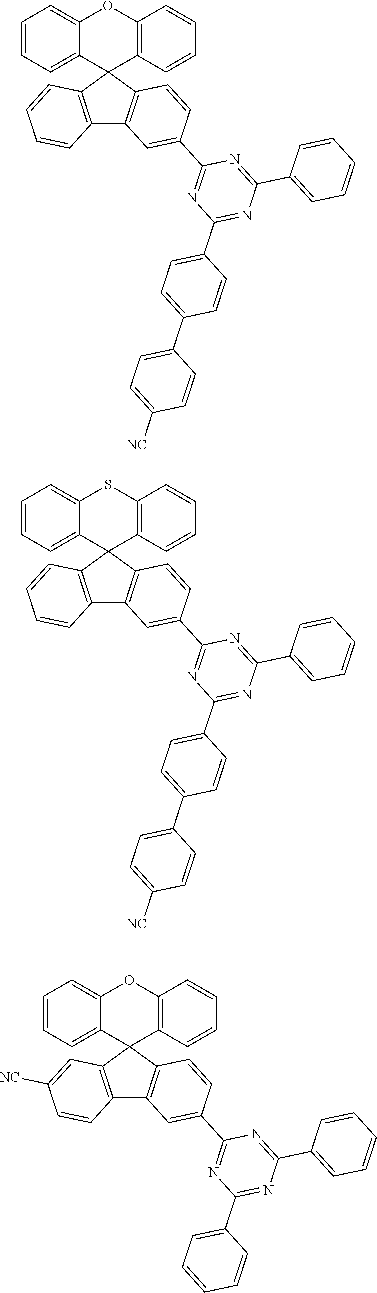 [SMILES: N#Cc1ccc(-c2ccc(-c3nc(-c4ccccc4)nc(-c4ccc5c(c4)-c4ccccc4C54c5ccccc5Oc5ccccc54)n3)cc2)cc1 ; N#Cc1ccc(-c2ccc(-c3nc(-c4ccccc4)nc(-c4ccc5c(c4)-c4ccccc4C54c5ccccc5Sc5ccccc54)n3)cc2)cc1 ; N#Cc1ccc2c(c1)C1(c3ccccc3Oc3ccccc31)c1ccc(-c3nc(-c4ccccc4)nc(-c4ccccc4)n3)cc1-2]

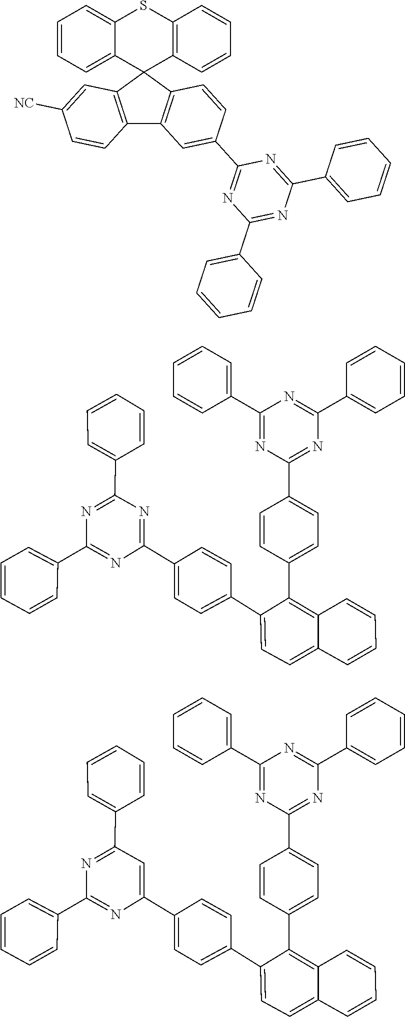

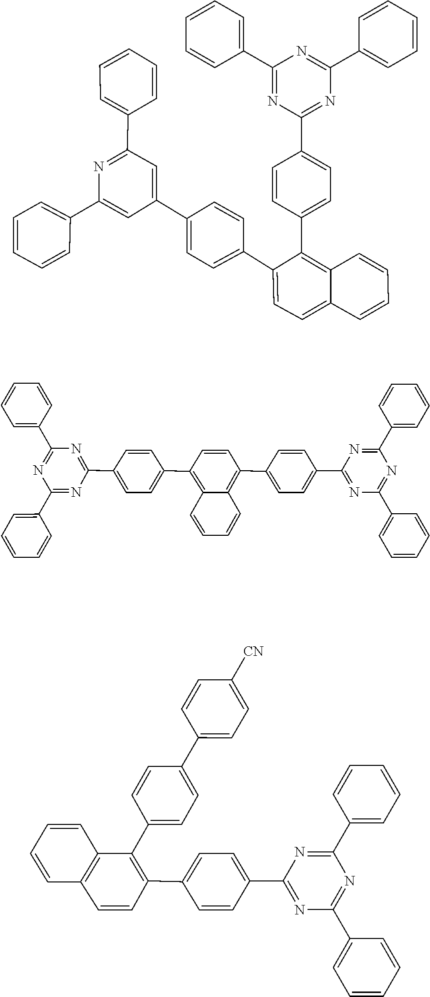 [SMILES: c1ccc(-c2cc(-c3ccc(-c4ccc5ccccc5c4-c4ccc(-c5nc(-c6ccccc6)nc(-c6ccccc6)n5)cc4)cc3)cc(-c3ccccc3)n2)cc1 ; c1ccc(-c2nc(-c3ccccc3)nc(-c3ccc(-c4ccc(-c5ccc(-c6nc(-c7ccccc7)nc(-c7ccccc7)n6)cc5)c5ccccc45)cc3)n2)cc1 ; N#Cc1ccc(-c2ccc(-c3c(-c4ccc(-c5nc(-c6ccccc6)nc(-c6ccccc6)n5)cc4)ccc4ccccc34)cc2)cc1]

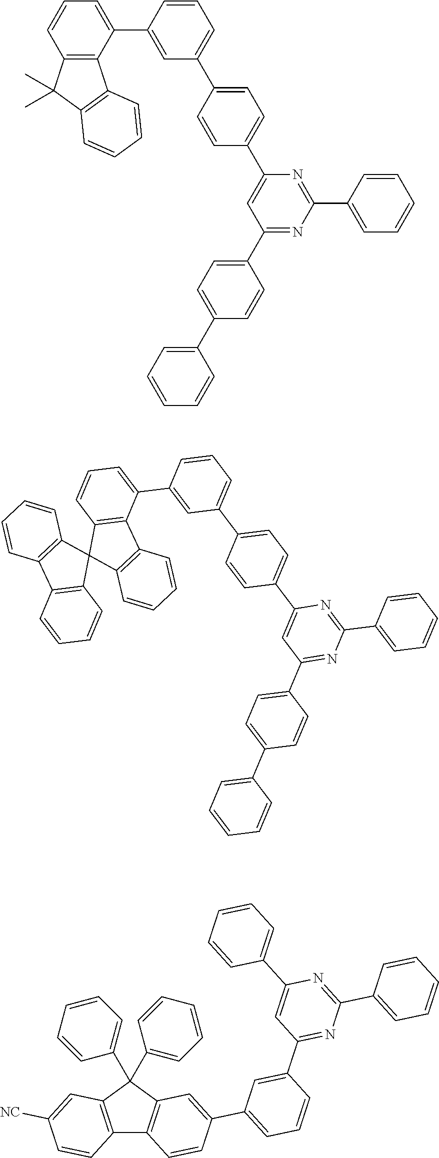 [SMILES: CC1(C)c2ccccc2-c2c(-c3cccc(-c4ccc(-c5cc(-c6ccc(-c7ccccc7)cc6)nc(-c6ccccc6)n5)cc4)c3)cccc21 ; c1ccc(-c2ccc(-c3cc(-c4ccc(-c5cccc(-c6cccc7c6-c6ccccc6C76c7ccccc7-c7ccccc76)c5)cc4)nc(-c4ccccc4)n3)cc2)cc1 ; N#Cc1ccc2c(c1)C(c1ccccc1)(c1ccccc1)c1cc(-c3cccc(-c4cc(-c5ccccc5)nc(-c5ccccc5)n4)c3)ccc1-2]

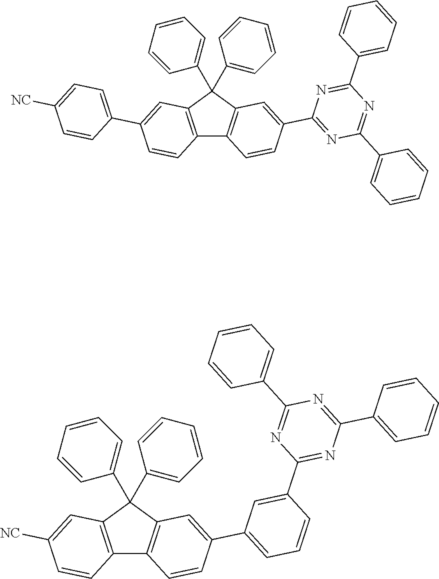 [SMILES: N#Cc1ccc(-c2ccc3c(c2)C(c2ccccc2)(c2ccccc2)c2cc(-c4nc(-c5ccccc5)nc(-c5ccccc5)n4)ccc2-3)cc1 ; N#Cc1ccc2c(c1)C(c1ccccc1)(c1ccccc1)c1cc(-c3cccc(-c4nc(-c5ccccc5)nc(-c5ccccc5)n4)c3)ccc1-2]

An embodiment may be an organic light emitting diode including the light emitting layer as the organic layer.

Another embodiment may be an organic light emitting diode including a light emitting layer and a hole transport region as the organic layer.

Another embodiment may be an organic light emitting diode including a light emitting layer and an electron transport region as the organic layer.

An organic light emitting diode according to an embodiment includes a hole transport region 140 and an electron transport region 150 in addition to the light emitting layer 130 as the organic layer 105, as shown in FIG. 1.

On the other hand, an organic light emitting diode may further include an electron injection layer (not shown), a hole injection layer (not shown), etc. in addition to the light emitting layer as the organic layer.

The organic light emitting diodes 100 may be manufactured by forming an anode or a cathode on a substrate, and then forming an organic layer by a dry film method such as vacuum deposition, sputtering, plasma plating and ion plating, and forming a cathode or an anode thereon.

The organic light emitting diode may be applied to an organic light emitting display device.

### MODE FOR INVENTION

Hereinafter, the embodiments are illustrated in more detail with reference to examples. However, these examples are exemplary, and the scope of claims is not limited thereto.

Hereinafter, starting materials and reactants used in Examples and Synthesis Examples were purchased from Sigma-Aldrich Co. Ltd., TCI Inc., Tokyo chemical industry, or P&H tech as far as there is no particular comment or were synthesized by known methods.

### (Preparation of Compound for Organic Optoelectronic Device)

### Synthesis Example 1: Synthesis of Compound 1-1

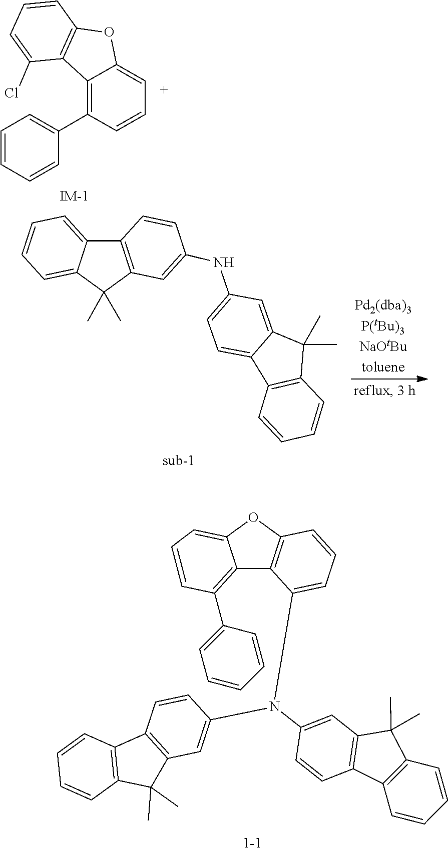

IM-1 (15.0 g, 53.8 mmol) was dissolved in toluene (180 mL), and sub-1 (21.6 g, 53.8 mmol), NaOtBu (6.98 g, 72.6 mmol), Pd₂(dba)₃ (2.96 g, 3.23 mmol), and P(t-Bu)₃ (4.61 mL, 9.7 mmol) were added thereto and then, refluxed for 3 hours. When a reaction was completed, an organic layer, which was extracted with CH₂Cl₂ and water therefrom, was dried with MgSO₄, concentrated, separated through a silica gel column, and recrystallized, obtaining 14.5 g (Yield: 42%) of Compound 1-1.

### Synthesis Example 2: Synthesis of Compound 1-4

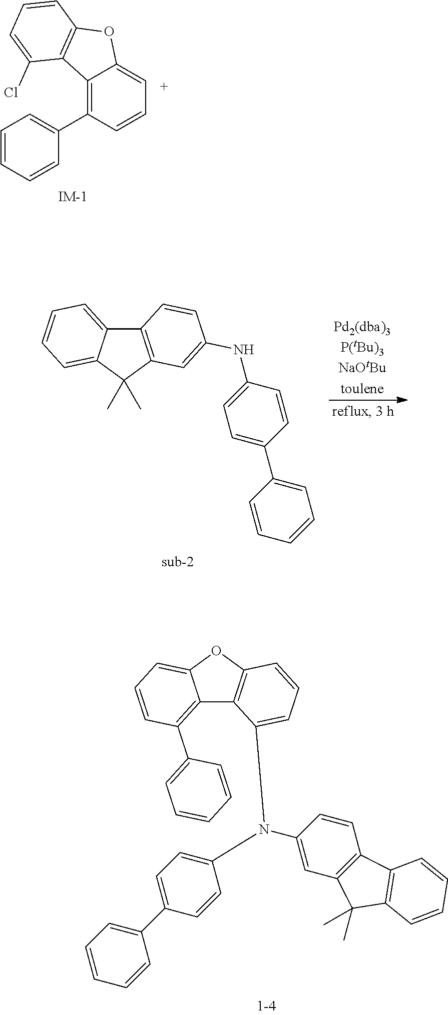

IM-1 (15.0 g, 53.8 mmol) was dissolved in toluene (180 mL), and sub-2 (19.4 g, 53.8 mmol), NaOtBu (6.98 g, 72.6 mmol), Pd₂(dba)₃ (2.96 g, 3.23 mmol), and P(t-Bu)₃ (4.61 mL, 9.7 mmol) were added thereto and then, refluxed for 3 hours. When a reaction was completed, an organic layer, which was extracted with CH₂Cl₂ and water therefrom, was dried with MgSO₄, concentrated, separated through a silica gel column, and recrystallized, obtaining 15.6 g (Yield: 48%) of Compound 1-4.

### Synthesis Example 3: Synthesis of Compound 1-10

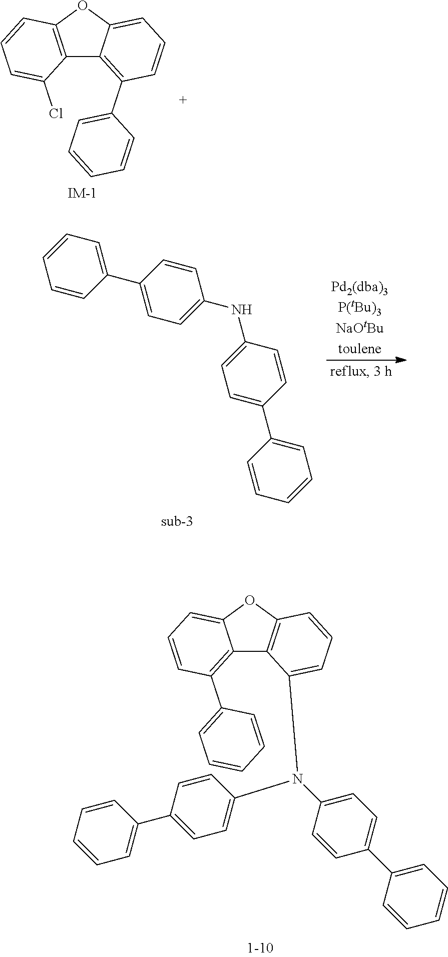

IM-1 (15.0 g, 53.8 mmol) was dissolved in toluene (180 mL), and sub-3 (17.3 g, 53.8 mmol), NaOtBu (6.98 g, 72.6 mmol), Pd₂(dba)₃ (2.96 g, 3.23 mmol), and P(t-Bu)₃ (4.61 mL, 9.7 mmol) were added thereto and then, refluxed for 3 hours. When a reaction was completed, an organic layer, which was extracted with CH₂Cl₂ and water therefrom, was dried with MgSO₄, concentrated, separated through a silica gel column, and recrystallized, obtaining 13.4 g (Yield: 44%) of Compound 1-10.

### Synthesis Example 4: Synthesis of Compound 1-26

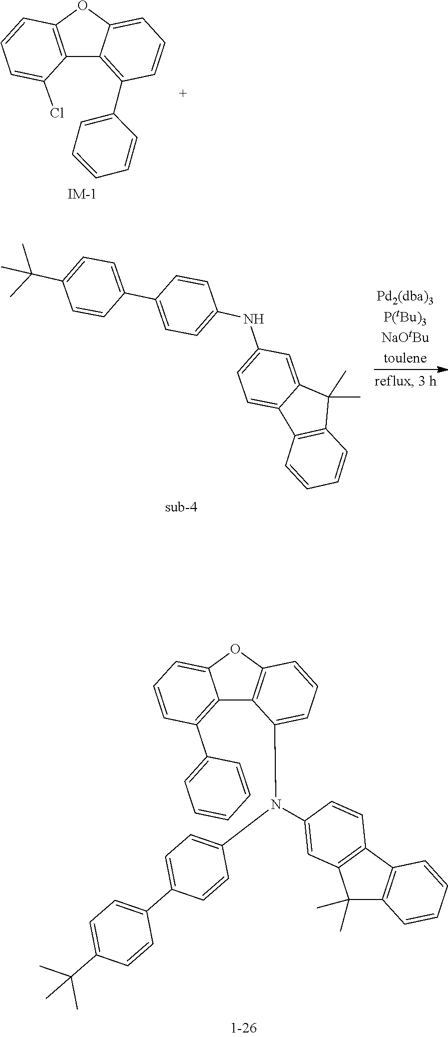

IM-1 (15.0 g, 53.8 mmol) was dissolved in toluene (180 mL), and sub-4 (22.5 g, 53.8 mmol), NaOtBu (6.98 g, 72.6 mmol), Pd₂(dba)₃ (2.96 g, 3.23 mmol), and P(t-Bu)₃ (4.61 mL, 9.7 mmol) were added thereto and then, refluxed for 3 hours. When a reaction was completed, an organic layer, which was extracted with CH₂Cl₂ and water therefrom, was dried with MgSO₄, concentrated, separated through a silica gel column, and recrystallized, obtaining 15.1 g (Yield: 42%) of Compound 1-26.

### Synthesis Example 5: Synthesis of Compound 1-27

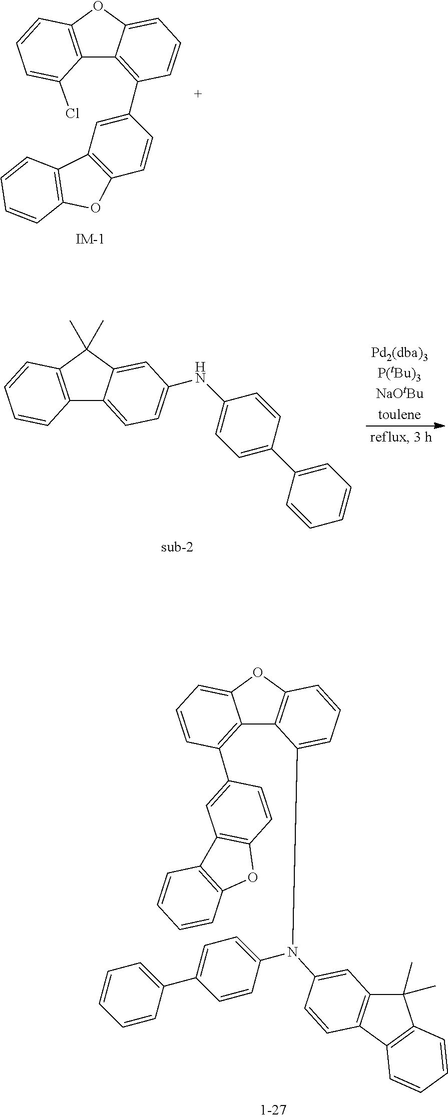

IM-2 (15.0 g, 40.8 mmol) was dissolved in toluene (135 mL), and sub-2 (14.7 g, 40.8 mmol), NaOtBu (5.29 g, 55.1 mmol), Pd₂(dba)₃ (2.24 g, 2.45 mmol), and P(t-Bu)₃ (3.50 mL, 7.3 mmol) were added thereto and then, refluxed for 3 hours. When a reaction was completed, an organic layer, which was extracted with CH₂Cl₂ and water therefrom, was dried with MgSO₄, concentrated, separated through a silica gel column, and recrystallized, obtaining 12.4 g (Yield: 44%) of Compound 1-27.

### Synthesis Example 6: Synthesis of Compound 1-28

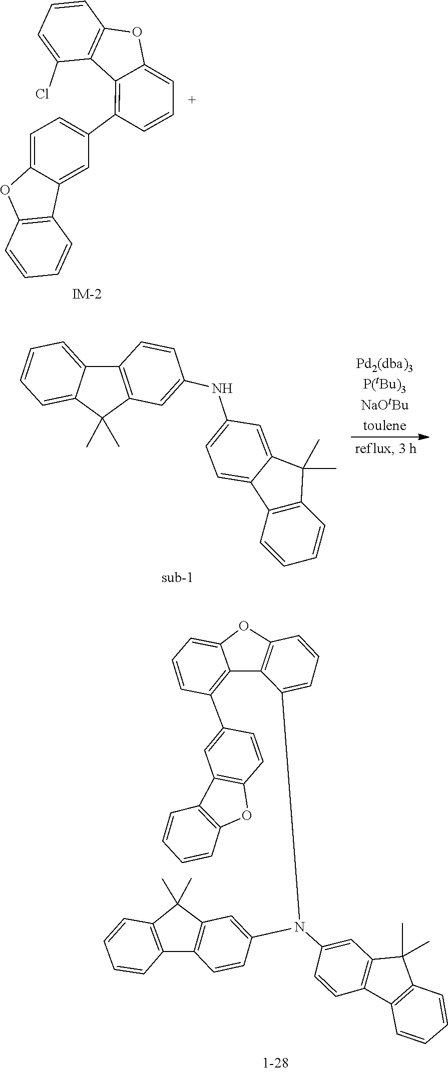

IM-2 (15.0 g, 40.8 mmol) was dissolved in toluene (135 mL), and sub-1 (16.4 g, 40.8 mmol), NaOtBu (5.29 g, 55.1 mmol), Pd₂(dba)₃ (2.24 g, 2.45 mmol), and P(t-Bu)₃ (3.50 mL, 7.3 mmol) were added thereto and then, refluxed for 3 hours. When a reaction was completed, an organic layer, which was extracted with CH₂Cl₂ and water therefrom, was dried with MgSO₄, concentrated, separated through a silica gel column, and recrystallized, obtaining 11.8 g (Yield: 40%) of Compound 1-28.

### Comparative Synthesis Example 1: Synthesis of Compound F-1

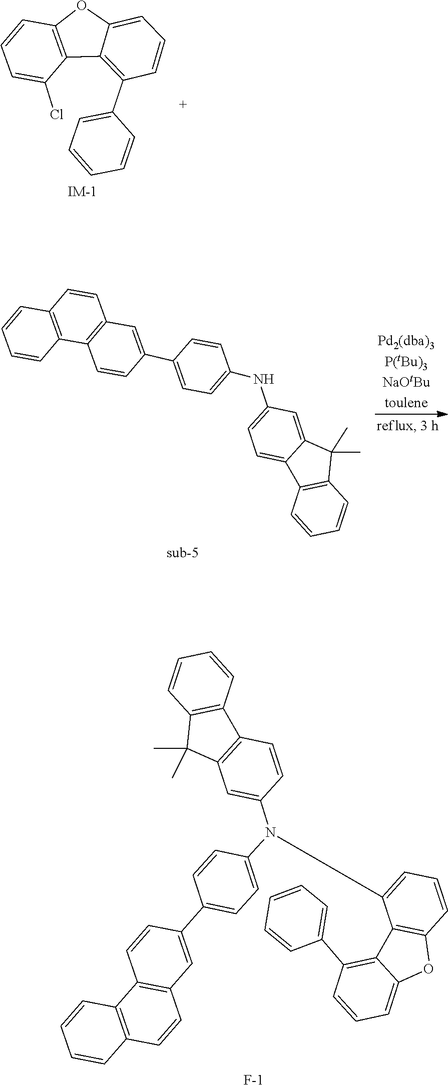

IM-1 (15.0 g, 53.8 mmol) was dissolved in toluene (180 mL), and sub-5 (24.9 g, 53.8 mmol), NaOtBu (6.98 g, 72.6 mmol), Pd₂(dba)₃ (2.96 g, 3.23 mmol), and P(t-Bu)₃ (4.61 mL, 9.7 mmol) were added thereto and then, refluxed for 3 hours. When a reaction was completed, an organic layer, which was extracted with CH₂Cl₂ and water therefrom, was dried with MgSO₄, concentrated, separated through a silica gel column, and recrystallized, obtaining 16.1 g (Yield: 41%) of Compound F-1.

### Comparative Synthesis Example 2: Synthesis of Compound F-2

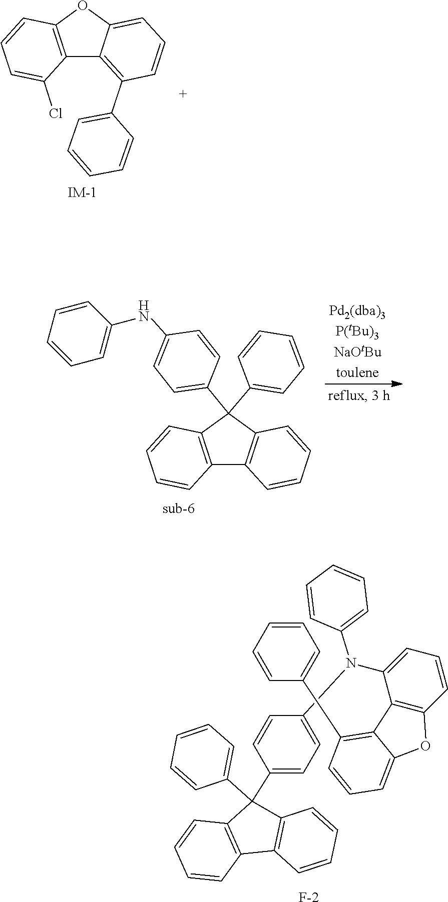

IM-1 (15.0 g, 53.8 mmol) was dissolved in toluene (180 mL), and sub-5 (24.9 g, 53.8 mmol), NaOtBu (6.98 g, 72.6 mmol), Pd₂(dba)₃ (2.96 g, 3.23 mmol), and P(t-Bu)₃ (4.61 mL, 9.7 mmol) were added thereto and then, refluxed for 3 hours. When a reaction was completed, an organic layer, which was extracted with CH₂Cl₂ and water therefrom, was dried with MgSO₄, concentrated, separated through a silica gel column, and recrystallized, obtaining 17.2 g (Yield: 49%) of Compound F-2.

### (Manufacturing of Organic Light Emitting Diode)

### Example 1

A glass substrate coated with a thin film of ITO/Ag/ITO was ultrasonically cleaned with distilled water. After washing with the distilled water, the glass substrate was washed with a solvent such as acetone, isopropyl alcohol, and the like ultrasonically and dried and then, moved to a plasma cleaner, cleaned by using oxygen plasma for 10 minutes, and moved to a vacuum depositor. This prepared ITO/Ag/ITO (reflective electrode) was used as an anode, Compound A doped with 3% NDP-9 (Novaled GmbH) was vacuum-deposited on the ITO/Ag/ITO substrate to form a 100 Å-thick hole injection layer, and Compound A is deposited on the hole injection layer to a thickness of 1350 Å to form a hole transport layer. Compound 1-1 obtained in Synthesis Example 1 was deposited on the hole transport layer to a thickness of 335 Å to form a hole transport auxiliary layer. On the hole transport auxiliary layer, 90 wt % of Host H1 (40%) and Host H2 (60%) were used as hosts, and 10 wt % of GD was doped as a dopant to form a 380 Å-thick light emitting layer by vacuum deposition. Subsequently, Compound C was deposited on the light emitting layer to a thickness of 50 Å to form an electron transport auxiliary layer, and Compound D and Liq were simultaneously vacuum-deposited in a weight ratio of 1:1 to form a 310 Å-thick electron transport layer. An organic light emitting diode was manufactured by sequentially vacuum-depositing Yb, AgMg on the electron transport layer to form a cathode.

The organic light emitting diode was manufactured to have a structure of ITO/Ag/ITO/Compound A (3% NDP-9 doping, 100 Å)/Compound A (1350 Å)/hole transport auxiliary layer (335 Å)/light emitting layer [Host (Host H1:Host H2=40 wt %: 60 wt %):GD=90 wt %: 10 wt %](380 Å)/Compound C (50 Å)/Compound D:Liq (310 Å)/Yb/AgMg.
Compound A: N-(biphenyl-4-yl)-9,9-dimethyl-N-(4-(9-phenyl-9H-carbazol-3-yl)phenyl)-9H-fluoren-2-amine
Compound C: 2-(3′-(9,9-dimethyl-9H-fluoren-2-yl)-[1,1′-biphenyl]-3-yl)-4,6-diphenyl-1,3,5-triazine
Compound D: 6,6′-(naphthalene-1,2-diylbis(4,1-phenylene))bis(2,4-diphenyl-1,3,5-triazine)
HostH1: 2-([1,1′-biphenyl]-4-yl)-4-phenyl-6-(3-(triphenylen-2-yl)phenyl)-1,3,5-triazine
Host H2: 9,9″-diphenyl-9H,9″H-3,3′:9′,3″-tercarbazole GD:

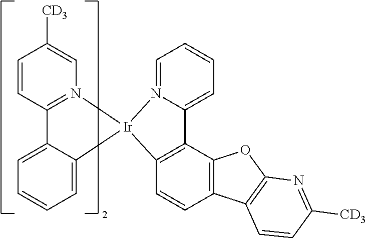

### Examples 2 to 6 and Comparative Examples 1 and 2

The diodes of Examples 2 to 6 and Comparative Examples 1 and 2 were manufactured in the same manner as Example 1, except that the compositions of the hole transport auxiliary layer were changed as shown in Table 1.

### Evaluation

(1) Measurement of Current Density Change Depending on Voltage Change
The obtained organic light emitting diodes were measured regarding a current value flowing in the unit diode, while increasing the voltage from 0 V to 10 V using a current-voltage meter (Keithley 2400), and the measured current value was divided by area to provide the results.
(2) Measurement of Luminance Change Depending on Voltage Change
Luminance was measured by using a luminance meter (Minolta Cs-1000A), while the voltage of the organic light emitting diodes was increased from 0 V to 10 V.
(3) Measurement of Driving Voltage
A driving voltage of each diode was measured using a current-voltage meter (Keithley 2400) at 15 mA/cm².

The relative values based on the driving voltage of Comparative Example 1 are shown in Table 1.
(4) Measurement of Life-Span

Time when each current efficiency (cd/A) was reduced to 97%, while maintaining luminance (cd/m²) at 24000 cd/m², was measured as a life-span.

The relative values based on the life-span measurement values of Comparative Example 1 are shown in Table 1.

TABLE 1

| | | Driving | Life-span |
|---|---|---|---|
| | Hole transport | voltage | characteristics |
| | auxiliary layer | (%) | (%) |
| Example 1 | 1-1 | 87 | 135 |
| Example 2 | 1-4 | 87 | 136 |
| Example 3 | 1-10 | 94 | 150 |
| Example 4 | 1-26 | 91 | 135 |
| Example 5 | 1-27 | 90 | 145 |
| Example 6 | 1-28 | 84 | 162 |
| Comparative Example 1 | F-1 | 100 | 100 |
| Comparative Example 2 | F-2 | 94 | 119 |

Referring to Table 1, the organic light emitting diodes to which the compounds according to Examples of the present invention are applied have significantly improved driving voltage and life-span characteristics compared to the organic light emitting diodes according to Comparative Examples.

While this invention has been described in connection with what is presently considered to be practical example embodiments, it is to be understood that the invention is not limited to the disclosed embodiments. On the contrary, it is intended to cover various modifications and equivalent arrangements included within the spirit and scope of the appended claims.

1. A compound for an organic optoelectronic device represented by Chemical Formula 1: 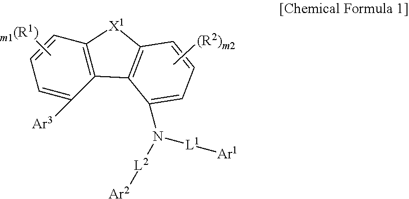
wherein, in Chemical Formula 1,
X¹ is O or S
R¹ and R² are each independently hydrogen, deuterium, a cyano group, a substituted or unsubstituted C1 to C20 alkyl group, a substituted or unsubstituted C6 to C20 aryl group, or a combination thereof,
Ar¹ and Ar² are each independently a substituted or unsubstituted phenyl group, a substituted or unsubstituted biphenyl group, a substituted or unsubstituted terphenyl group, a substituted or unsubstituted naphthyl group, a substituted or unsubstituted triphenylene group, a substituted or unsubstituted 1-fluorenyl group, a substituted or unsubstituted 2-fluorenyl group, a substituted or unsubstituted 3-fluorenyl group, a substituted or unsubstituted 4-fluorenyl group, a substituted or unsubstituted 9,9′-spirobifluorenyl group, a substituted or unsubstituted dibenzofuranyl group, a substituted or unsubstituted dibenzothiophenyl group, a substituted or unsubstituted dibenzosilolyl group, a substituted or unsubstituted spirocyclopentane-1,9′-fluorenyl group, a substituted or unsubstituted spirocyclohexane-1,9′-fluorenyl group, or a substituted or unsubstituted spirofluorene-9,9′-xanthenyl group,
Ar³ is a substituted or unsubstituted C6 to C20 aryl group or a substituted or unsubstituted C2 to C20 heterocyclic group,
L¹ and L² are each independently a single bond, a substituted or unsubstituted C6 to C20 arylene group, a substituted or unsubstituted C2 to C30 heterocyclic group, or a combination thereof,
m1 and m2 are each independently one of integers of 1 to 3, and
“substituted” refers to replacement of at least one hydrogen by deuterium, C1 to C5 alkyl group, a C6 to C12 aryl group, or a cyano group.

2. The compound for the organic optoelectronic device as claimed in claim 1, wherein L¹ and L² are each independently a single bond, a substituted or unsubstituted phenylene group, or a substituted or unsubstituted biphenylene group.

3. The compound for the organic optoelectronic device as claimed in claim 1, wherein L¹-Ar¹ and L²-Ar² are each independently selected from the substituents listed in Group I: 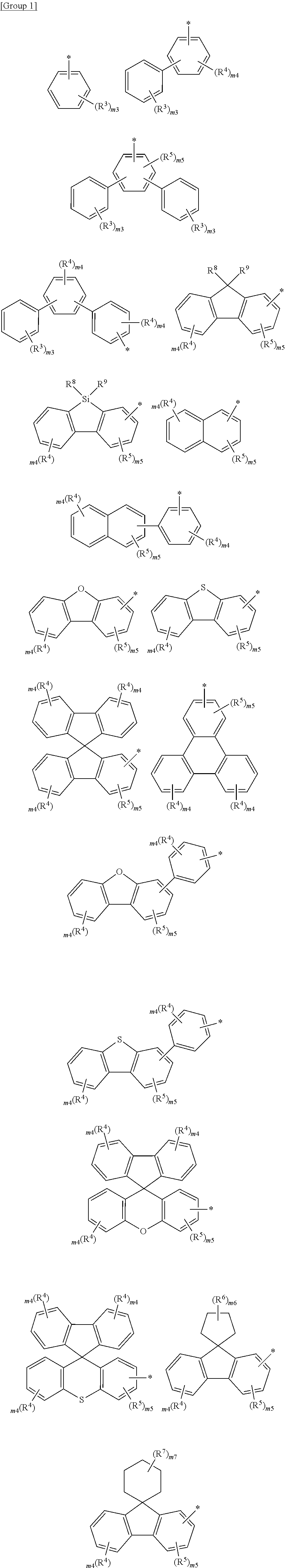
wherein, in Group I,
R³ to R⁷ are each independently hydrogen, deuterium, a cyano group, a substituted or unsubstituted C1 to C10 alkyl group, a substituted or unsubstituted C3 to C10 cycloalkyl group, or a substituted or unsubstituted C6 to C12 aryl group,
R⁸ and R⁹ are each independently a substituted or unsubstituted C1 to C10 alkyl group or a substituted or unsubstituted C6 to C12 aryl group,
m3 is one of integers of 1 to 5,
m4 is one of integers of 1 to 4,
m5 is one of integers of 1 to 3,
m6 is one of integers of 1 to 8,
m7 is one of integers of 1 to 10, and
* is a linking point.

4. The compound for the organic optoelectronic device as claimed in claim 1, wherein Ar³ is a substituted or unsubstituted phenyl group, a substituted or unsubstituted biphenyl group, a substituted or unsubstituted naphthyl group, a substituted or unsubstituted dibenzofuranyl group, or a substituted or unsubstituted dibenzothiophenyl group.

5. The compound for the organic optoelectronic device as claimed in claim 1, wherein the compound is selected from the compounds listed in Group 1:

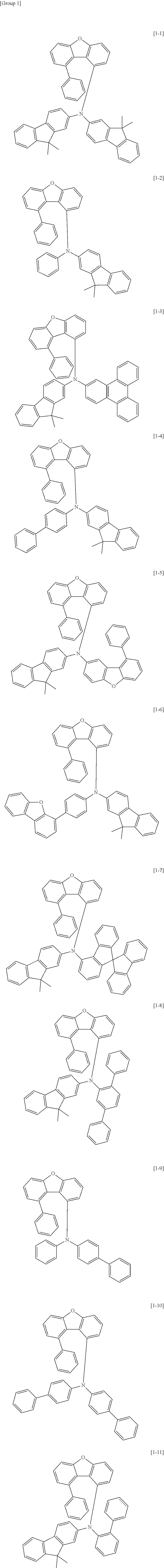

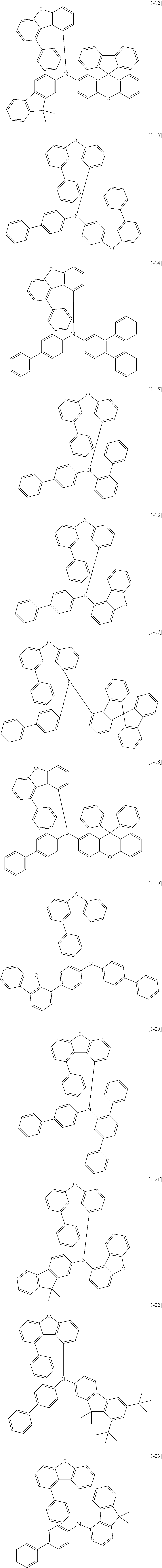

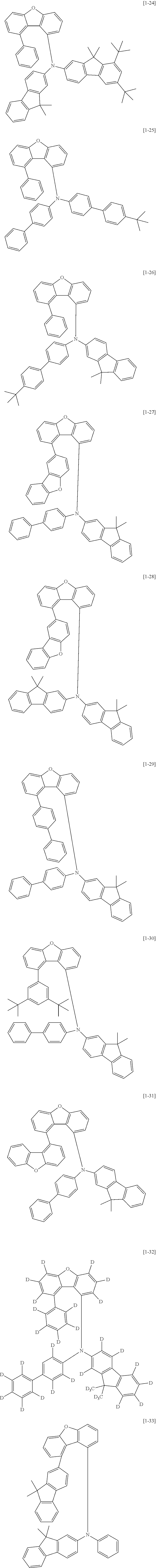

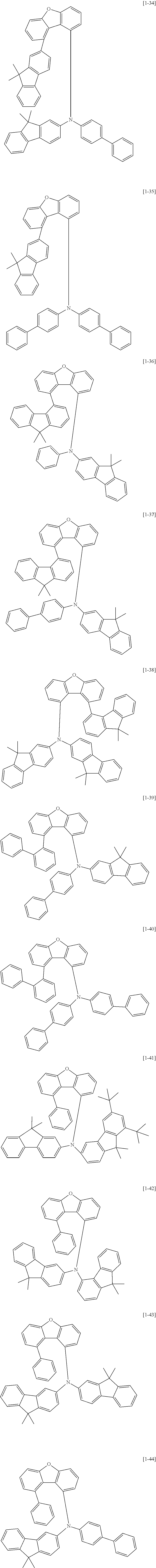

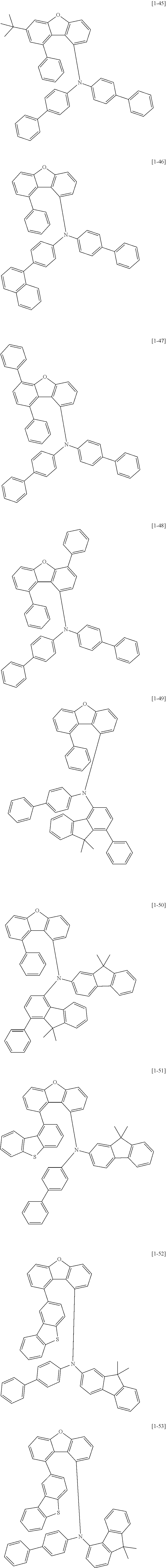

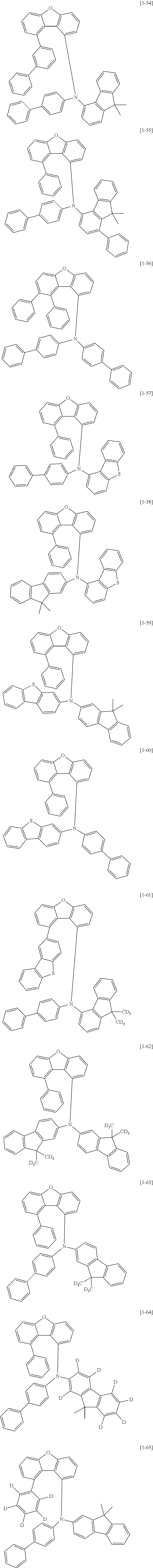

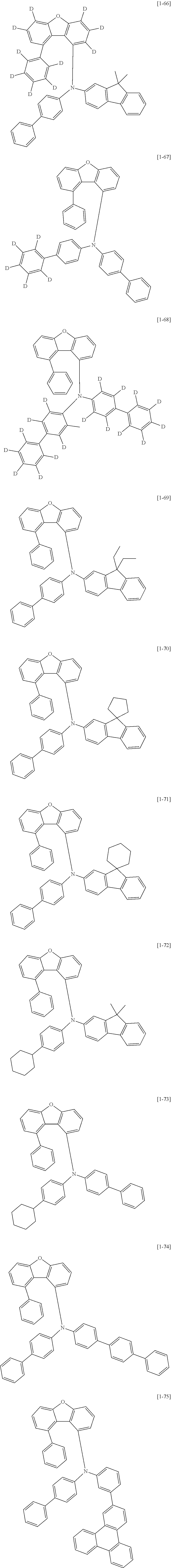

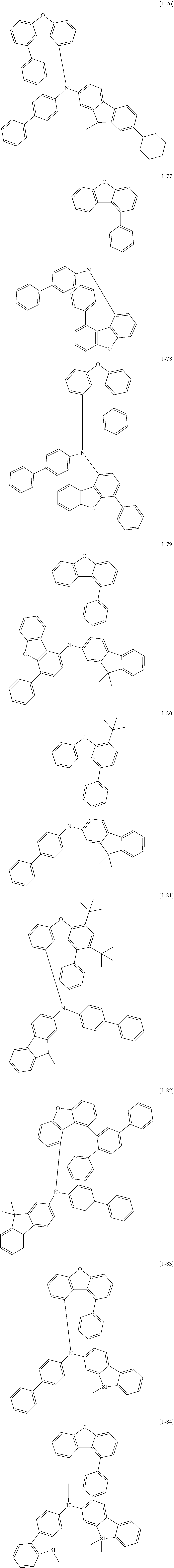

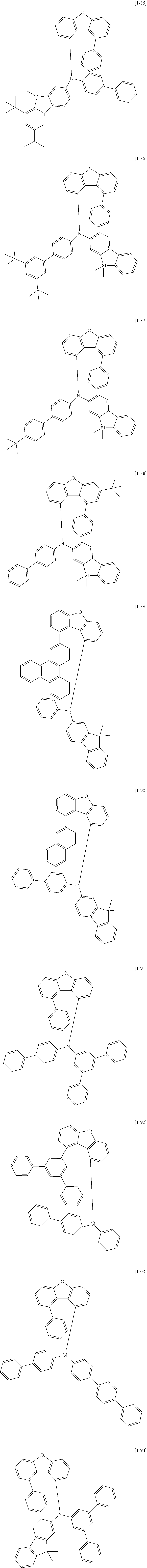

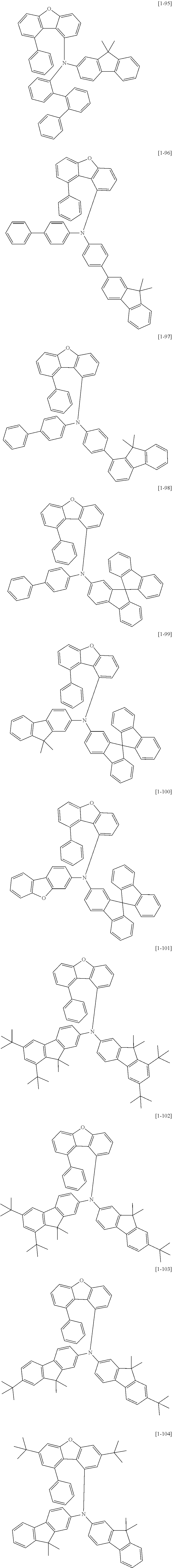

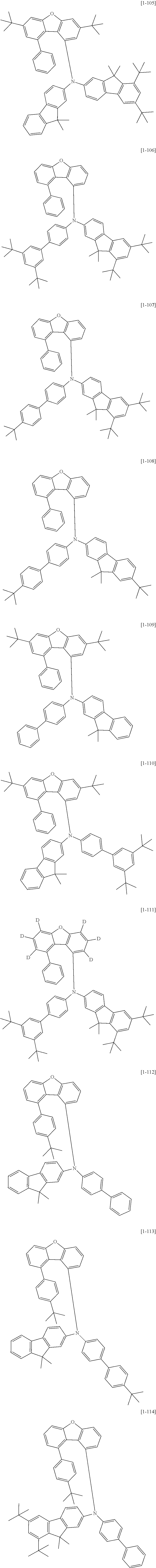

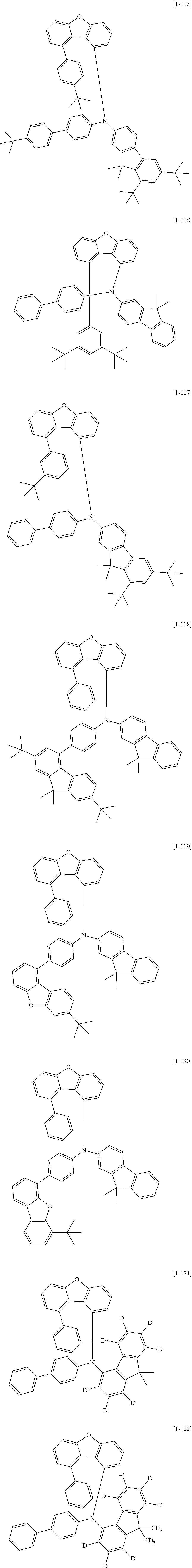

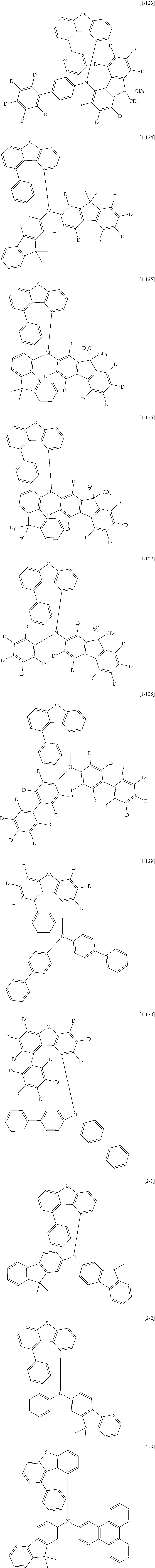

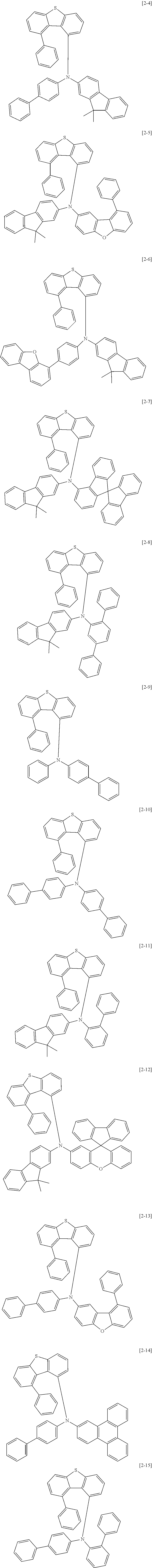

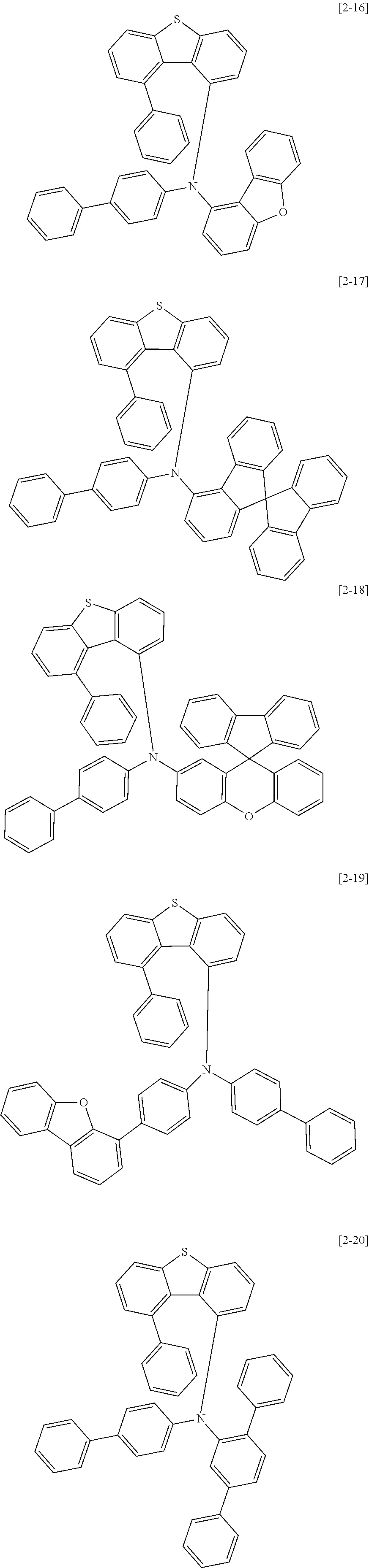 [SMILES: c1ccc(-c2ccc(N(c3cccc4oc5ccccc5c34)c3cccc4sc5cccc(-c6ccccc6)c5c34)cc2)cc1 ; c1ccc(-c2ccc(N(c3cccc4c3-c3ccccc3C43c4ccccc4-c4ccccc43)c3cccc4sc5cccc(-c6ccccc6)c5c34)cc2)cc1 ; c1ccc(-c2ccc(N(c3ccc4c(c3)C3(c5ccccc5O4)c4ccccc4-c4ccccc43)c3cccc4sc5cccc(-c6ccccc6)c5c34)cc2)cc1 ; c1ccc(-c2ccc(N(c3ccc(-c4cccc5c4oc4ccccc45)cc3)c3cccc4sc5cccc(-c6ccccc6)c5c34)cc2)cc1 ; c1ccc(-c2ccc(N(c3cc(-c4ccccc4)ccc3-c3ccccc3)c3cccc4sc5cccc(-c6ccccc6)c5c34)cc2)cc1]

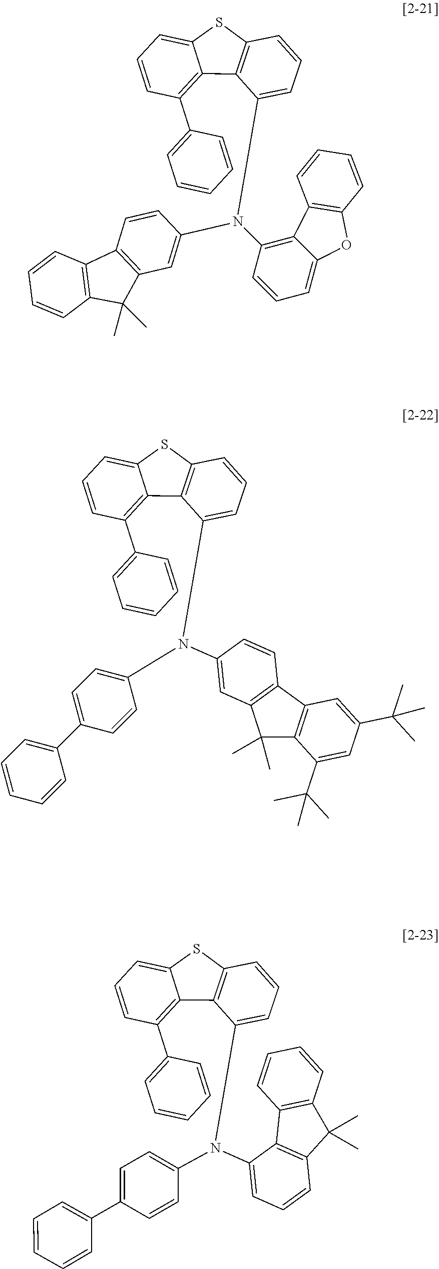 [SMILES: CC1(C)c2ccccc2-c2ccc(N(c3cccc4oc5ccccc5c34)c3cccc4sc5cccc(-c6ccccc6)c5c34)cc21 ; CC(C)(C)c1cc2c(c(C(C)(C)C)c1)C(C)(C)c1cc(N(c3ccc(-c4ccccc4)cc3)c3cccc4sc5cccc(-c6ccccc6)c5c34)ccc1-2 ; CC1(C)c2ccccc2-c2c(N(c3ccc(-c4ccccc4)cc3)c3cccc4sc5cccc(-c6ccccc6)c5c34)cccc21]

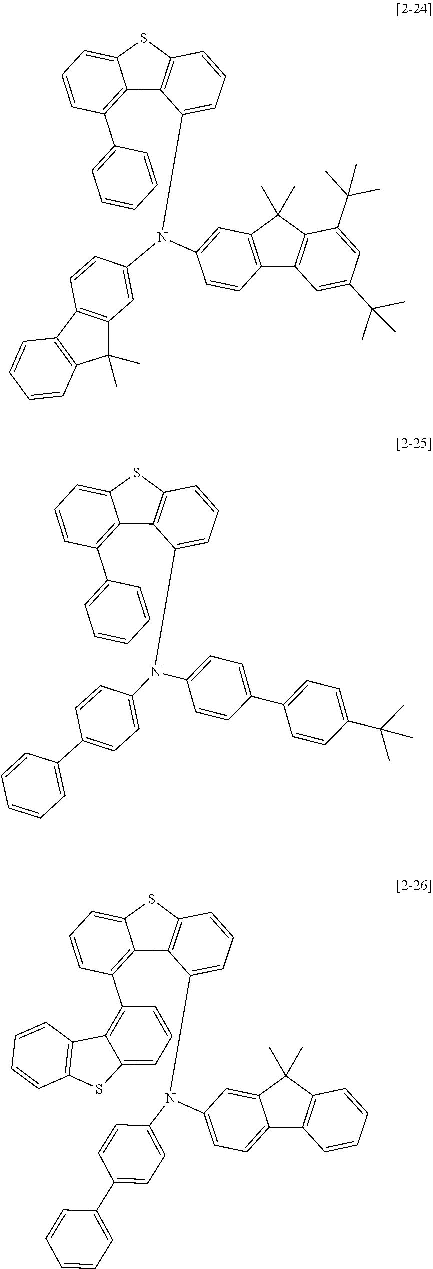 [SMILES: CC(C)(C)c1cc2c(c(C(C)(C)C)c1)C(C)(C)c1cc(N(c3ccc4c(c3)C(C)(C)c3ccccc3-4)c3cccc4sc5cccc(-c6ccccc6)c5c34)ccc1-2 ; CC(C)(C)c1ccc(-c2ccc(N(c3ccc(-c4ccccc4)cc3)c3cccc4sc5cccc(-c6ccccc6)c5c34)cc2)cc1 ; CC1(C)c2ccccc2-c2ccc(N(c3ccc(-c4ccccc4)cc3)c3cccc4sc5cccc(-c6cccc7sc8ccccc8c67)c5c34)cc21]

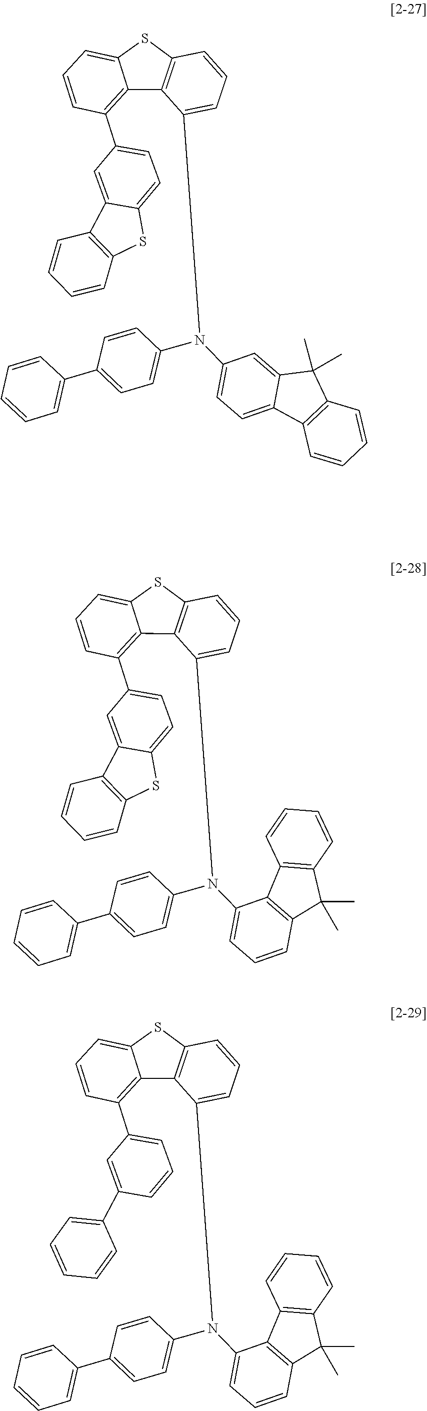 [SMILES: CC1(C)c2ccccc2-c2ccc(N(c3ccc(-c4ccccc4)cc3)c3cccc4sc5cccc(-c6ccc7sc8ccccc8c7c6)c5c34)cc21 ; CC1(C)c2ccccc2-c2c(N(c3ccc(-c4ccccc4)cc3)c3cccc4sc5cccc(-c6ccc7sc8ccccc8c7c6)c5c34)cccc21 ; CC1(C)c2ccccc2-c2c(N(c3ccc(-c4ccccc4)cc3)c3cccc4sc5cccc(-c6cccc(-c7ccccc7)c6)c5c34)cccc21]

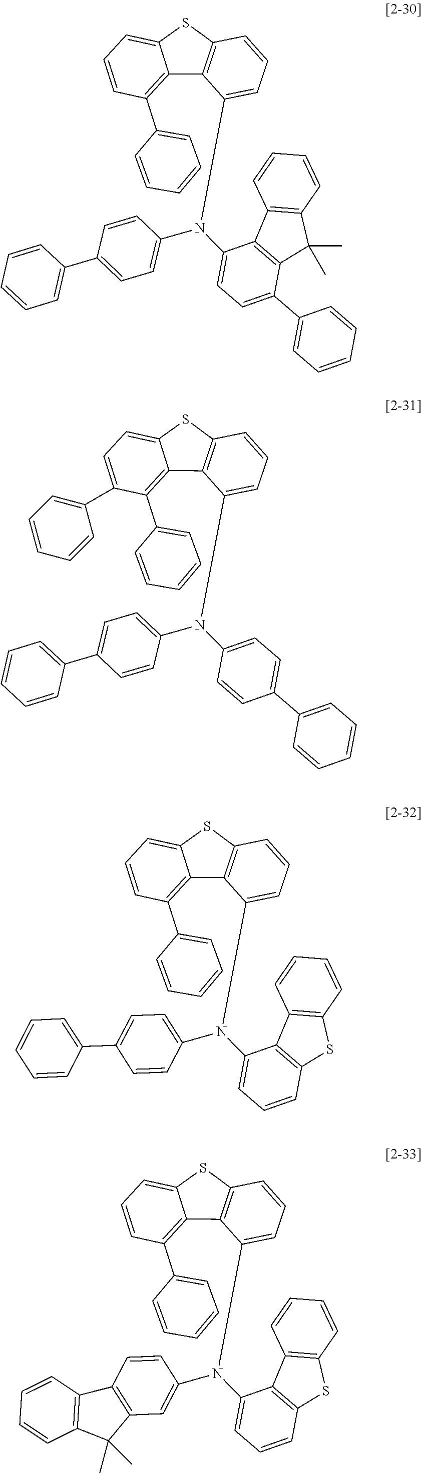 [SMILES: CC1(C)c2ccccc2-c2c(N(c3ccc(-c4ccccc4)cc3)c3cccc4sc5cccc(-c6ccccc6)c5c34)ccc(-c3ccccc3)c21 ; c1ccc(-c2ccc(N(c3ccc(-c4ccccc4)cc3)c3cccc4sc5ccc(-c6ccccc6)c(-c6ccccc6)c5c34)cc2)cc1 ; c1ccc(-c2ccc(N(c3cccc4sc5ccccc5c34)c3cccc4sc5cccc(-c6ccccc6)c5c34)cc2)cc1 ; CC1(C)c2ccccc2-c2ccc(N(c3cccc4sc5ccccc5c34)c3cccc4sc5cccc(-c6ccccc6)c5c34)cc21]

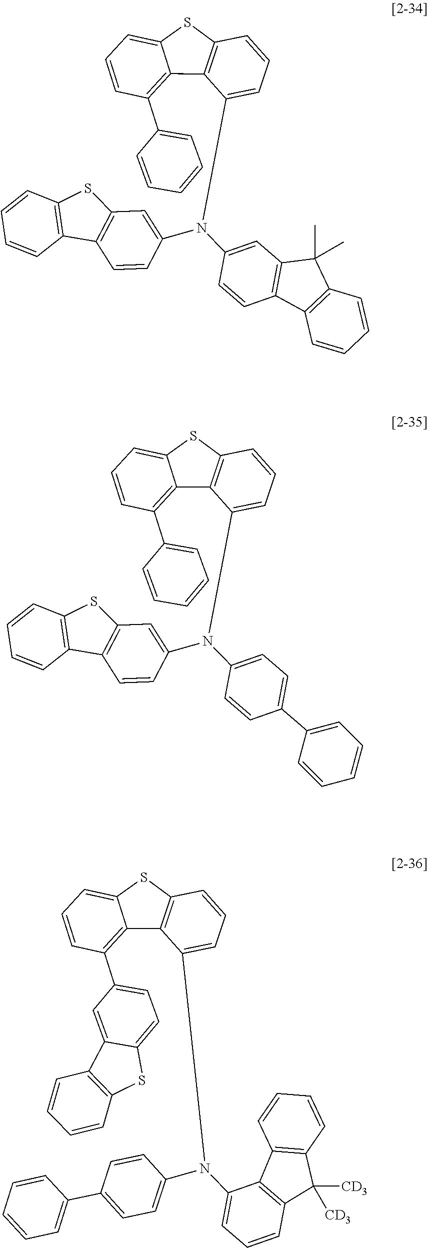 [SMILES: CC1(C)c2ccccc2-c2ccc(N(c3ccc4c(c3)sc3ccccc34)c3cccc4sc5cccc(-c6ccccc6)c5c34)cc21 ; c1ccc(-c2ccc(N(c3ccc4c(c3)sc3ccccc34)c3cccc4sc5cccc(-c6ccccc6)c5c34)cc2)cc1 ; [2H]C([2H])([2H])C1(C([2H])([2H])[2H])c2ccccc2-c2c(N(c3ccc(-c4ccccc4)cc3)c3cccc4sc5cccc(-c6ccc7sc8ccccc8c7c6)c5c34)cccc21]

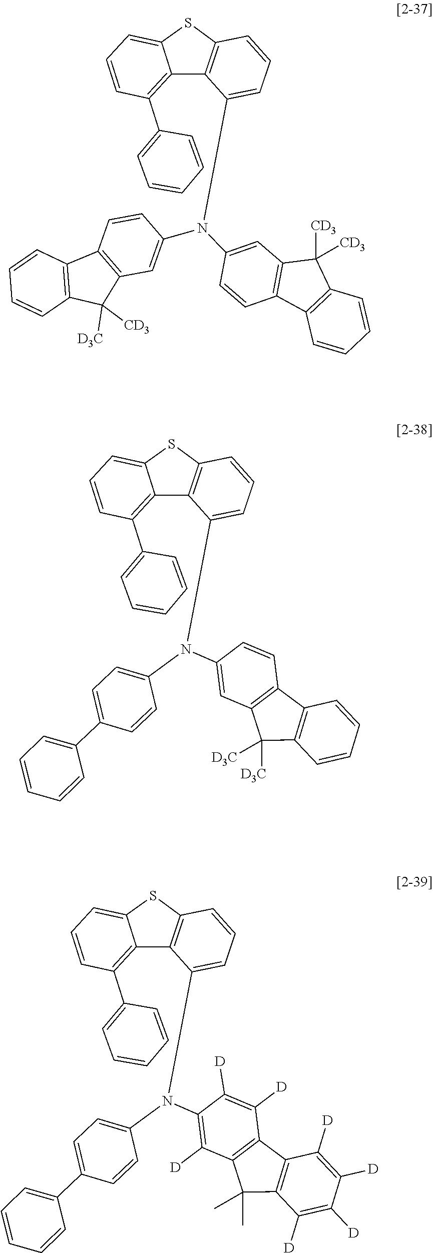 [SMILES: [2H]C([2H])([2H])C1(C([2H])([2H])[2H])c2ccccc2-c2ccc(N(c3ccc4c(c3)C(C([2H])([2H])[2H])(C([2H])([2H])[2H])c3ccccc3-4)c3cccc4sc5cccc(-c6ccccc6)c5c34)cc21 ; [2H]C([2H])([2H])C1(C([2H])([2H])[2H])c2ccccc2-c2ccc(N(c3ccc(-c4ccccc4)cc3)c3cccc4sc5cccc(-c6ccccc6)c5c34)cc21 ; [2H]c1c([2H])c([2H])c2c(c1[2H])-c1c([2H])c([2H])c(N(c3ccc(-c4ccccc4)cc3)c3cccc4sc5cccc(-c6ccccc6)c5c34)c([2H])c1C2(C)C]

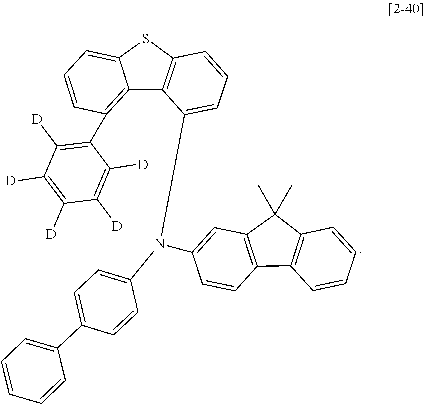 [SMILES: [2H]c1c([2H])c([2H])c(-c2cccc3sc4cccc(N(c5ccc(-c6ccccc6)cc5)c5ccc6c(c5)C(C)(C)c5ccccc5-6)c4c23)c([2H])c1[2H]]

6. An organic optoelectronic device, comprising
an anode and a cathode facing each other, and
at least one organic layer between the anode and the cathode,
wherein the organic layer comprises the compound for the organic optoelectronic device of claim 1.

7. The organic optoelectronic device as claimed in claim 6, wherein
the organic layer comprises a light emitting layer, and
the light emitting layer comprises the compound for the organic optoelectronic device.

8. The organic optoelectronic device as claimed in claim 6, wherein
the organic layer comprises a light emitting layer,
a hole transport layer between the anode and the light emitting layer, and
a hole transport auxiliary layer between the light emitting layer and the hole transport layer,
wherein the hole transport auxiliary layer comprises the compound for the organic optoelectronic device.

9. A display device comprising the organic optoelectronic device as claimed in claim 6.
